SEQUENCE LISTING (34 sequences)

SEQ ID NO: 1 - protein, 121 aa, Artificial Sequence
  Synthetic Construct
EVQLVESGGGLVQPGGSLRLSCAASGFTFSDYYMTWVRQAPGKGLEWVAFIRNRARGYTSDHNPSVKGRFTISRDNAKNSLYLQMNSLRAEDTAVYYCARDRPSYYVLDYWGQGTTVTVSS

SEQ ID NO: 2 - protein, 5 aa, Artificial Sequence
  Synthetic Construct
DYYMT

SEQ ID NO: 3 - protein, 7 aa, Artificial Sequence
  Synthetic Construct
GFTFSDY

SEQ ID NO: 4 - protein, 10 aa, Artificial Sequence
  Synthetic Construct
GFTFSDYYMT

SEQ ID NO: 5 - protein, 8 aa, Artificial Sequence
  Synthetic Construct
RNRARGYT

SEQ ID NO: 6 - protein, 19 aa, Artificial Sequence
  Synthetic Construct
FIRNRARGYTSDHNPSVKG

SEQ ID NO: 7 - protein, 10 aa, Artificial Sequence
  Synthetic Construct
DRPSYYVLDY

SEQ ID NO: 8 - protein, 447 aa, Artificial Sequence
  Synthetic Construct
EVQLVESGGGLVQPGGSLRLSCAASGFTFSDYYMTWVRQAPGKGLEWVAFIRNRARGYTSDHNPSVKGRFTISRDNAKNSLYLQMNSLRAEDTAVYYCARDRPSYYVLDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCRVRCPRCPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSRLTVDKSRWQQGNVFSCSVMHEALHNHYTQKSLSLSPGK

SEQ ID NO: 9 - protein, 112 aa, Artificial Sequence
  Synthetic Construct
DIVMTQSPDSLAVSLGERATINCKSSQSLFNVRSRKNYLAWYQQKPGQPPKLLISWASTRESGVPDRFSGSGSGTDFTLTISSLQAEDVAVYYCKQSYDLFTFGSGTKLEIK

SEQ ID NO: 10 - protein, 17 aa, Artificial Sequence
  Synthetic Construct
KSSQSLFNVRSRKNYLA

SEQ ID NO: 11 - protein, 7 aa, Artificial Sequence
  Synthetic Construct
WASTRES

SEQ ID NO: 12 - protein, 8 aa, Artificial Sequence
  Synthetic Construct
KQSYDLFT

SEQ ID NO: 13 - protein, 219 aa, Artificial Sequence
  Synthetic Construct
DIVMTQSPDSLAVSLGERATINCKSSQSLFNVRSRKNYLAWYQQKPGQPPKLLISWASTRESGVPDRFSGSGSGTDFTLTISSLQAEDVAVYYCKQSYDLFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 115 aa, Artificial Sequence
  Synthetic Construct
EVQLLESGGGLVQPGGSLRLSCAASGFTFSSYPMSWVRQAPGKGLEWVSAIGGSGGSLPYADIVKGRFTISRDNSKNTLYLQMNSLRAEDTAVYYCARYWPMDIWGQGTLVTVSS

SEQ ID NO: 15 - protein, 7 aa, Artificial Sequence
  Synthetic Construct
GFTFSSY

SEQ ID NO: 16 - protein, 5 aa, Artificial Sequence
  Synthetic Construct
SYPMS

SEQ ID NO: 17 - protein, 10 aa, Artificial Sequence
  Synthetic Construct
GFTFSSYPMS

SEQ ID NO: 18 - protein, 6 aa, Artificial Sequence
  Synthetic Construct
GGSGGS

SEQ ID NO: 19 - protein, 17 aa, Artificial Sequence
  Synthetic Construct
AIGGSGGSLPYADIVKG

SEQ ID NO: 20 - protein, 6 aa, Artificial Sequence
  Synthetic Construct
YWPMDI

SEQ ID NO: 21 - protein, 441 aa, Artificial Sequence
  Synthetic Construct
EVQLLESGGGLVQPGGSLRLSCAASGFTFSSYPMSWVRQAPGKGLEWVSAIGGSGGSLPYADIVKGRFTISRDNSKNTLYLQMNSLRAEDTAVYYCARYWPMDIWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCEVECPECPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCEVKGFYPSDIAVEWESNGQPENNYKTTPPMLDSDGSFFLYSKLTVDKSRWQQGNVFSCSVMHEALHNHYTQKSLSLSPGK

SEQ ID NO: 22 - protein, 108 aa, Artificial Sequence
  Synthetic Construct
EIVLTQSPGTLSLSPGERATLSCRASQSVSSSYLAWYQQKPGQAPRLLMYDASIRATGIPDRFSGSGSGTDFTLTISRLEPEDFAVYYCQQYQSWPLTFGQGTKVEIK

SEQ ID NO: 23 - protein, 12 aa, Artificial Sequence
  Synthetic Construct
RASQSVSSSYLA

SEQ ID NO: 24 - protein, 7 aa, Artificial Sequence
  Synthetic Construct
DASIRAT

SEQ ID NO: 25 - protein, 9 aa, Artificial Sequence
  Synthetic Construct
QQYQSWPLT

SEQ ID NO: 26 - protein, 215 aa, Artificial Sequence
  Synthetic Construct
EIVLTQSPGTLSLSPGERATLSCRASQSVSSSYLAWYQQKPGQAPRLLMYDASIRATGIPDRFSGSGSGTDFTLTISRLEPEDFAVYYCQQYQSWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 117 aa, Artificial Sequence
  Synthetic Construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSS

SEQ ID NO: 28 - protein, 5 aa, Artificial Sequence
  Synthetic Construct
SYWIN

SEQ ID NO: 29 - protein, 17 aa, Artificial Sequence
  Synthetic Construct
NIYPGSSLTNYNEKFKN

SEQ ID NO: 30 - protein, 8 aa, Artificial Sequence
  Synthetic Construct
LSTGTFAY

SEQ ID NO: 31 - protein, 113 aa, Artificial Sequence
  Synthetic Construct
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIK

SEQ ID NO: 32 - protein, 17 aa, Artificial Sequence
  Synthetic Construct
KSSQSLWDSGNQKNFLT

SEQ ID NO: 33 - protein, 7 aa, Artificial Sequence
  Synthetic Construct
WTSYRES

SEQ ID NO: 34 - protein, 9 aa, Artificial Sequence
  Synthetic Construct
QNDYFYPHT

(19) United States
(12) Patent Application Publication
(10) Pub. No.: US 2023/0192878 A1
(43) Pub. Date: Jun. 22, 2023

(54) METHODS, THERAPIES AND USES FOR TREATING CANCER

(71) Applicant: PFIZER INC., NEW YORK, NY (US)

(72) Inventors: Nathalie Annie BARDY BOUXIN, Franconville La Garenne (FR); Eloisa Virginia BARRY, Canton, MA (US); John Andrew BLAKE-HASKINS, San Marcos, CA (US); Geoffrey Wing-Lynn CHAN, Cape May Point, NJ (US); Jeffrey CHOU, Fremont, CA (US); Mohamed A ELMELIEGY, San Diego, CA (US); Heike Iris KRUPKA, Hayward, CA (US); Kai Hsin LIAO, San Diego, CA (US); Erik Rene VANDENDRIES, Chestnut Hill, MA (US); Andrea VIQUEIRA, Madrid (ES); Paul Stephen WISSEL, St. Davids, PA (US); Anne YVER, Bois Colombes (FR)

(73) Assignee: PFIZER INC., NEW YORK, NY (US)

(21) Appl. No.: 17/924,975

(22) PCT Filed: May 13, 2021
(86) PCT No.: PCT/IB2021/054123

§ 371 Date: Nov. 11, 2022

Related U.S. Application Data
(60) Provisional application No. 63/024,016, filed on May 13, 2020.
(60) Provisional application No. 63/078,211, filed on Sep. 14, 2020.
(60) Provisional application No. 63/106,302, filed on Oct. 27, 2020.
(60) Provisional application No. 63/185,357, filed on May 6, 2021.

(51) Int. Cl. C07K 16/28; A61K 31/4168; A61P 35/00

(52) U.S. Cl. CPC C07K 16/2878; C07K 16/2818; A61K 31/4168; A61P 35/00

(57) ABSTRACT

The present disclosure describes single agent and combination therapies and uses for the treatment of cancer and/or cancer-associated diseases. The single agent and combinations therapies include a BCMA antibody.

### FIELD

The present invention relates to both single agent and combination therapies useful for the treatment of cancer and/or cancer-associated diseases. In particular, the invention relates to single agent and combination therapies which comprise a BCMA×CD3 bispecific antibody.

### BACKGROUND

B-cell maturation antigen (BCMA, CD269, or TNFRSF17) is a member of the tumor necrosis factor receptor (TNFR) superfamily. BCMA was identified in a malignant human T cell lymphoma containing a t(4; 16) translocation. The gene is selectively expressed in the B-cell lineage with the highest expression in plasma blasts and plasma cells, antibody secreting cells. BCMA binds two ligands, B-cell activation factor (BAFF) (also called B-lymphocyte stimulator (BLyS) and APOL-related leukocyte expressed ligand (TALL-1)) and a proliferation-inducing ligand (APRIL) with affinity of 1 μM and 16 nM, respectively. Binding of APRIL or BAFF to BCMA promotes a signaling cascade involving NF-kappa B, Elk-1, c-Jun N-terminal kinase and the p38 mitogen-activated protein kinase, which produce signals for cell survival and proliferation. BCMA is also expressed on malignant B cells and several cancers that involve B lymphocytes including multiple myeloma, plasmacytoma, Hodgkin's Lymphoma, and chronic lymphocytic leukemia. In autoimmune diseases where plasmablasts are involved such as systemic lupus erythematosus (SLE) and rheumatoid arthritis, BCMA expressing antibody-producing cells secrete autoantibodies that attack self. BCMA also found in a soluble form (i.e. soluble BCMA or sBCMA) in peripheral blood of multiple myeloma patients and can result in a sink for BCMA-specific therapies. Several BCMA-specific therapies are currently in development, however, multiple myeloma remains an incurable disease, and almost all patients have developed resistance to these agents and eventually relapse.

The programmed death 1 (PD-1) receptor and PD-1 ligands 1 and 2 (PD-L1 and PD-L2, respectively) play integral roles in immune regulation. Expressed on activated T cells, PD-1 is activated by PD-L1 (also known as B7-H1) and PD-L2 expressed by stromal cells, tumor cells, or both, initiating T-cell death and localized immune suppression (Dong et al., Nat Med 1999; 5:1365-69; Freeman et al. J Exp Med 2000; 192:1027-34), potentially providing an immune-tolerant environment for tumor development and growth. Conversely, inhibition of this interaction can enhance local T cell responses and mediate antitumor activity in nonclinical animal models (Iwai Y, et al. Proc Natl Acad Sci USA 2002; 99:12293-97). There are several antibodies that inhibit the interaction between PD-1 and one or both of its ligands PD-L1 and PD-L2 currently in development for treating cancer.

The Notch pathway is a conserved signaling pathway that contributes to cell fate determination, proliferation, angiogenesis, and apoptosis. A unique characteristic of the Notch pathway is that the ligands (Jagged-1, 2 and Delta-1, 3, 4) and receptors (Notch-1, 2, 3, 4) are both type I membrane proteins. After cell—cell direct contact, notch receptors are cleaved by γ-secretase, releasing an intracellular domain (NICD) that translocates into the nucleus to modulate transcription. γ-secretase inhibitors (GSIs) have been developed for a number of diseases, such as, Alzheimer's disease and cancer.

There remains a need for improved therapies for the treatment of cancers and/or cancer-associated diseases, such as multiple myeloma. Furthermore, there is a need for therapies having greater efficacy than existing therapies. Preferred combination therapies of the present invention show greater efficacy than treatment with either therapeutic agent alone.

### SUMMARY

This invention relates to therapies, including combination therapies for the treatment of cancer and/or cancer-associated diseases. Provided herein are methods of treating a cancer and/or a cancer-associated disease in a subject. Also provided are methods of inhibiting tumor growth or progression in a subject who has malignant cells. Also provided are methods of inhibiting metastasis of malignant cells in a subject. Also provided are methods of inducing tumor regression in a subject who has malignant cells.

Disclosed herein are methods of treating a cancer and/or a cancer-associated disease in a subject comprising administering to the subject a combination therapy which comprises a first therapeutic agent and a second therapeutic agent. The invention disclosed herein is further directed to a medicament comprising a first therapeutic agent and a second therapeutic agent for use in treating cancer and/or a cancer-associated disease in a subject. The invention is further directed to a first therapeutic agent for use in treating cancer and/or a cancer-associated disease in a subject, wherein the first therapeutic agent is administered in combination with a second therapeutic agent.

In some aspects, the first therapeutic agent is a B-cell maturation antigen (BCMA)-specific therapeutic. In some aspects, the second therapeutic agent is an anti-PD-1 antibody, an anti-PD-L1 antibody, an immunomodulating agent or a gamma secretase inhibitor (GSI).

In some aspects, the first therapeutic agent is a BCMA bispecific antibody. In some aspects, the second therapeutic agent is an anti-PD-1 antibody. In another aspect, the second therapeutic agent is an anti-PD-L1 antibody. In another aspect, the second therapeutic agent is an immunomodulating agent. In another aspect, the second therapeutic agent is a GSI.

In some aspects, the first therapeutic agent is a BCMA bispecific antibody and the second therapeutic agent is an anti-PD-1 antibody. In another aspect, the first therapeutic agent is a BCMA bispecific antibody and the second therapeutic agent is an anti-PD-L1 antibody. In another aspect, the first therapeutic agent is a BCMA bispecific antibody and the second therapeutic agent is an immunomodulating agent. In another aspect, the first therapeutic agent is a BCMA bispecific antibody and the second therapeutic agent is a GSI.

In some aspects, the combination therapy further comprises a third, fourth or fifth therapeutic agent. In some aspects, the combination therapy further comprises a chemotherapeutic agent. In some aspects, the therapeutic agents are administered to the subject simultaneously, separately, or sequentially.

In some aspects, the BCMA bispecific antibody is PF-06863135, the anti-PD-1 antibody is sasanlimab, the immunomodulating agent is lenalidomide or pomalidomide, and/or the GSI is nirogacestat or a pharmaceutically acceptable salt thereof. In one aspect, the BCMA bispecific antibody is PF-06863135. In one aspect, the anti-PD-1 antibody is sasanlimab. In one aspect, the immunomodulating agent is lenalidomide. In another aspect, the immunomodulating agent is pomalidomide. In one aspect, the GSI is nirogacestat or a pharmaceutically acceptable salt thereof.

In some aspects, at least one of the therapeutic agents is administered to a subject in an intravenous (IV), subcutaneous (SC) or oral dose.

In some aspects, at least one of the therapeutic agents is administered to a subject at a dose of about 0.01 μg/kg, 0.02 μg/kg, 0.03 μg/kg, 0.04 μg/kg, 0.05 μg/kg, 0.06 μg/kg, 0.07 μg/kg, 0.08 μg/kg, 0.09 μg/kg, 0.1 μg/kg, 0.2 μg/kg, 0.3 μg/kg, 0.4 μg/kg, 0.5 μg/kg, 0.6 μg/kg, 0.7 μg/kg, 0.8 μg/kg, 0.9 μg/kg, 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 15 μg/kg, 20 μg/kg, 25 μg/kg, 30 μg/kg, 35 μg/kg, 40 μg/kg, 45 μg/kg, 50 μg/kg, 60 μg/kg, 70 μg/kg, 80 μg/kg, 90 μg/kg, 100 μg/kg, 110 μg/kg, 120 μg/kg, 130 μg/kg, 140 μg/kg, 150 μg/kg, 200 μg/kg, 250 μg/kg, 300 μg/kg, 400 μg/kg, 500 μg/kg, 600 μg/kg, 700 μg/kg, 800 μg/kg, 900 μg/kg, 1000 μg/kg, 1200 μg/kg or 1400 μg/kg or higher.

In some aspects, at least one of the therapeutic agents is administered to a subject at a dose from about 1 mg/kg to about 1000 mg/kg, from about 2 mg/kg to about 900 mg/kg, from about 3 mg/kg to about 800 mg/kg, from about 4 mg/kg to about 700 mg/kg, from about 5 mg/kg to about 600 mg/kg, from about 6 mg/kg to about 550 mg/kg, from about 7 mg/kg to about 500 mg/kg, from about 8 mg/kg to about 450 mg/kg, from about 9 mg/kg to about 400 mg/kg, from about 5 mg/kg to about 200 mg/kg, from about 2 mg/kg to about 150 mg/kg, from about 5 mg/kg to about 100 mg/kg, from about 10 mg/kg to about 100 mg/kg, or from about 10 mg/kg to about 60 mg/kg; or

In some aspects, at least one of the therapeutic agents is administered to a subject at a fixed dose of about 0.05 μg, 0.2 μg, 0.5 μg, 1 μg, 10 μg, 100 μg, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 15 mg, 20 mg, 25 mg, 30 mg, 40 mg, 50 mg, 60 mg, 70 mg, 75 mg, 80 mg, 90 mg, 100 mg, 125 mg, 150 mg, 175 mg, 200 mg, 225 mg, 250 mg, 275 mg, 300 mg, 350 mg, 400 mg, 450 mg, 500 mg, 550 mg, 600 mg, 350 mg, 700 mg, 750 mg, 800 mg, 900 mg, 1000 mg or 1500 mg or higher.

In some aspects, at least one of the therapeutic agents is administered to a subject at least once daily, once a day, twice a day, three times a day, four times a day, once every two days, once every three days, once a week, once every two weeks, once every three weeks, once every four weeks, once every 30 days, once every five weeks, once every six weeks, once a month, once every two months, once every three months, or once every four months.

In some aspects, the cancer and/or cancer-associated disease is a B-cell related cancer and/or cancer-associated disease. In some aspects, the B-cell related cancer and/or cancer-associated disease is selected from multiple myeloma, malignant plasma cell neoplasm, lymphoma, Hodgkin's lymphoma, nodular lymphocyte predominant Hodgkin's lymphoma, Kahler's disease and Myelomatosis, plasma cell leukemia, bony and extramedullary plasmacytoma with multiple myeloma, solid bony and extramedullary plasmacytoma, monoclonal gammopathy of unknown significance (MGUS), smoldering myeloma, light chain amyloidosis, osteosclerotic myeloma, B-cell prolymphocytic leukemia, hairy cell leukemia, B-cell non-Hodgkin's lymphoma (NHL), acute myeloid leukemia (AML), chronic lymphocytic leukemia (CLL), acute lymphocytic leukemia (ALL), chronic myeloid leukemia (CML), follicular lymphoma, Burkitt's lymphoma, marginal zone lymphoma, mantle cell lymphoma, large cell lymphoma, precursor B-lymphoblastic lymphoma, myeloid leukemia, Waldenstrom's macroglobulienemia, diffuse large B cell lymphoma, mucosa-associated lymphatic tissue lymphoma, small cell lymphocytic lymphoma, primary mediastinal (thymic) large B-cell lymphoma, lymphoplasmactyic lymphoma, marginal zone B cell lymphoma, splenic marginal zone lymphoma, intravascular large B-cell lymphoma, primary effusion lymphoma, lymphomatoid granulomatosis, T cell/histiocyte-rich large B-cell lymphoma, primary central nervous system lymphoma, primary cutaneous diffuse large B-cell lymphoma (leg type), EBV positive diffuse large B-cell lymphoma of the elderly, diffuse large B-cell lymphoma associated with inflammation, ALK-positive large B-cell lymphoma, plasmablastic lymphoma, large B-cell lymphoma arising in HHV8-associated multicentric Castleman disease, B-cell lymphoma unclassified with features intermediate between diffuse large B-cell lymphoma and Burkitt lymphoma, B-cell lymphoma unclassified with features intermediate between diffuse large B-cell lymphoma and classical Hodgkin lymphoma, and other B-cell related lymphoma. In some aspects, the B-cell related cancer is multiple myeloma. In some aspects, the multiple myeloma is relapsed/refractory multiple myeloma.

Also provided herein are methods of treating multiple myeloma in a subject comprising administering to the subject a combination therapy which comprises a first therapeutic agent and a second therapeutic agent, wherein the first therapeutic agent is a B-cell maturation antigen (BCMA) bispecific antibody and the second therapeutic agent is an anti-PD-1 antibody, an anti-PD-L1 antibody, an immunomodulating agent or a gamma secretase inhibitor (GSI). Also provided herein is a first therapeutic agent for use in methods of treating multiple myeloma in a subject, wherein the first therapeutic agent is a B-cell maturation antigen (BCMA) bispecific antibody, and is administered in combination with a second therapeutic agent selected from an anti-PD-1 antibody, an anti-PD-L1 antibody, an immunomodulating agent or a gamma secretase inhibitor (GSI). In some aspects, the first therapeutic agent is a BCMA bispecific antibody and the second therapeutic agent is an anti-PD-1 antibody. In another aspect, the first therapeutic agent is a BCMA bispecific antibody and the second therapeutic agent is an anti-PD-L1 antibody. In another aspect, the first therapeutic agent is a BCMA bispecific antibody and the second therapeutic agent is an immunomodulating agent. In another aspect, the first therapeutic agent is a BCMA bispecific antibody and the second therapeutic agent is a GSI.

Also provided are methods of treating multiple myeloma in a subject comprising administering to the subject a combination therapy which comprises a first therapeutic agent and a second therapeutic agent, wherein the first therapeutic agent is PF-06863135 and the second therapeutic agent is sasanlimab.

Also provided are methods of treating multiple myeloma in a subject comprising administering to the subject a combination therapy which comprises a first therapeutic agent and a second therapeutic agent, wherein the first therapeutic agent is PF-06863135 and the second therapeutic agent is lenalidomide.

Also provided are methods of treating multiple myeloma in a subject comprising administering to the subject a combination therapy which comprises a first therapeutic agent and a second therapeutic agent, wherein the first therapeutic agent is PF-06863135 and the second therapeutic agent is pomalidomide.

Also provided are methods of treating multiple myeloma in a subject comprising administering to the subject a combination therapy which comprises a first therapeutic agent and a second therapeutic agent, wherein the first therapeutic agent is PF-06863135 and the second therapeutic agent is nirogacestat.

Also provided are methods of treating cancer in a subject comprising administering to the subject PF-06863135 according to a dosing regimen.

In some embodiments, the dosing regimen is:
(a) 0.1, 0.3, 1, 3, 10, 30, 50 or 100 μg/kg once per week (Q1W) intravenously (IV).
(b) 0.1, 0.3, 1, 3, 10, 30, 50 or 100 μg/kg once every two weeks (Q2W) IV;
(c) about 0.5 to 10 mg Q1W IV or Q2W IV;
(d) about 0.5, 1, 2, 3, 4, 5, 6, 7, 7.5 or 8 mg Q1W IV or Q2V IV.
(e) a priming dosing of a single priming dose of about 0.5, 1, 2, 3, 4, 5, 6, 7.5 or 8 mg Q1W IV for one week followed by a first treatment dosing of about 6, 7, 7.5, 8, 9 or 10 mg Q1W IV or Q2W IV, wherein priming dose is less than a single dose in the treatment dosing; or
(f) a priming dosing of a single priming dose of about 0.5, 1, 2, 3, 4, 5, 6, 7, 7.5 or 8 mg Q1W for one week followed by a first treatment dosing of about 6, 7, 7.5, 8, 9 or 10 mg Q1W IV for 2 to 20, 21, 22, 23, 24, 25 to 46, 47 or 48 weeks, followed by a second treatment dosing of about 6, 7, 7.5, 8, 9 or 10 Q2W IV, wherein the priming dose is less than a single dose in the first treatment dosing.

In another aspect of the invention, the dosing regimen is:
(a) 80, 130, 215, 360, 600 or 1000 μg/kg Q1W subcutaneously (SC);
(b) 80, 130, 215, 360, 600 or 1000 μg/kg Q2W SC;
(c) about 16 to 80 mg Q1W SC or Q2W SC;
(d) about 16 to 20, 40 to 44, or 76 to 80 mg Q1W SC;
(e) about 16 to 20, 40 to 44, or 76 to 80 mg Q2W SC;
(f) about 40 mg Q1W SC or Q2W SC;
(g) about 44 mg Q1W SC or Q2W SC;
(h) about 76 mg Q1W SC or Q2W SC;
(i) about 80 mg Q1W SC or Q2W SC;
(j) a priming dosing of about 44 mg Q1W SC for 1-4 weeks, or a priming dosing of about 32 mg Q1W SC for 1-4 weeks, followed by a first treatment dosing of about 76 mg Q1W SC or Q2W SC;
(k) a priming dosing of about 40 mg Q1W SC for 1-4 weeks, followed by a first treatment dosing of about 80 mg Q1W SC or Q2W SC;
(l) a priming dosing of about 44 mg Q1W SC for 1-4 weeks, or a priming dosing of about 32 mg Q1W SC for 1-4 weeks, followed by a first treatment dosing of about 76 mg Q1W SC for 2 to 20, 21, 22, 23, 24, 25 to 46, 47 or 48 weeks, followed by a second treatment dosing of about 76 mg Q2W SC;
(m) a priming dosing of about 40 mg Q1W SC for 1-4 weeks, followed by a first treatment dosing of about 80 mg Q1W SC for 2 to 20, 21, 22, 23, 24, 25 to 46, 47 or 48 weeks, followed by a second treatment dosing of about 80 mg Q2W SC;
(n) a priming dosing of about 44 mg Q1W SC for 1 week, followed by a first treatment dosing of about 76 mg Q1W SC or Q2W SC;
(o) a priming dosing of about 32 mg Q1W SC for 1 week, followed by a first treatment dosing of about 76 mg Q1W SC or Q2W SC;
(p) a priming dosing of about 40 mg Q1W SC for 1 week, followed by a first treatment dosing of about 80 mg Q1W SC or Q2W SC.
(q) a priming dosing of about 44 mg Q1W SC for 1 week, followed by a first treatment dosing of about 76 mg Q1W SC for 2 to 20, 21, 22, 23, 24, 25 to 46, 47 or 48 weeks, followed by a second treatment dosing of about 76 mg Q2W SC;
(r) a priming dosing of about 44 mg Q1W SC for 1 week, followed by a first treatment dosing of about 76 mg Q1W SC for 23 weeks, followed by a second treatment dosing of about 76 mg Q2W SC;
(s) a priming dosing of about 44 mg Q1W SC for 1 week, followed by a first treatment dosing of about 76 mg Q1W SC for 24 weeks, followed by a second treatment dosing of about 76 mg Q2W
(t) a priming dosing of about 32 mg Q1W SC for 1 week, followed by a first treatment dosing of about 76 mg Q1W SC for 2 to 20, 21, 22, 23, 24, 25 to 46, 47 or 48 weeks, followed by a second treatment dosing of about 76 mg Q2W SC;
(u) a priming dosing of about 40 mg Q1W SC for 1 week, followed by a first treatment dosing of about 80 mg Q1W SC for 2 to 20, 21, 22, 23, 24, 25 to 46, 47 or 48 weeks, followed by a second treatment dosing of about 80 mg Q2W SC; or
(v) a priming dosing of about 40 mg Q1W SC for 1 week, followed by a first treatment dosing of about 80 mg Q1W for 23 or 24 weeks, followed by a second treatment dosing of about 80 mg Q2W SC.

In some embodiments, the priming dosing is administered for only one week in a single priming dose of 44 mg Q1W SC, 40 mg Q1W SC or 32 Q1W SC.

Also provided are methods of treating cancer in a subject, comprising administering to the subject PF06863135, (a) a single priming dose of about 32 mg SC or about 44 mg SC in week 1, or both a first priming dose of about 12 mg SC and a second priming dose of about 32 mg SC in week 1, and (b) a first treatment dosing of about 76 mg Q1W SC starting on week 2, wherein week 1, week 2 and any subsequent weeks refer to the first, second and any subsequent weeks when the subject is administered PF06863135, and PF6863135 is administered to the subject as a pharmaceutical product comprising PF06863135.

In some embodiments, the subject is administered PF06863135 of a single priming dose of about 44 mg SC in week 1. In some embodiments, the subject is administered a first priming dose of about 12 mg SC on day 1 of week 1, and a second priming does of about 32 mg SC on day 4 of week 1.

In some embodiments, the method further comprising administering to the subject PF06863135 in a second treatment dosing of about 76 mg Q2W SC starting on week 25 or the first week of cycle 7, wherein PF06863135 in the first treatment dosing is administered until the end of week 24, or the end of cycle 6, wherein a cycle is 28 days, and cycle 1, cycle 2 and subsequent cycles refer to the first, second and subsequent cycles when the subject is administered PF06863135.

In some embodiments, the subject is administered PF06863135 in the first treatment dosing of about 76 mg Q1W SC, and after receiving at least 23 weeks of such first treatment dosing, the subject is administered PF06863135 in a second treatment dosing of 76 mg Q2W or continue to be administered PF06863135 in the first treatment dosing. In some embodiments, the subject is administered PF06863135 in the second treatment dosing after receiving at least 23 weeks of the first treatment dosing, according to a respective regulatory label of the pharmaceutical product, or according to the subject's response. In some embodiments, the subject continues to be administered PF06863135 in the first treatment dosing after receiving at least 23 weeks of the first treatment dosing, unless the subject has demonstrated an IMWG response of a partial response or better, with response persisting for at least one month, at least two months, at least three months, at least one cycle, at least two cycles or at least three cycles, after received at least six cycles of treatment, and each cycle is 28 days and the first cycle starts on the day when the subject is administered the single priming dose or the first priming dose of PF06863135.

Also provided are methods of treating cancer in a subject comprising administering to the subject PF-06863135 according to a dosing regimen of:
(a) a priming dosing of about 32 mg Q1W SC for 1 week, followed by a first treatment dosing of about 44 mg Q1W SC;
(b) a priming dosing of about 32 mg Q1W SC for 1 week, followed by a first treatment dosing of about 44 mg Q2W SC;
(c) a priming dosing of about 32 mg Q1W SC for 1 week, followed by a first treatment dosing of about 44 mg Q1W SC for 2 to 20, 21, 22, 23, 24, 25 to 46, 47 or 48 weeks, and a second treatment dosing of about 44 mg Q2W SC; or
(d) a priming dosing of about 32 mg Q1W SC for 1 week, followed by a first treatment dosing of about 44 mg Q1W SC for 23 or 24 weeks, and a second treatment dosing of about 44 mg Q2W SC.

In some embodiments, PF-06863135 is administered to the subject in a priming dosing of about 32 mg Q1W SC for 1 week, followed by a first treatment dosing of about 44 mg Q1W SC. In some embodiments, PF-06863135 is administered to the subject in a priming dosing of about 32 mg Q1W SC for 1 week, followed by a first treatment dosing of about 44 mg Q1W SC for 23 or 24 weeks followed by a second treatment dosing of about 44 mg Q2W SC.

Also provided are methods of treating cancer in a subject comprising administering PF-06863135 to the subject, subcutaneously, a first treatment dosing for 23, 24 or 25 weeks, followed by a second treatment dosing.

In some embodiments, the first treatment dosing is about 4 mg Q1W, and the second treatment dosing is about 4 mg Q1W or about 4 mg Q2W. In some embodiments, the first treatment dosing is about 12 mg Q1W, and the second treatment dosing is about 12 mg Q1W or about 12 mg Q2W. In some embodiments, the first treatment dosing is about 24 mg Q1W, and the second treatment dosing is about 24 mg Q1W or about 24 mg Q2W. In some embodiments, the first treatment dosing is about 32 mg Q1W, and the second treatment dosing is about 32 mg Q1W or about 32 mg Q2W. In some embodiments, the first treatment dosing is about 44 mg Q1W, and the second treatment dosing is about 44 mg Q1W or about 44 mg Q2W. In some embodiments, the first treatment dosing is about 76 mg Q1W, and the second treatment dosing is about 76 mg Q1W or about 76 mg Q2W. In some embodiments, the first treatment dosing is about 4 mg Q1W, and the second treatment dosing is about 4 mg Q2W. In some embodiments, the first treatment dosing is about 12 mg Q1W, and the second treatment dosing is about 12 mg Q2W. In some embodiments, the first treatment dosing is about 24 mg Q1W, and the second treatment dosing is about 24 mg Q2W. In some embodiments, the first treatment dosing is about 32 mg Q1W, and the second treatment dosing is about 32 mg Q2W. In some embodiments, the first treatment dosing is about 44 mg Q1W, and the second treatment dosing is about 44 mg Q2W. In some embodiments, the first treatment dosing is about 76 mg Q1W, and the second treatment dosing is about 76 mg Q2W.

In some embodiments, if the dose amount of the first treatment dosing is 32 mg or higher, the method further comprising administering to the subject PF06863135 in a priming dosing, and the priming dosing is administered for one week, the first dose in the first treatment dosing is administered in the week immediately after the week when the priming dosing is administered. In some embodiments, the priming dosing is a single priming dose, and the single priming dose is about 24 mg. In some embodiments, the priming dosing comprises a first priming dose of about 4 mg and a second priming dose of about 20 mg, and the two priming doses are administered on two different days and the first priming dose is administered before the second priming dose is administered. In some embodiments, the priming dosing comprises a first priming dose of about 8 mg and a second priming dose of about 16 mg, and the two priming doses are administered on two different days and the first priming dose is administered before the second priming dose is administered. In some embodiments, the priming dosing comprises a first priming dose of about 12 mg and a second priming dose of about 12 mg, and the two priming doses are administered on two different days and the first priming dose is administered before the second priming dose is administered. In some embodiments, the priming dosing comprises a first priming dose of about 8 mg and a second priming dose of about 24 mg, and the two priming doses are administered on two different days and the first priming dose is administered before the second priming dose is administered. In some embodiments, the priming dosing comprises a first priming dose of about 4 mg and a second priming dose of about 28 mg, and the two priming doses are administered on two different days and the first priming dose is administered before the second priming dose is administered.

In some embodiments, the subject is administered PF06863135 of the second treatment dosing for 6 to 18 cycles, wherein a cycle is 21 days or 28 days, thereafter, the subject is administered a third treatment dosing of PF06863135 subcutaneously. In some embodiments, the third treatment dosing is about 4 mg Q2W or about 4 mg Q4W. In some embodiments, the third treatment dosing is about 12 mg Q2W or about 12 mg Q4W. In some embodiments, the third treatment dosing is about 24 mg Q2W or about 24 mg Q4W. In some embodiments, the third treatment dosing is about 32 mg Q2W or about 32 mg Q4W. In some embodiments, the third treatment dosing is about 44 mg Q2W, about 44 mg Q4W. In some embodiments, the third treatment dosing is about 76 mg Q2W or about 76 mg Q4W.

In some embodiments, the first treatment dosing is about 4 mg Q1W, the second treatment dosing is about 4 mg Q2W and the third treatment dosing is about 4 mg Q4W. In some embodiments, the first treatment dosing is about 12 mg Q1W, the second treatment dosing is about 12 mg Q2W and the third treatment dosing is about 12 mg Q4W. In some embodiments, the first treatment dosing is about 24 mg Q1W, the second treatment dosing is about 24 mg Q2W and the third treatment dosing is about 32 mg Q4W. In some embodiments, the first treatment dosing is about 32 mg Q1W, the second treatment dosing is about 32 mg Q2W and the third treatment dosing is about 24 mg Q4W. In some embodiments, the first treatment dosing is about 44 mg Q1W, the second treatment dosing is about 44 mg Q2W and the third treatment dosing is about 44 mg Q4W. In some embodiments, the first treatment dosing is about 76 mg Q1W, the second treatment dosing is about 76 mg Q2W and the third treatment dosing is about 76 mg Q4W.

Also provided are methods of treating cancer in a subject comprising administering PF-06863135 to a subject
(a) a first treatment dosing of about 32 mg to about 76 mg Q1W SC, starting in week 1; or
(b) a priming dosing during week 1, and a first treatment dosing starting in week 2, wherein the priming dosing is (i) a first priming dose of about 4 mg SC to about 32 mg SC, and a second priming dose of about 12 mg SC to about 44 mg SC, wherein the first priming dose and the second priming dose are administered sequentially in week 1, or (ii) a single priming dose of about 24 mg to about 44 mg SC, and wherein the first treatment dosing is about 32 mg to about 76 mg Q1W SC or about 32 mg to about 152 mg Q2W SC, starting in week 2, and wherein the dose amount of the first treatment dosing is higher than the dose amount of each of the respective single priming dose, first priming dose and second priming dose;

wherein week 1, week 2 and any subsequent weeks refer to the first, second and any subsequent weeks when the subject is administered PF06863135 respectively, and PF06863135 is administered to the subject as a pharmaceutical product comprising PF06863135.

In some embodiments, the subject is administered a priming dosing of a single priming dose of about 24 mg SC, about 32 mg SC or about 44 mg SC in week 1. In some embodiments, the subject is administered a priming dosing of a first priming dose of about 12 mg SC and a second priming dose of about 32 mg SC in week 1. In some embodiments, the subject is administered a priming dosing of a single priming dose of about 4 mg, about 8 mg, about 12 mg, or about 24 mg during week 1. In some embodiments, the subject is administered a priming dosing of a first priming dose and a second priming dose. In some embodiments, the first priming dose is about 4 mg, and the second priming dose is about 20 mg. In some embodiments, the first priming dose is about 8 mg and the second priming dose is about 16 mg. In some embodiments, the first priming dose is about 12 mg and the second priming dose is about 12 mg. In some embodiments, the first priming dose is about 8 mg and the second priming dose is about 24 mg.

In some embodiments, the first treatment dosing is about 32 mg Q1W SC or about 32 mg Q2W SC. In some embodiments, the first treatment dosing is about 44 mg Q1W SC, or about 44 mg Q2W SC. In some embodiments, the subject is administered the first treatment dosing until at least the end of cycle 1 or until at least the end of cycle 6, wherein a cycle is 21 days or 28 days, cycle 1 starts on day 1 of week 1, day 1 of week 2, or day 1 of week 3, and cycle 1, cycle 2 and subsequent cycles refer to the first, second and subsequent cycles when the subject is administered PF06863135 respectively.

In some embodiments, the method further comprising administering to the subject PF06863135 in a second treatment dosing of about 32 mg to about 152 mg Q2W SC, about 32 mg to about 152 mg Q3W SC, or about 32 mg to about 152 mg Q4W SC, after the subject is no longer on the first treatment dosing, wherein the second treatment dosing is at a dose frequency that is less frequent than the respective first treatment dosing, or the second treatment dosing has a lower dose amount than the first treatment dosing. In some embodiments, wherein after the first treatment dosing is administered to the subject until at least the end of cycle 6, the second treatment dosing of PF06863135 is administered to the subject instead of the first treatment dosing, or the subject may continue to be administered the first treatment dosing, and wherein the second treatment dosing is about 32 mg to about 152 mg Q2W SC, about 32 mg to about 152 mg Q3W SC, or about 32 mg to about 152 mg Q4W SC, wherein the second treatment dosing is at a dose frequency that is less frequent than the first treatment dosing, or the second treatment dosing has a lower dose amount than the first treatment dosing. In some embodiments, wherein (i) the first treatment dosing is about 32 mg Q1W SC and the second treatment dosing is about 32 mg Q2W SC, 32 mg Q3W SC, 32 mg Q4W SC, 44 mg Q2W SC, 44 mg Q3W SC, 44 mg Q4W SC, 76 mg Q3W SC, 76 mg Q4W SC, 116 mg Q4W SC or 152 mg Q4W SC, or (ii) the first treatment dosing is about 32 Q2W SC and the second treatment dosing is about 32 mg Q3W SC, 32 mg Q4W SC, 44 mg Q3W SC, 44 mg Q4W SC, 76 mg Q3W SC, 76 mg Q4W SC, 116 mg Q4W SC or 152 mg Q4W SC. In some embodiments, wherein (i) the first treatment dosing is about 44 mg Q1W SC and the second treatment dosing is about 44 mg Q2W SC, 44 mg Q3W SC, 44 mg Q4W SC, 76 mg Q2W SC, 76 mg Q3W SC, 76 mg Q4W SC, 116 mg Q4W SC or about 152 mg Q4W SC, or (ii) the first treatment dosing is about 44 mg Q2W SC, and the second treatment dosing is about 32 mg Q2W SC, 44 mg Q3W SC, 76 mg Q3W SC, 116 mg Q3W SC, 152 mg Q3W SC, 32 mg Q4W SC, 44 mg Q4W SC, 76 mg Q4W SC, 116 mg Q4W SC, or about 152 mg Q4W SC. In some embodiments, the second treatment dosing is administered to the subject according to a respective regulatory label of the pharmaceutical product. In some embodiments, the second treatment dosing is administered to the subject according to the subject's response to the first treatment dosing. In some embodiments, the subject continues to be administered the first treatment dosing unless the subject has demonstrated an IMWG response of a partial response or better, with response persisting for at least one month, at least two months, at least three months, at least one cycle, at least two cycles or at least three cycles while the subject is on the first treatment dosing.

In some embodiments, the first treatment dosing is (i) about 76 mg Q1W SC, (ii) about 76 mg Q2W SC, or (iii) about 76 mg Q1W SC for three weeks followed by about 116 mg Q1W SC or (iv) about 76 mg Q1W SC for three weeks followed by about 152 mg Q1W SC. In some embodiments, the subject is administered the first treatment dosing until at least the end of cycle 1, at least the end of cycle 3, or at least the end of cycle 6, wherein a cycle is 21 days or 28 days, and cycle 1 starts on day 1 of week 1, day 1 of week 2, or day 1 of week 3, and cycle 1, cycle 2 and subsequent cycles refer to the first, second and subsequent cycles when the subject is administered PF06863135 respectively. In some embodiments, the method further comprising administering to the subject PF06863135 in a second treatment dosing of about 44 mg to about 152 mg Q2W SC, about 44 mg to about 152 mg Q3W SC, or about 44 mg to about 152 mg Q4W SC, after the subject is no longer on the first treatment dosing, wherein the second treatment dosing is at a dose frequency that is less frequent than the first treatment dosing, or the second treatment dosing has a lower dose amount than the first treatment dosing. In some embodiments, wherein after the first treatment dosing is administered to the subject until at least the end of cycle 6, a second treatment dosing of about 44 mg to about 152 mg Q2W SC, about 44 mg to about 152 mg Q3W SC, or about 44 mg to about 152 mg Q4W SC is be administered to the subject instead of the first treatment dosing, or the subject may continue to be administered the first treatment dosing, wherein the second treatment dosing is at a dose frequency that is less frequent than the respective first treatment dosing, or the second treatment dosing has a lower dose amount than that of the first treatment dosing. In some embodiments, the first treatment dosing is about 76 mg Q1W SC and the second treatment dosing of about 44 mg Q2W SC, about 76 mg Q2W SC, about 116 mg Q2W SC, about 152 mg Q2W SC, about 44 mg Q3W SC, about 76 mg Q3W SC, about 116 mg Q3W SC, about 152 mg Q3W SC, about 44 mg Q4W SC, about 76 mg Q4W SC, about 116 mg Q4W SC or about 152 mg Q4W SC. In some embodiments, the first treatment dosing is about 76 mg Q2W SC, and the second treatment dosing is about 44 mg Q2W SC, about 44 mg Q3W SC, about 76 mg Q3W SC, about 116 mg Q3W SC, about 152 mg Q3W SC, about 44 mg Q4W SC, about 76 mg Q4W SC, about 116 mg Q4W SC or about 152 mg Q4W SC. In some embodiments, the first treatment dosing is about 76 mg Q1W, the second treatment dosing is about 76 mg Q2W. In some embodiments, the first treatment dosing is about 76 mg Q2W, the second treatment dosing is about 76 mg Q4W. In some embodiments, the second treatment dosing is administered to the subject according to a respective regulatory label of the pharmaceutical product. In some embodiments, the second treatment dosing is administered to the subject according to the subject's response to the first treatment dosing. In some embodiments, the second treatment dosing is administered to the subject if the subject has demonstrated an IMWG response of a partial response or better, with response persisting for at least one month, at least two months, at least three months, at least one cycle, at least two cycles or at least three cycles while the subject is on the first treatment dosing. In some embodiments, the subject is administered PF06863135 in the first treatment dosing until the end of cycle 1, followed by the second treatment dosing, wherein a cycle is 21 days or 28 days, cycle 1 starts on day 1 of week 1, or day 1 of week 2, or day 1 of week 3, and cycle 1, cycle 2 and subsequent cycles refer to the first, second and subsequent cycles when the subject is administered PF06863135 respectively. In some embodiments, the second treatment dosing is administered until at least the end of cycle 6, and thereafter a third treatment dosing of about 76 mg to about 152 mg Q3W SC or about 76 mg to about 152 mg Q4W SC is administered to the subject instead of the second treatment dosing, or the subject continues to be administered the second treatment dosing. In some embodiments, the second treatment dosing is administered until at least the end of cycle 6, and thereafter a third treatment dosing of about 76 mg to about 152 mg Q3W SC or about 76 mg to about 152 mg Q4W SC is administered. In some embodiments, the second treatment dosing is administered until the end of cycle 6, the first dose in the third treatment dosing starts in cycle 7 and the third treatment dosing is 116 mg Q4W SC or 152 mg Q4W SC. In some embodiments, the subject is administered PF06863135 in the third treatment dosing after receiving the second treatment dosing until at least cycle 6, according to a respective regulatory label of the pharmaceutical product, or according to the subject's response. In some embodiments, the subject continues to be administered PF06863135 in the second treatment dosing until at least cycle 6, unless the subject has demonstrated an IMWG response of a partial response or better, with response persisting for at least one month, at least two months, at least three months, at least one cycle, at least two cycles or at least three cycles while the subject is on the second treatment dosing. In some embodiments, the first treatment dosing is about 76 mg Q1W SC, the second treatment dosing is about 116 mg Q2W SC and the third treatment dosing is about 116 mg Q4W SC. In some embodiments, the first treatment dosing is about 76 mg Q1W SC, the second treatment dosing is about 152 mg Q2W SC and the third treatment dosing is about 152 mg Q4W SC.

In some embodiments, the method comprising administering to the subject a first treatment dosing of about 32 mg Q1W for 23, 24 or 25 weeks, followed by a second treatment dosing of about 32 mg Q1W or about 32 mg Q2W for 6 to 18 cycles, followed by a third treatment dosing of about 32 mg Q2W or about 32 mg Q4W, wherein a cycle is 21 or 28 days. In some embodiments, the second treatment dosing is about 32 mg Q2W and the third treatment dosing is about 32 mg Q4W.

In some embodiments, the method comprising administering to the subject a first treatment dosing of about 44 mg Q1W for 23, 24 or 25 weeks, followed by a second treatment dosing of about 44 mg Q1W or about 44 mg Q2W for 6 to 18 cycles, followed by a third treatment dosing of about 44 mg Q2W or about 44 mg Q4W, wherein a cycle is 21 or 28 days. In some embodiments, the second treatment dosing is about 44 mg Q2W and the third treatment dosing is about 44 mg Q4W.

In some embodiments, the method comprising administering to the subject a first treatment dosing of about 76 mg Q1W for 23, 24 or 25 weeks, followed by a second treatment dosing of about 76 mg Q1W or about 76 mg Q2W for 6 to 18 cycles, followed by a third treatment dosing of about 76 mg Q2W or about 76 mg Q4W, wherein a cycle is 21 or 28 days. In some embodiments, the second treatment dosing is about 76 mg Q2W and the third treatment dosing is about 76 mg Q4W.

In some embodiments, the method comprising administering to the subject a first treatment dosing of about 116 mg Q1W for 23, 24 or 25 weeks, followed by a second treatment dosing of about 116 mg Q1W or about 116 mg Q2W for 6 to 18 cycles, followed by a third treatment dosing of about 116 mg Q2W or about 116 mg Q4W, wherein a cycle is 21 or 28 days. In some embodiments, the second treatment dosing is about 116 mg Q2W and the third treatment dosing is about 116 mg Q4W.

In some embodiments, the method comprising administering to the subject a first treatment dosing of about 152 mg Q1W for 23, 24 or 25 weeks, followed by a second treatment dosing of about 152 mg Q1W or about 152 mg Q2W for 6 to 18 cycles, followed by a third treatment dosing of about 152 mg Q2W or about 152 mg Q4W, wherein a cycle is 21 or 28 days. In some embodiments, the second treatment dosing is about 152 mg Q2W and the third treatment dosing is about 152 mg Q4W.

In some embodiments, a cycle is 21 days when the subject is on Q1W or Q3W dosing frequency of PF06863135 and a cycle is 28 days when the subject is on a Q2W or Q4W dosing frequency of PF06863135. In some embodiments, a cycle is 28 days unless the patient is on a Q3W dosing frequency of PF06863135. In some embodiments, a cycle is 21 days in cycle 1 and until the end of the last cycle when the subject is on the first treatment dosing.

Also provided are methods of treating cancer, comprising administering elranatamab (PF06863135) to a subject according a dosing schedule as shown below, and wherein the dosing schedule is described by a week number, a dose amount and a dose frequency corresponding to each week number:

| | | |
|---|---|---|
| Week Number | Dose Amount (mg) | Dose Frequency |

(a)

| | | |
|---|---|---|
| 1 | 44; 32; 12 plus 32; or A | Weekly |
| | plus B; | |
| 2-24; | 32; 44; 76; 116; or 152 | Weekly |
| 25 onwards; | 32; 44; 76; 116; or 152 | Weekly; very two weeks; every |
| | | three weeks; or every four |
| | | weeks |

(b)

| | | |
|---|---|---|
| 1 | 44; 32; 12 plus 32; or A | Weekly |
| | plus B; | |
| 2-25; | 32; 44; 76; 116; or 152 | Weekly |
| 26 onwards; | 32; 44; 76; 116; or 152 | Weekly; very two weeks; every |
| | | three weeks; or every four |
| | | weeks |

(c)

| | | |
|---|---|---|
| 1 | 44; 32; 12 plus 32; or A | Weekly |
| | plus B; | |
| 2-26; | 44; or 76 | Weekly |
| 27 onwards; | 44; or 76 | Weekly; very two weeks; every |
| | | three weeks; or every four |
| | | weeks |

(d)

| | | |
|---|---|---|
| 1 | 44; 32; 12 plus 32; or A | Weekly |
| | plus B; | |
| 2-24 | 32; 44; 76; 116; or 152 | Every two weeks |
| 25 onwards | 32; 44; 76; 116; or 152 | Every two weeks; every three |
| | | weeks; or every four weeks |

(e)

| | | |
|---|---|---|
| 1 | 44; 32; 12 plus 32; or A | Weekly |
| | plus B; | |
| 2-25 | 32; 44; 76; 116; or 152 | Every two weeks |
| 26 onwards | 32; 44; 76; 116; or 152 | Every two weeks; every three |
| | | weeks; or every four weeks |

or (f)

| | | |
|---|---|---|
| 1 | 44; 32; 12 plus 32; or A | Weekly |
| | plus B; | |
| 2-26 | 32; 44; 76; 116; or 152 | Every two weeks |
| 27 onwards | 32; 44; 76; 116; or 152 | Every two weeks; every three |
| | | weeks; or every four weeks |

wherein when the dose amount is 12 mg plus 32 mg during week 1, the dose amount of 12 mg is administered on one day, subsequently, the dose amount of 32 mg is administered on another day, wherein A plus B is 4 (A) plus 20 (B), 8 (A) plus 16 (B), 12 (A) plus 12 (B), or 8 (A) plus 24 (B), and wherein when the dose amount is A mg plus B mg during week 1, the dose amount of A mg is administered on one day, subsequently, the dose amount of B mg is administered on another day.

In come embodiments, the subject is administered elranatamab (PF06863135) according to the dosing schedule as shown below,

| | | |
|---|---|---|
| Week Number | Dose Amount (mg) | Dose Frequency |

(a)

| | | |
|---|---|---|
| 1 | 44 | Weekly |
| 2-24 | 76 | Weekly |
| 25 onwards | 76 | Weekly; every two weeks; |
| | | every three weeks; or every |
| | | four weeks |

(b)

| | | |
|---|---|---|
| 1 | 44 | Weekly |
| 2-25 | 76 | Weekly |
| 26 onwards | 76 | Weekly; every two weeks; |
| | | every three weeks; or every |
| | | four weeks |

(c)

| | | |
|---|---|---|
| 1 | 44 | Weekly |
| 2-26 | 76 | Weekly |
| 27 onwards | 76 | Weekly; every two weeks; |
| | | every three weeks; or every |
| | | four weeks |

(d)

| | | |
|---|---|---|
| 1 | 44 | Weekly |
| 2-24 | 76 | Every two weeks |
| 25 onwards | 76 | Every two weeks; every three |
| | | weeks; or every four weeks |

(e)

| | | |
|---|---|---|
| 1 | 44 | Weekly |
| 2-25 | 76 | Every two weeks |
| 26 onwards | 76 | Every two weeks; every three |
| | | weeks; or every four weeks |

or (f)

| | | |
|---|---|---|
| 1 | 44 | Weekly |
| 2-26 | 76 | Every two weeks |
| 27 onwards | 76 | Every two weeks; every three |
| | | weeks; or every four weeks |

In some embodiments, the subject is administered PF06863135 according to the dosing schedule (a), (b) or (c), and the dose frequency for week 25 onwards, week 26 onwards, and week 27 onwards in the dosing schedule (a), (b), and (c), respectively, is (i) weekly, (ii) every two weeks, (iii) every three weeks; (iv) every four weeks; (v) weekly or every two weeks; (vi) weekly or every three weeks, or (vii) weekly or every four weeks. In some embodiments, the subject is administered PF06863135 according to the dosing schedule (d), (e) or (f), and the dose frequency for week 25 onwards, week 26 onwards, and week 27 onwards in the dosing schedule (d), (e), and (f), respectively, is (i) every two weeks, (ii) every three weeks, (iii) very four weeks, (iv) every two weeks or every three weeks, or (v) every two weeks or every four weeks.

In some embodiments, the subject is administered elranatamab (PF06863135) according the dosing schedule as shown below,

| | | |
|---|---|---|
| Week Number | Dose Amount (mg) | Dose Frequency |

(a)

| | | |
|---|---|---|
| 1 | 12 plus 32 | Weekly |
| 2-24 | 76 | Weekly |
| 25 onwards | 76 | Weekly; every two weeks; |
| | | every three weeks; or every |
| | | four weeks |

(b)

| | | |
|---|---|---|
| 1 | 12 plus 32 | Weekly |
| 2-25 | 76 | Weekly |
| 26 onwards | 76 | Weekly; every two weeks; |
| | | every three weeks; or every |
| | | four weeks |

(c)

| | | |
|---|---|---|
| 1 | 12 plus 32 | Weekly |
| 2-26 | 76 | Weekly |
| 27 onwards | 76 | Weekly; every two weeks; |
| | | every three weeks; or every |
| | | four weeks |

(d)

| | | |
|---|---|---|
| 1 | 12 plus 32 | Weekly |
| 2-24 | 76 | Every two weeks |
| 25 onwards | 76 | Every two weeks; every three |
| | | weeks; or every four weeks |

(e)

| | | |
|---|---|---|
| 1 | 12 plus 32 | Weekly |
| 2-25 | 76 | Every two weeks |
| 26 onwards | 76 | Every two weeks; every three |
| | | weeks; or every four weeks |

or (f)

| | | |
|---|---|---|
| 1 | 12 plus 32 | Weekly |
| 2-26 | 76 | Every two weeks |
| 27 onwards | 76 | Every two weeks; every three |
| | | weeks; or every four weeks |

In some embodiments, the subject is administered 12 mg of elranatamab on day 1 of week 1, followed by 32 mg of elranatamab on day 4 of week 1. In some embodiments, the subject is administered PF06863135 according to the dosing schedule (a), (b) or (c), and the dose frequency for week 25 onwards, week 26 onwards, and week 27 onwards in the dosing schedule (a), (b), and (c), respectively, is (i) weekly, (ii) every two weeks, (iii) every three weeks; (iv) every four weeks; (v) weekly or every two weeks; (vi) weekly or every three weeks, or (vii) weekly or every four weeks. In some embodiments, the subject is administered PF06863135 according to the dosing schedule (d), (e) or (f), and the dose frequency for week 25 onwards, week 26 onwards, and week 27 onwards in the dosing schedule (d), (e), and (f), respectively, is (i) every two weeks, (ii) every three weeks, (iii) very four weeks, (iv) every two weeks or every three weeks, or (v) every two weeks or every four weeks.

In some embodiments, the subject is administered elranatamab (PF06863135) according the dosing schedule as shown below,

| | | |
|---|---|---|
| Week Number | Dose Amount (mg) | Dose Frequency |

(a)

| | | |
|---|---|---|
| 1 | 32; or 12 plus 32 | Weekly |
| 2-24 | 44 | Weekly |
| 25 onwards | 44 | Weekly; every two weeks; |
| | | every three weeks; or every |
| | | four weeks |

(b)

| | | |
|---|---|---|
| 1 | 32; or 12 plus 32 | Weekly |
| 2-25 | 44 | Weekly |
| 26 onwards | 44 | Weekly; every two weeks; |
| | | every three weeks; or every |
| | | four weeks |

(c)

| | | |
|---|---|---|
| 1 | 32; or 12 plus 32 | Weekly |
| 2-26 | 44 | Weekly |
| 27 onwards | 44 | Weekly; every two weeks; |
| | | every three weeks; or every |
| | | four weeks |

(d)

| | | |
|---|---|---|
| 1 | 32; or 12 plus 32 | Weekly |
| 2-24 | 44 | Every two weeks |
| 25 onwards | 44 | Every two weeks; every three |
| | | weeks; or every four weeks |

(e)

| | | |
|---|---|---|
| 1 | 32; or 12 plus 32 | Weekly |
| 2-25 | 44 | Every two weeks |
| 26 onwards | 44 | Every two weeks; every three |
| | | weeks; or every four weeks |

or (f)

| | | |
|---|---|---|
| 1 | 32; or 12 plus 32 | Weekly |
| 2-26 | 44 | Every two weeks |
| 27 onwards | 44 | Every two weeks; every three |
| | | weeks; or every four weeks |

In some embodiments, the subject is administered a single dose of 32 mg of elranatamab during week 1. In some embodiments, the subject is administered 12 mg of elranatamab on day 1 of week 1, followed by 32 mg of elranatamab on day 4 of week 1. In some embodiments, the subject is administered PF06863135 according to the dosing schedule (a), (b) or (c), and the dose frequency for week 25 onwards, week 26 onwards, and week 27 onwards in the dosing schedule (a), (b), and (c), respectively, is (i) weekly, (ii) every two weeks, (iii) every three weeks; (iv) every four weeks; (v) weekly or every two weeks; (vi) weekly or every three weeks, or (vii) weekly or every four weeks. In some embodiments, the subject is administered PF06863135 according to the dosing schedule (d), (e) or (f), and the dose frequency for week 25 onwards, week 26 onwards, and week 27 onwards in the dosing schedule (d), (e), and (f), respectively, is (i) every two weeks, (ii) every three weeks, (iii) very four weeks, (iv) every two weeks or every three weeks, or (v) every two weeks or every four weeks.

In some embodiments, wherein the dose amount and the dose frequency during week 1 are together referred to as a priming dosing, and if the subject is administered only one dose of elranatamab in the priming dosing, such one dose is referred to as a single priming dose, if the subject is sequentially administered two doses of elranatamab during week 1, the two doses are referred to as a first priming dose and a second priming dose respectively; the dose amount and dose frequency during weeks 2-24, weeks 2-25 and weeks 2-26 in the respective dosing schedules (a) and (d), (b) and (e) and (c) and (f), respectively, are in each dosing schedule together referred to as a first treatment dosing, the dose amount and the dose frequency during week 25 and onwards, week 26 onwards, and week 27 onwards in the respective dosing schedules (a) and (d), (b) and (e) and (c) and (f), are in each dosing schedule together referred to as a second treatment dosing.

In some embodiments, the subject is administered PF06863135 of the second treatment dosing for 6 to 18 cycles, thereafter, the subject is administered a third treatment dosing of PF06863135 subcutaneously, wherein the third treatment dosing is 32 mg Q2W, 32 mg Q4W, 44 mg Q2W, 44 mg Q4W, 76 mg Q2W, 76 mg Q4W, 116 mg Q2W, 116 mg Q4W, 152 mg Q2W, or 152 mg Q4W, wherein a cycle is 21 days or 28 days, and cycle 1 starts on day 1 week 1, day 1 week 2 or day 1 week 3.

In some embodiments, the first treatment dosing is 32 mg Q1W, the second treatment dosing is 32 mg Q1W or 32 mg Q2W and the third treatment dosing is 32 mg Q2W or 32 mg Q4W. In some embodiments, the first treatment dosing is 32 mg Q1W, the second treatment dosing is 32 mg Q2W and the third treatment dosing is 32 mg Q4W. In some embodiments, the first treatment dosing is 44 mg Q1W, the second treatment dosing is 44 mg Q1W or 44 mg Q2W and the third treatment dosing is 44 mg Q2W or 44 mg Q4W. In some embodiments, the first treatment dosing is 44 mg Q1W, the second treatment dosing is 44 mg Q2W and the third treatment dosing is 44 mg Q4W. In some embodiments, the first treatment dosing is 76 mg Q1W, the second treatment dosing is 76 mg Q1W or 76 mg Q2W and the third treatment dosing is 76 mg Q2W or 76 mg Q4W. In some embodiments, the first treatment dosing is 76 mg Q1W, the second treatment dosing is 76 mg Q2W and the third treatment dosing is 76 mg Q4W. In some embodiments, the first treatment dosing is 116 mg Q1W, the second treatment dosing is 116 mg Q1W or 116 mg Q2W and the third treatment dosing is 116 mg Q2W or 116 mg Q4W. In some embodiments, the first treatment dosing is 116 mg Q1W, the second treatment dosing is 116 mg Q2W and the third treatment dosing is 116 mg Q4W. In some embodiments, the first treatment dosing is 152 mg Q1W, the second treatment dosing is 152 mg Q1W or 32 mg Q2W and the third treatment dosing is 152 mg Q2W or 152 mg Q4W.

Also provided are methods of treating cancer, comprising administering elranatamab (PF06863135) to a subject according to a dosing schedule as shown below, and wherein the dosing schedule is described by a week number, a dose amount and a dose frequency corresponding to each week number:

| Week Number | Dose Amount (mg) | Dose Frequency |
|---|---|---|
| 1 | 44; or 32; or 12 plus 32; or | Weekly |
| | A plus B | |
| 2-4 | 44 to 152; | Weekly |
| 5-24 | 44 to 152 | Weekly; or every two weeks |
| 25 onwards | 44 to 152 | Every two weeks; every three |
| | | weeks or every four weeks |

wherein when the dose amount is 12 mg plus 32 mg during week 1, the dose amount of 12 mg is administered on one day, subsequently, the dose amount of 32 mg is administered on another day, wherein A plus B is 4 (A) plus 20 (B), 8 (A) plus 16 (B), 12 (A) plus 12 (B), or 8 (A) plus 24 (B), and wherein the dose amount of A mg is administered on one day, subsequently, the dose amount of B mg is administered on another day.

In some embodiments, the subject is administered 12 mg of Elranatamab on day 1 of week 1, followed by 32 mg of elranatamab on day 4 of week 1.

In some embodiments, the subject is administered elranatamab according the following dosing schedule

| Week Number | Dose Amount (mg) | Dose Frequency |
|---|---|---|
| 1 | 12 plus 32 | Weekly |
| 2-4 | 76 | Weekly |
| 5-24 | 116 | Every two weeks |
| 25 onwards | 116 | Every two weeks; every three |
| | | weeks; or every four weeks |

In some embodiments, the dose frequency during week 25 onwards, the dose frequency is every four weeks.

In some embodiments, the subject is administered elranatamab according the following dosing schedule

| Week Number | Dose Amount (mg) | Dose Frequency |
|---|---|---|
| 1 | 12 plus 32 | Weekly |
| 2-4 | 76 | Weekly |
| 5-24 | 152 | Every two weeks |
| 25 onwards | 152 | Every two weeks; every three |
| | | weeks; or every four weeks |

In some embodiments, the dose frequency during week 25 onwards is every four weeks.

In some embodiments, the dose amount and the dose frequency during week 1 are together referred to as a priming dosing, and if the subject is administered only one dose of elranatamab in the priming dosing, such one dose is referred to as a single priming dose, if the subject is sequentially administered two doses of elranatamab during week 1, the two doses are referred to as a first priming dose and a second priming dose respectively, the dose amount and dose frequency during weeks 2-4 are together referred to as a first treatment dosing, the dose amount and the dose frequency during weeks 5-24 and are together referred to as a second treatment dosing, and the dose amount and dose frequency during week 25 and onwards are together referred to as a third treatment dosing.

Also provided are methods of treating cancer, comprising administering elranatamab (PF06863135) to a subject according to a dosing schedule as shown below, and wherein the dosing schedule is described by a week number, a dose amount and a dose frequency corresponding to each week number:

| Week Number | Dose Amount (mg) | Dose Frequency |
|---|---|---|
| 1 | 44; or 32; or 12 plus 32; or | Weekly |
| | A plus B | |
| 2-4 | 44 to 76 | Weekly |
| 5-12 | 44 to 152; | Weekly |
| 13-24 | 44 to 152 | Weekly; or every two weeks |
| 25 onwards | 44 to 152 | Every two weeks; every three |
| | | weeks; or every four weeks |

wherein when the dose amount is 12 mg plus 32 mg during week 1, the dose amount of 12 mg is administered on one day, subsequently, the dose amount of 32 mg is administered on another day, wherein A plus B is 4 (A) plus 20 (B), 8 (A) plus 16 (B), 12 (A) plus 12 (B), or 8 (A) plus 24 (B), and wherein the dose amount of A mg is administered on one day, subsequently, the dose amount of B mg is administered on another day. In some embodiments, the subject is administered 12 mg of Elranatamab on day 1 of week 1, followed by 32 mg of elranatamab on day 4 of week 1.

In some embodiments, the subject is administered elranatamab according the following dosing schedule

| Week Number | Dose Amount (mg) | Dose Frequency |
|---|---|---|
| 1 | 12 plus 32 | Weekly |
| 2 to 4 | 76 | Weekly |
| 5-12 | 116 | Weekly |
| 13-24 | 116 | Weekly; or every two weeks |
| 25 onwards | 116 | Every two weeks; every three |
| | | weeks; or every four weeks |

In some embodiments, the dose frequency during weeks 13-24 is every two weeks. In some embodiments, the dose frequency during week 25 and onward is every four weeks.

In some embodiments, the subject is administered elranatamab according the following dosing schedule

| Week Number | Dose Amount (mg) | Dose Frequency |
|---|---|---|
| 1 | 12 plus 32 | Weekly |
| 2-4 | 76 | weekly |
| 2-12 | 152 | Weekly |
| 13-24 | 152 | Weekly or Every two weeks |
| 25 onwards | 152 | Every two weeks; or every four |
| | | weeks |

In some embodiments, the dose frequency during week 13-24 is every two weeks. In some embodiments, the dose frequency during week 25 onward is every four weeks.

In some embodiments, the subject is administered elranatamab according the following dosing schedule

| Week Number | Dose Amount (mg) | Dose Frequency |
|---|---|---|
| 1 | 12 plus 32 | Weekly |
| 2-4 | 76 | weekly |
| 5-12 | 76 | Weekly |
| 13-24 | 76 | Weekly; or every two weeks |
| 25 onwards | 76 | Every two weeks; every three |
| | | weeks; or every four weeks |

In some embodiments, the dose frequency during weeks 13-24 is every two weeks. In some embodiments, the dose frequency during week 25 and onward is every four weeks. In some embodiments, the dose frequency during weeks 13-24 is every two weeks, wherein the dose frequency during week 25 onward is every two weeks or every four weeks.

In some embodiments, the subject is administered elranatamab according the following dosing schedule

| Week Number | Dose Amount (mg) | Dose Frequency |
|---|---|---|
| 1 | 12 plus 32 | Weekly |
| 2-4 | 44 | weekly |
| 5-12 | 44 | Weekly |
| 13-24 | 44 | Weekly or Every two weeks |
| 25 onwards | 44 | Every two weeks; every three |
| | | weeks; or every four weeks |

In some embodiments, the dose frequency during week 13-24 is every two weeks. In some embodiments, the dose frequency during week 25 onwards is every four weeks.

In some embodiments, the dose amount and the dose frequency during week 1 are together referred to as a priming dosing, and if the subject is administered only one dose of elranatamab in the priming dosing, such one dose is referred to as a single priming dose, if the subject is sequentially administered two doses of elranatamab during week 1, the two doses are referred to as a first priming dose and a second priming dose respectively, the dose amount and dose frequency during weeks 2-4 and the dose amount and dose frequency during weeks 5-12 are all together referred to as a first treatment dosing, the dose amount and the dose frequency during weeks 13-24 and are together referred to as a second treatment dosing, and the dose amount and dose frequency during week 25 onwards are together referred to as a third treatment dosing.

The present invention is further directed to elranatamab (PF-06853135) for use in a method of treating cancer with a dosing regimen as defined herein.

In some embodiments, the cancer is multiple myeloma. In some embodiments, the cancer is advanced multiple myeloma. In some embodiments, the cancer is relapsed or refractory multiple myeloma.

In some embodiments, the cancer is triple class refractory multiple myeloma. In some embodiments, the multiple myeloma of the subject is refractory to all three types of the following multiple myeloma therapies (1) a prior multiple myeloma therapy that comprises a proteasome inhibitor, (2) a prior multiple myeloma therapy that comprises an immunomodulatory agent and (3) a prior multiple myeloma therapy that comprises an anti-CD38 antibody.

In some embodiments, the cancer is double class refractory multiple myeloma. In some embodiments, the multiple myeloma of the subject is refractory to at least two of the following three types multiple myeloma therapies (1) a prior multiple myeloma therapy that comprises a proteasome inhibitor, (2) a prior multiple myeloma therapy that comprises an immunomodulatory agent and (3) a prior multiple myeloma therapy that comprises an anti-CD38 antibody.

In some embodiments, the cancer is newly diagnosed multiple myeloma. In some embodiments, the cancer is multiple myeloma, and the subject has received stem cell transplant. In some embodiments, the subject has received autologous stem cell transplant. In some embodiments, the subject has received autologous stem cell transplant or allogeneic stem cell transplant. In some embodiments, the subject is minimum residual disease positive post stem cell transplant.

In some embodiments, the cancer is multiple myeloma, wherein in some embodiments the subject has progressed or is intolerant of an established multiple myeloma therapy. In some embodiments, the established multiple myeloma therapy comprises at least one drug selected from the group consisting of a proteasome inhibitor, an IMid drug and an anti-CD38 antibody.

In some embodiments, the cancer is multiple myeloma wherein the subject has received at least four prior therapies and the subject's multiple myeloma is refractory or relapsed to (1) a prior multiple myeloma therapy that comprises an proteasome inhibitor, (2) a prior multiple myeloma therapy that comprises an immunomodulatory agent and (3) a prior multiple myeloma therapy that comprises an anti-CD38 monoclonal antibody, and wherein the subject has demonstrated disease progression on the last therapy. In one aspect of these embodiments, the subject has received a prior therapy of a BCMA targeted ADC or a BCMA targeted CAR-T. In another aspect of these embodiments, the subject has not received any prior therapy of a BCMA targeted ADC or a BCMA targeted CAR-T.

In some embodiments, the cancer is multiple myeloma, the subject has received at least one, at least two, at least three or at least four prior multiple myeloma therapies, and the subject's multiple myeloma is refractory or relapsed to (1) a prior multiple myeloma therapy that comprises a proteasome inhibitor, (2) a prior multiple myeloma therapy that comprises an immunomodulatory agent and (3) a prior multiple myeloma therapy that comprises an anti-CD38 antibody, and the subject has demonstrated disease progression on the last multiple myeloma therapy. In one aspect of this embodiment, the subject has received at least three prior multiple myeloma therapies. In another aspect of this embodiment, the subject has received at least four prior multiple myeloma therapies.

In some embodiments, the previous multiple myeloma therapies the subject received comprise a BCMA directed ADC therapy or a BCMA directed CAR-T cell therapy. In some embodiments, the previous multiple myeloma therapies the subject received comprise a BCMA directed therapy.

In some embodiments, the previous multiple myeloma therapies the subject received do not comprise a BCMA directed ADC therapy or a BCMA directed CAR-T cell therapy. In some embodiments, the previous multiple myeloma therapies the subject received do not comprise a BCMA directed therapy.

In some embodiments, the cancer is multiple myeloma, and the subject has received at least one or at least two prior multiple myeloma therapies, the subject's multiple myeloma is refractory or relapsed to (1) a prior multiple myeloma therapy that comprises a proteasome inhibitor and (2) a prior multiple myeloma therapy that comprises an immunomodulatory agent. In some embodiments, the subject has demonstrated disease progression on the last multiple myeloma therapy.

In some embodiments, the cancer is multiple myeloma, and the subject has not received any prior multiple myeloma therapies. In some embodiments, the subject has not received any prior multiple myeloma therapies after the diagnosis of multiple myeloma. In some embodiments, the subject is stem cell transplant ineligible. In some embodiments, the cancer is multiple myeloma and the subject is stem cell transplant ineligible. In some embodiments, the subject is autologous stem cell transplant ineligible. In some embodiments, the subject is allogeneic stem cell transplant ineligible. In some embodiments, the subject is ineligible for autologous stem cell transplant and is also ineligible for allogeneic stem cell transplant.

In some embodiments, (i) a cycle is 21 days when the subject is on weekly or every three weeks dose frequency of PF06863135, a cycle is 28 days when the subject is on an every two weeks or every four weeks dose frequency of PF06863135; or (ii) a cycle is 28 days unless the patient is on a every three week dose frequency of PF06863135.

In some embodiments, the method further comprising administering sasanlimab to the subject.

In some embodiments, both PF-06863135 and sasanlimab are administered in a treatment cycle of four weeks, for at least a first treatment cycle, and wherein if a priming dosing of PF-06863135 is administered, the first treatment cycle starts on the seventh day after the single priming dose or the last dose of the priming dosing is administered, and sasanlimab is administered at a dose of 300 mg Q4W SC.

In some embodiments, wherein the first dose of sasanlimab is administered on day one of the first treatment cycle. In some embodiments, the first dose of PF-06863135 in a treatment cycle is administered on day 1 of the treatment cycle.

In some embodiments, week 1 and cycle 1 starts on the day when the single priming dose or the first priming dose is administered to the subject, or if the subject is not administered a priming dosing or priming dose of PF06863135, week 1 and cycle 1 starts on the day when a first dose in the first treatment dosing of PF06863135 is administered to the subject, a cycle is 28 days, and sasanlimab is administered at a dose of 300 mg Q4W SC. In some embodiments, the subject is administered at least one priming dose of PF6863135, and sasanlimab is administered to the subject on day 8 of each cycle.

In some embodiments, the method further comprising administering lenalidomide to the subject.

In some embodiments, both PF-06863135 and lenalidomide are administered in a treatment cycle of four weeks, for at least a first treatment cycle, and wherein if a priming dosing of PF-06863135 is administered, the first treatment cycle starts on the seventh day after the single priming dose or the last dose of the priming dosing is administered, and wherein lenalidomide is administered at a dose of 25 mg daily orally on day 1 to day 21 of each treatment cycle.

In some embodiments, lenalidomide is administered at a dose of 25 mg daily orally on day 1 to day 21 of each treatment cycle without dexamethasone.

In some embodiments, the first dose of PF-06863135 in a treatment cycle is administered on day 1 of the treatment cycle.

In some embodiments, a priming dosing of PF6863135 is administered, a cycle is 28 days, lenalidomide is administered at a daily oral dose of about 5 mg, about 10 mg, about 15 mg, about 20 mg or about 25 mg on day 8-28 or day 15-28 of the first cycle, and on day 1-28 of the second and third cycle, afterwards, starting on the fourth cycle, lenalidomide is administered at a daily oral dose of about 5 to 10 mg higher than that is administered during the third cycle, or continued to be administered at the same daily oral dose as that of the third cycle on day 1-28 of each cycle.

In some embodiments, a priming dosing of PF06863135 is administered, lenalidomide is administered at a daily oral dose of about 10 mg, or about 15 mg starting on day 8 of cycle 1 for at least 10 consecutive days in each cycle.

In some embodiments, no priming dosing of PF06863135 is administered, and lenalidomide is administered at a daily oral dose of about 10 mg, about 15 mg, about 20 mg or about 25 mg, for at least 10, at least 14 or at least 21 consecutive days, in each cycle.

In some embodiments, the subject is administered PF06863135 in an induction phase followed by a maintenance phase, wherein the induction phase starts on the day when the first dose in the priming dosing of PF06863135 is administered, or if no priming dosing of PF06863135 is administered then the induction phase starts on the day when the first dose in the first treatment dosing of PF06863135 is administered, and the induction phase ends on the last day of the last week, or the last day of the last cycle, whichever is later, when the subject is on the first treatment dosing; wherein during the induction phase, lenalidomide is administered at a lenalidomide induction dosing of a daily oral dose of about 5 mg to about 25 mg during at least 10 consecutive days in each cycle in the induction phase; in the maintenance phase, PF06863135 is administered in the second treatment dosing, lenalidomide is administered on a lenalidomide maintenance dosing of an oral daily dose of about 5 mg to about 25 mg for at least 10 consecutive days in a cycle; wherein each cycle is 21 days or 28 days, and the induction phase lasts 1 to 10 cycles. In some embodiments, the method further comprising administering to the subject dexamethasone during the induction phase at a dexamethasone dosing of about 10 mg to about 40 mg daily oral on at least day 1 and day 8 of the first cycle in the induction phase.

In some embodiments, each cycle in the induction phase is 21 days or 28 days, and cycle 1 starts on day 1 week 3, the lenalidomide induction dosing is about 5 mg, about 10 mg, about 15 mg, about 20 mg or about 25 mg daily oral and is administered on day 1 to day 14, or day 1-21 in each cycle in the induction phase, and if dexamethasone is administered, it is administered at a dosing of about 20 mg daily on day 1, 8, and 15 in the first cycle and the second cycle of the induction phase; wherein each cycle in the maintenance phase is 28 days, and the maintenance lenalidomide dosing is about 5 mg, about 10 mg, or about 15 mg oral daily on day 1 to day 28 of each cycle in the maintenance phase. In some embodiments, the induction phase ends after 24-26 weeks. In some embodiments, the induction phase ends after 12-14 weeks.

In some embodiments, the method further comprising administering pomalidomide to the subject. In some embodiments, both PF06863135 and pomalidomide are administered in a treatment cycle of four weeks, for at least a first treatment cycle, and wherein a priming dosing of PF-06863135 is administered, the first treatment cycle starts on the seventh day after the single priming dose or the last dose of the priming dosing is administered, pomalidomide is administered at a dose of 4 mg daily orally on day 1 to day 21 of each treatment cycle. In some embodiments, pomalidomide is administered at a dose of 4 mg daily, 3 mg daily, 2 mg daily or 1 mg daily orally on day 1 to day 21 of each treatment cycle without dexamethasone. In some embodiments, the first dose of PF-06863135 in a treatment cycle is administered on day 1 of the treatment cycle.

In some embodiments, the method further comprising administering daratumumab to the subject. In some embodiments, daratumumab is administered subcutaneously at a daratumumab dosing of about 1800 mg weekly, every two weeks, every three weeks, or every four weeks. In some embodiments, daratumumab dosing starts as about 1800 weekly in cycle 1 for about 8 doses, followed by about 1800 mg every two weeks for about 8 to about 10 doses, followed by about 1800 mg every four weeks thereafter.

In some embodiments, the method further comprising administering to the subject isatuximab. In some embodiments, isatuximab is administered at an isatuximab dosing of about 5 mg to about 10 mg/kg QW IV, Q2W IV, Q3W IV or Q4W IV. In some embodiments, the isatuximab dosing under which that the isatuximab administered to the subject can be the same or different while the subject is on the priming dosing, the first treatment dosing, the second treatment dosing or the third treatment dosing of PF06863135.

In some embodiments, the method further comprising administering to the subject at least one dose of a premedication on the day when the single priming dose, the first priming dose, the second priming dose or the first dose of the first treatment dose of PF06863135 is administered to the subject, wherein the premeditation is acetaminophen, diphenhydramine or dexamethasone. In some embodiments, dexamethasone is administered at a dexamethasone dosing of about 10 mg to about 40 mg daily oral or intravenous. In some embodiments, dexamethasone is administered at a dexamethasone dosing of about 10 mg to about 40 mg daily, oral, or intravenous, at least on the day when the first dose of the first treatment dosing of PF06863135 is administered to the subject. In some embodiments, the dexamethasone dosing under which the dexamethasone is administered to the subject as a premedication can be the same or different while the subject is on the priming dosing, the first treatment dosing, the second treatment dosing or the third treatment dosing of PF06863135.

In some embodiments, the method further comprising administering to the subject a second therapeutic agent. In some embodiments, the second therapeutic agent is an anticancer agent. In some embodiment, the second therapeutic agent is an GSI. In some embodiment, the second therapeutic agent is nirogacestat or a pharmaceutically acceptable salt thereof.

In some embodiments, the method further comprising administering to the subject radiotherapy.

In aspects and/or embodiments that refer to a method of treatment as described herein, such aspects and/or embodiments are also further aspects and/or embodiments concerning the therapeutic agent or agents for use in that method of treatment, or alternatively the use of the defined therapeutic agent or agents for use in the manufacture of a medicament or medicaments for use in that treatment.

### BRIEF DESCRIPTION OF THE FIGURES/DRAWINGS

FIG. 1 depicts the inducement of PD-1 expression on CD8+ T cells after treatment with BCMAxCD3 bispecific antibody.

FIGS. 2A and 2B depict the therapeutic activity of a BCMAxCD3 bispecific antibody in combination with an anti-PD1 antibody in A) an orthotopic MM.1S-Luc-PDL1 multiple myeloma model and B) a subcutaneous MM.1S-PD-L1 multiple myeloma model.

FIG. 3A-3E depict the upregulation of BCMA expression on the cell surface of multiple myeloma cells after treatment with GSI.

FIG. 4A-4E depict the upregulation of BCMA expression on the cell surface of multiple myeloma cells, in a time-dependent manner, after treatment with GSI.

FIG. 5A-5E depict the reduction of shedding of soluble BCMA (sBCMA) in multiple myeloma cells lines after treatment with GSI.

FIG. 6A-6E depict treatment with GSI improves BCMAxCD3 bispecific mediated cell killing in multiple myeloma cell lines.

FIG. 7A-7B depict the A) upregulation of BCMA expression on the cell surface of Raji lymphoma cells after treatment with GSI and B) upregulation is in a time-dependent manner.

FIG. 8 depicts treatment with GSI improves BCMAxCD3 bispecific mediated cell killing in a lymphoma cell line.

### DETAILED DESCRIPTION

The instant application relates to the treatment of cancer and/or cancer-associated disease. Certain aspects relate to the treatment of an individual having cancer or cancer-associated disease by administering to the individual a combination therapy of a first therapeutic that is a BCMAxCD3 bispecific antibody and second therapeutic that is an anti-PD-1 antibody, an anti-PD-L1 antibody or a γ-secretase inhibitor (GSI), or a pharmaceutically acceptable salt thereof.

### I. Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“About” when used to modify a numerically defined parameter (e.g., the dose of a BCMAxCD3 bispecific antibody, or the length of treatment time with a combination therapy described herein) means that the parameter may vary by as much as 10% below or above the stated numerical value for that parameter. For example, a dose of about 5 mg/kg may vary between 4.5 mg/kg and 5.5 mg/kg.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)2, Fv), single chain (scFv) and domain antibodies (including, for example, shark and camelid antibodies), and fusion proteins comprising an antibody, and any other modified configuration of the immunoglobulin molecule that comprises an antigen recognition site. An antibody includes an antibody of any class, such as IgG, IgA, or IgM (or subclass thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen binding fragment” or “antigen binding portion” of an antibody, as used herein, refers to one or more fragments of an intact antibody that retain the ability to specifically bind to a given antigen. Antigen binding functions of an antibody can be performed by fragments of an intact antibody. Examples of binding fragments encompassed within the term “antigen binding fragment” of an antibody include Fab; Fab′; F(ab′)2; an Fd fragment consisting of the VH and CH1 domains; an Fv fragment consisting of the VL and VH domains of a single arm of an antibody; a single domain antibody (dAb) fragment (Ward et al., Nature 341:544-546, 1989), and an isolated complementarity determining region (CDR).

A “bispecific antibody” or “dual-specific antibody” is a hybrid antibody having two different antigen binding sites. The two antigen binding sites of a bispecific antibody bind to two different epitopes, which may reside on the same or different protein targets.

A “B-cell maturation antigen bispecific antibody” or “BCMA bispecific antibody” is a bispecific antibody which specifically binds to BCMA and another antigen.

A “heterodimer,” “heterodimeric protein,” “heterodimeric complex,” or “heteromultimeric polypeptide” is a molecule comprising a first polypeptide and a second polypeptide, wherein the second polypeptide differs in amino acid sequence from the first polypeptide by at least one amino acid residue.

An antibody, a bispecific antibody, or a polypeptide that “preferentially binds” or “specifically binds” (used interchangeably herein) to a target (e.g., BCMA protein) is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody or bispecific antibody “specifically binds” or “preferentially binds” to a target if it binds with greater affinity, avidity, more readily, and/or with greater duration than it binds to other substances. For example, an antibody that specifically or preferentially binds to an BCMA epitope is an antibody that binds this epitope with greater affinity, avidity, more readily, and/or with greater duration than it binds to other BCMA epitopes or BCMA epitopes. It is also understood that by reading this definition, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

A “variable region” of an antibody refers to the variable region of the antibody light chain or the variable region of the antibody heavy chain, either alone or in combination. As known in the art, the variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-Iazikani et al., 1997, J. Molec. Biol. 273:927-948). As used herein, a CDR may refer to CDRs defined by either approach or by a combination of both approaches.

A “CDR” of a variable domain are amino acid residues within the variable region that are identified in accordance with the definitions of the Kabat, Chothia, the accumulation of both Kabat and Chothia, AbM, contact, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., Nature 342:877-883, 1989. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., J. Mol. Biol., 262:732-745, 1996. In another approach, referred to herein as the “conformational definition” of CDRs, the positions of the CDRs may be identified as the residues that make enthalpic contributions to antigen binding. See, e.g., Makabe et al., Journal of Biological Chemistry, 283:1156-1166, 2008. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given aspect containing more than one CDR, the CDRs may be defined in accordance with any of Kabat, Chothia, extended, AbM, contact, and/or conformational definitions.

“Isolated antibody” and “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

The terms “cancer”, “cancerous”, or “malignant” refer to or describe the physiological condition in mammals that is typically characterized by unregulated cell growth. A “cancer” or “cancer tissue” can include a tumor. Examples of cancer include but are not limited to, carcinoma, lymphoma, leukemia, myeloma, blastoma, and sarcoma. Cancers may include cancer and/or cancer-associated disease, including B-cell related cancers and/or cancer-associated diseases, including but not limited to, multiple myeloma, malignant plasma cell neoplasm, lymphoma, Hodgkin's lymphoma, nodular lymphocyte predominant Hodgkin's lymphoma, Kahler's disease and Myelomatosis, plasma cell leukemia, bony and extramedullary plasmacytoma with multiple myeloma, solid bony and extramedullary plasmacytoma, monoclonal gammopathy of unknown significance (MGUS), smoldering myeloma, light chain amyloidosis, osteosclerotic myeloma, B-cell prolymphocytic leukemia, hairy cell leukemia, B-cell non-Hodgkin's lymphoma (NHL), acute myeloid leukemia (AML), chronic lymphocytic leukemia (CLL), acute lymphocytic leukemia (ALL), chronic myeloid leukemia (CML), follicular lymphoma, Burkitt's lymphoma, marginal zone lymphoma, mantle cell lymphoma, large cell lymphoma, precursor B-lymphoblastic lymphoma, myeloid leukemia, Waldenstrom's macroglobulienemia, diffuse large B cell lymphoma, mucosa-associated lymphatic tissue lymphoma, small cell lymphocytic lymphoma, primary mediastinal (thymic) large B-cell lymphoma, lymphoplasmactyic lymphoma, marginal zone B cell lymphoma, splenic marginal zone lymphoma, intravascular large B-cell lymphoma, primary effusion lymphoma, lymphomatoid granulomatosis, T cell/histiocyte-rich large B-cell lymphoma, primary central nervous system lymphoma, primary cutaneous diffuse large B-cell lymphoma (leg type), EBV positive diffuse large B-cell lymphoma of the elderly, diffuse large B-cell lymphoma associated with inflammation, ALK-positive large B-cell lymphoma, plasmablastic lymphoma, large B-cell lymphoma arising in HHV8-associated multicentric Castleman disease, B-cell lymphoma unclassified with features intermediate between diffuse large B-cell lymphoma and Burkitt lymphoma, B-cell lymphoma unclassified with features intermediate between diffuse large B-cell lymphoma and classical Hodgkin lymphoma, and other B-cell related lymphoma. Examples of cancers and cancer-associated diseases are further described herein

“Chemotherapeutic agent” is a chemical compound useful in the treatment of cancer and/or cancer-associated disease. Classes of chemotherapeutic agents include, but are not limited to: alkylating agents, antimetabolites, kinase inhibitors, spindle poison plant alkaloids, cytotoxic/antitumor antibiotics, topoisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, and anti-sense oligonucleotides that inhibit expression of genes implicated in abnormal cell proliferation or tumor growth. Chemotherapeutic agents are further described herein.

“Chemotherapy” as used herein, refers to a chemotherapeutic agent, as defined above, or a combination of two, three or four chemotherapeutic agents, for the treatment of cancer and/or cancer-associated disease. When chemotherapy consists more than one chemotherapeutic agent, the chemotherapeutic agents can be administered to the patient on the same day or on different days in the same treatment cycle.

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition.

“Multiple myeloma therapy” refers to a drug, a combination of two or more drugs, (1) that is approved by United States Food and Drug Administration (USFDA) or European Medicines Agency for the treatment of multiple myeloma, or (2) that is or was in clinical trials in the US or Europe for the treatment of multiple myeloma.

“Established multiple myeloma therapy” refers to multiple myeloma therapy approved by USFDA or European Medicines Agency, which can be a drug, a combination therapy of two or more drugs.

An “IMiD drug”, “imid drug”, or an “immunomodulatory agent”, as used herein, interchangeably, refers to a drug that is understood by a practicing physician treating multiple myeloma as an IMiD drug or immunomodulatory agent in the context of the treatment of multiple myeloma. Examples of an IMid drug or an immunomodulating agent, includes, without limitation, thalidomide, lenalidomide and pomalidomide.

“BCMA directed ADC therapy”, refers to a multiple myeloma therapy that comprises an antibody drug conjugate, wherein the antibody binds to B-cell maturation antigen (BCMA). Examples of a BCMA directed ADC includes, without limitation, belantamab mafodotin -blmf, which was approved by USFDA and marketed under the brand name BLENREP.

“BCMA directed CAR-T cell therapy”, or “anti-BCMA CAR-T cell” as used herein, interchangeably, refers to a multiple myeloma therapy that comprises a chimeric antigen receptor T cell wherein the chimeric antigen receptor recognizes B-cell maturation antigen (BCMA). Examples of a “BCMA targeted CAR-T therapy”, or “anti-BCMA CAR T cell therapy” includes, without limitation, idecabtgene vicleucel (ide-cel; or bb2121) and JNJ-4528, also known as LCAR-B38M.

“BCMA directed therapy”, refers to a multiple myeloma therapy which active ingredient comprises a component that binds to the B-Cell maturation antigen. BCMA directed therapy includes BCMA directed ADC Therapy, BCMA directed CAR-T therapy, and multiple myeloma therapies that comprises BCMA bispecific antibodies.

“Newly diagnosed multiple myeloma” refers to multiple myeloma wherein the patient (subject) has not yet received any treatment for the diagnosis of multiple myeloma.

“Homology” refers to sequence similarity between two polypeptide sequences when they are optimally aligned. When a position in both of the two compared sequences is occupied by the same amino acid monomer subunit, e.g., if a position in a light chain CDR of two different Abs is occupied by alanine, then the two Abs are homologous at that position. The percent of homology is the number of homologous positions shared by the two sequences divided by the total number of positions compared×100. For example, if 8 of 10 of the positions in two sequences are matched or homologous when the sequences are optimally aligned then the two sequences are 80% homologous. Generally, the comparison is made when two sequences are aligned to give maximum percent homology. For example, the comparison can be performed by a BLAST algorithm wherein the parameters of the algorithm are selected to give the largest match between the respective sequences over the entire length of the respective reference sequences.

The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

“Patient”, “subject” or “individual” refers to any living organism suffering from or prone to a condition that can be prevented or treated by administration of a therapeutic agent or composition or combination as provided herein, such as a cancer and/or a cancer-associated disease, and includes both humans and animals. The terms “patients”, “subjects” and “individuals” include, but are not limited to, mammals (e.g., murines, simians, equines, bovines, porcines, canines, felines, and the like), and preferably are human.

“Sustained response” means a sustained therapeutic effect after cessation of treatment with a therapeutic agent, or a combination therapy described herein. In some aspects, the sustained response has a duration that is at least the same as the treatment duration, or at least 1.5, 2.0, 2.5 or 3 times longer than the treatment duration.

As used in herein, “administering” refers to the delivery of a therapeutic agent to a subject, using any of the various methods and delivery systems known to those skilled in the art. Exemplary routes of administration include intravenous, intramuscular, subcutaneous, intraperitoneal, spinal, or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent can be administered via a non-parenteral route, or orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

“Treat” or “treating” a cancer and/or a cancer-associated disease as used herein means to administer a combination therapy according to the present invention to a subject, patient or individual having a cancer, or diagnosed with a cancer, to achieve at least one positive therapeutic effect, such as, for example, reduced number of cancer cells, reduced tumor size, reduced rate of cancer cell infiltration into peripheral organs, or reduced rate of tumor metastasis or tumor growth, reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject. For the purposes of this invention, beneficial or desired clinical results include, but are not limited to, one or more of the following: reducing the proliferation of (or destroying) neoplastic or cancerous cell; inhibiting metastasis or neoplastic cells; shrinking or decreasing the size of tumor; remission of the cancer; decreasing symptoms resulting from the cancer; increasing the quality of life of those suffering from the cancer; decreasing the dose of other medications required to treat the cancer; delaying the progression the cancer; curing the cancer; overcoming one or more resistance mechanisms of the cancer; and/or prolonging survival of patients the cancer. Positive therapeutic effects in cancer can be measured in a number of ways (see, for example, W. A. Weber, J. Nucl. Med. 50:1S-10S (2009)). In some aspects, the treatment achieved by a combination of the invention is any of the partial response (PR), complete response (CR), overall response (OR), objective response rate (ORR), progression free survival (PFS), radiographic PFS, disease free survival (DFS) and overall survival (OS). PFS, also referred to as “Time to Tumor Progression” indicates the length of time during and after treatment that the cancer does not grow, and includes the amount of time patients have experienced a CR or PR, as well as the amount of time patients have experienced stable disease (SD). DFS refers to the length of time during and after treatment that the patient remains free of disease. OS refers to a prolongation in life expectancy as compared to naïve or untreated subjects or patients. In some aspects, response to a combination of the invention is any of PR, CR, PFS, DFS, ORR, OR or OS that is assessed using Response Evaluation Criteria in Solid Tumors (RECIST 1.1) response criteria (Eisenhauer et al., E. A. et al., Eur. J Cancer 45:228-247 (2009)). In some aspects, anti-myeloma activity may be evaluated by Overall response rate (ORR), time to response (TTR), complete response rate (CRR), duration of response (DOR), duration of complete response (DoCR), duration of stable disease (DOSD), progression-free survival (PFS), overall survival (OS), using International Myeloma Working Group (IMWG) criteria. The treatment regimen for a combination therapy as provided herein that is effective to treat a cancer patient may vary according to factors such as the disease state, age, and weight of the patient, and the ability of the therapy to elicit an anti-cancer response in the subject. While an aspect of any of the aspects of the invention may not be effective in achieving a positive therapeutic effect in every subject, it should do so in a statistically significant number of subjects as determined by any statistical test known in the art such as, but not limited to, the Cox log-rank test, the Cochran-Mantel-Haenszel log-rank test, the Student's t-test, the chi2-test, the U-test according to Mann and Whitney, the Kruskal-Wallis test (H-test), Jonckheere-Terpstrat-test and the Wilcon on-test. The term “treatment” also encompasses in vitro and ex vivo treatment, e.g., of a cell, by a reagent, diagnostic, binding compound, or by another cell.

As used herein, “pharmaceutical product” refers to a drug product that comprises an actively pharmaceutical ingredient and is regulated by the US FDA, EMA or other counterpart regulatory agencies in the other markets. A pharmaceutical product can be an investigational drug or a drug product that has already been approved by a regulatory agency.

The terms “treatment regimen”, “dosing protocol” and “dosing regimen” are used interchangeably to refer to the dose and timing of administration of each therapeutic agent in a combination of the invention.

As used herein, an “effective dosage” or “effective amount” of drug, compound, or pharmaceutical composition is an amount sufficient to affect any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing incidence or amelioration of one or more symptoms of various diseases or conditions (such as for example cancer), decreasing the dose of other medications required to treat the disease, enhancing the effect of another medication, and/or delaying the progression of the disease. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein “dosing” refers to both the “dose amount”, for example 1 mg, 20 mg, and the “dose frequency”, for example, once a day (QD), once a week (Q1W or QW), every two weeks (Q2W), every three weeks (Q3W) and every four weeks (Q4W). Dosing may also include the administration route of a drug, such as for example, subcutaneously (SC), intravenously (IV), oral (PO), if so specified. Similarly, a “priming dosing”, a “first treatment dosing”, a “second treatment dosing” and so on, each refers to both the dose amount and dose frequency of such dosing and optionally also includes the administration route if so specified. In some embodiments, there is one dose amount and one dose frequency in a dosing. In some embodiments, there are more than one dose amounts, and/or more than one dose frequencies in a dosing.

As used herein, “dose level”, unless otherwise specified, when used to describe the dose amount of elranatamab, (also known as PF06863135), refers to one of the following dose amounts: 4 mg, 8 mg, 12 mg, 16 mg, 20 mg, 24 mg, 32 mg, 44 mg, 76 mg, 116 mg and 152 mg, wherein 8 mg, 12 mg, 16 mg, 20 mg 24 mg, 32 mg, 44 mg, 76 mg, 116 mg and 152 mg are each one dose level higher than 4 mg, 8 mg, 12 mg, 16 mg, 24 mg, 32 mg, 44 mg, 76 mg, and 116 mg, respectively.

As used herein, a “respective regulatory label of the pharmaceutical product” means, an unexpired United States Prescribing Information (USPI) from US Food and Drug Administration (FDA), an unexpired Summary of Product Characteristics (SMPC) from European Medicine Agency (EMA), of the pharmaceutical product or similar labels of the pharmaceutical product from the regulatory agencies in other markets. In some embodiments, a “respective regulatory label of the pharmaceutical product” in a patent or patent application in the United States, refers to an unexpired USPI of the pharmaceutical product, and in a patent or patent application in an European country that adopts EMA marketing authorizations of the pharmaceutical product, an unexpired SMPC of the pharmaceutical product, and similarly in other jurisdictions.

As used herein the “subject's response”, refers to the clinical response of the subject being treated with the pharmaceutical product that comprises elranatamab (PF006863135) as monotherapy or in combination with a second therapeutic product, to the underlying treatment. The “subject's response” includes one or more aspects with regard to clinical efficacy, such as complete response, partial response and duration of the response. “Subject's response” may also include additional aspects such as toxicity and adverse events.

As used herein, “IMWG response” refers to a patient's (subject's) clinical response to a pharmaceutical product to treat multiple myeloma, wherein the response, such as a complete response, or partial response, is defined according to the most up to date definition from the International Myeloma Working Group.

As used herein, “cycle”, and “week” when used in the context of describing a method of treating cancer including uses thereof, a dosing, or a dosing schedule, refer to a duration of time. A cycle is 21 days or 28 days, unless otherwise specified, when a subject is treated with a therapeutic agent, a pharmaceutical product thereof, such as elranatamab (PF06863135), or a pharmaceutical product thereof, as a monotherapy or in combination with a second therapeutic agent. Week 1 refers to the first week when the subject is treated under the method, or any of the dosing or dosing schedules therein unless otherwise specified. Week 2 starts immediately after week 1 ends, week 3 starts immediately after week 2 ends, and so on. Cycle 1 starts on the first day of week 1, the first day of week 2, or the first day of week three, unless otherwise specified. Unless stated otherwise, cycle 2 starts immediately after cycle 1 ends, cycle 3 starts immediately after cycle 2 ends, and so on.

As used herein, “stem cell transplant ineligible” refers patient diagnosed with multiple myeloma being not eligible for stem cell transplant as a treatment for the multiple myeloma.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms). Multiple myeloma is a cancer of the plasma cells

“Tumor burden” also referred to as “tumor load”, refers to the total amount of tumor material distributed throughout the body. Tumor burden refers to the total number of cancer cells or the total size of tumor(s), throughout the body, including lymph nodes and bone narrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

The term “immunotherapy” refers to the treatment of a subject by a method comprising inducing, enhancing, suppressing, or otherwise modifying an immune response.

The term “immune effector cell” or “effector cell” as used herein refers to a cell within the natural repertoire of cells in the human immune system which can be activated to affect the viability of a target cell. The viability of a target cell can include cell survival, proliferation, and/or ability to interact with other cells.

“Pharmaceutically acceptable excipient” or “pharmaceutically acceptable carrier” refers to a component that may be included in the compositions described herein and causes no significant adverse toxicological effects to a subject.

The terms “protein”, “polypeptide” and “peptide” are used interchangeably herein and refer to any peptide-linked chain of amino acids, regardless of length co-translational or post-translational modification.

As used in herein, “substantially” or “essentially” means nearly totally or completely, for instance, 95% or greater of a given quantity.

The term “substantially homologous” or “substantially identical” means that a particular subject sequence, for example, a mutant sequence, varies from a reference sequence by one or more substitutions, deletions, or additions, the net effect of which does not result in an adverse functional dissimilarity between the reference and subject sequences. For purposes herein, a sequence having greater than 95 percent homology (identity), equivalent biological activity (although not necessarily equivalent strength of biological activity), and equivalent expression characteristics to a given sequence is considered to be substantially homologous (identical). For purposes of determining homology, truncation of the mature sequence should be disregarded.

The terms “synergy” or “synergistic” are used to mean that the result of the combination of two or more compounds, components or targeted agents is greater than the sum of each agent together. The terms “synergy” or “synergistic” also means that there is an improvement in the disease condition or disorder being treated, over the use of the two or more compounds, components or targeted agents while each compound, component or targeted agent individually. This improvement in the disease condition or disorder being treated is a “synergistic effect”. A “synergistic amount” is an amount of the combination of the two compounds, components or targeted agents that results in a synergistic effect, as “synergistic” is defined herein. Determining a synergistic interaction between one or two components, the optimum range for the effect and absolute dose ranges of each component for the effect may be definitively measured by administration of the components over different w/w (weight per weight) ratio ranges and doses to patients in need of treatment. However, the observation of synergy in in vitro models or in vivo models can be predictive of the effect in humans and other species and in vitro models or in vivo models exist, as described herein, to measure a synergistic effect and the results of such studies can also be used to predict effective dose and plasma concentration ratio ranges and the absolute doses and plasma concentrations required in humans and other species by the application of pharmacokinetic/pharmacodynamic methods.

As used herein, PF-06863135 is used interchangeably with elranatamab. PF06863135 is a BCMA×CD3 bispecific antibody. PF-06863135 is described, for example in U.S. Pat. No. 9,969,809. The selected sequences of PF-06863135 are shown in Table 15.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular.

Exemplary methods and materials are described herein, although methods and materials similar or equivalent to those described herein can also be used in the practice or testing of the invention. The materials, methods, and examples are illustrative only and not intended to be limiting.

### II. Methods, Uses and Medicaments

Provided herein are methods and compositions for treating a cancer and/or a cancer-associated disease in a subject that involves combination therapy which comprises at least a first therapeutic agent and a second therapeutic agent.

### BCMA-Specific Therapeutic Agents

In some aspects, the therapeutic agent may be a BCMA-specific therapeutic agent. In another aspect, the BCMA-specific therapeutic agent may be a BCMA multispecific antibody (e.g. bispecific and trispecific), a BCMA antibody-drug conjugate or a BCMA chimeric antigen receptor (CAR)-modified T cell therapy. B-cell maturation antigen (BCMA, also known as TNFRSF17 and CD269) is a candidate for bispecific antibody based immunotherapy. BCMA expression is upregulated during B-cell maturation into plasma blasts and plasma cells, but it is not expressed on naïve B cells, hematopoietic stem cells or normal tissues such as the heart, lung, kidney, or tonsil. In multiple myeloma, BCMA expression was identified at each disease stage, and on patients with differing cytogenetic risks. Furthermore, BCMA expression was not influenced by treatment with autologous stem cell transplant (ASCT) or chemotherapy. In vivo, bispecific antibodies against BCMA have been shown to induce T-cell activation, reduce tumor burden and prolong survival.

Examples of BCMA multispecific antibodies that may be useful in the combination therapies of the present invention include, but are not limited to, AMG 420 (BCMAxCD3 bispecific T-cell engager, BiTE®, Amgen), AMG 701 (BCMAxCD3 BiTE®, Amgen), CC-93269 (BCMAxCD3 bispecific antibody, Celgene), JNJ-64007957 (Janseen), PF-06863135 (BCMAxCD3 bispecific antibody, Pfizer Inc.), TNB-383B (TeneoBio/AbbVie), REGN5458 (BCMAxCD3 bispecific antibody, Regeneron), AFM26 (BCMAxCD16 tetravalent bispecific antibody, Affimed GmbH), HPN217 (BCMA×ALB×CD3 trispecific, Harpoon Therapeutics).

In some aspects, the BCMA-specific therapeutic agent is a BCMA bispecific antibody molecule. BCMA bispecific antibodies are monoclonal antibodies that have binding specificity for at least two different antigens (e.g. BCMA and CD3).

In some aspects, the BCMA bispecific antibody comprises a first antibody variable domain and a second antibody variable domain, wherein the first antibody variable domain specifically binds to CD3, and wherein the second antibody variable domain specifically binding to a BCMA.

In some aspects, a therapeutic agent in the combination therapy of the present invention is a BCMA bispecific antibody. In some aspects, a BCMA bispecific antibody may have any of the features or characteristics of any of the BCMA bispecific antibodies provided in WO2016166629, which is hereby incorporated by reference for all purposes.

In some aspects, the first antibody variable domain specifically binds to CD3. Information about CD3 is provided, for example, via UniProtKB #P07766. In some aspects, the first antibody variable domain comprises three CDRs of a heavy chain variable region (VH) comprising the amino acid sequence shown in SEQ ID NO: 1, and/or three CDRs of a light chain variable region (VL) comprising the amino acid sequence shown in SEQ ID NO: 9. In some aspects, the VH comprises a VH CDR1 comprising the sequence shown in SEQ ID NO: 2, 3 or 4, a VH CDR2 comprising the sequence shown in SEQ ID NO: 5 or 6, a VH CDR3 comprising the sequence shown in SEQ ID NO: 7, and/or the VL comprises a VL CDR1 comprising the sequence shown in SEQ ID NO: 10, a VL CDR2 comprising the sequence shown in SEQ ID NO: 11, a VL CDR3 comprising the sequence shown in SEQ ID NO: 12. In some aspects, the VH comprises the sequence shown in SEQ ID NO: 1, and/or the VL comprises the sequence shown in SEQ ID NO: 9. In some aspects, the first antibody comprises a heavy chain comprising the amino acid sequence shown in SEQ ID NO: 8, and/or a light chain comprising the amino acid sequence shown in SEQ ID NO: 13.

In some aspects, the second antibody variable domain specifically binds to BCMA. Information about BCMA is provided, for example, via UniProtKB ID #Q02223. In some aspects, the second antibody variable domain comprises three CDRs of a heavy chain variable region (VH) comprising the amino acid sequence shown in SEQ ID NO: 14, and/or three CDRs of a light chain variable region (VL) comprising the amino acid sequence shown in SEQ ID NO: 22. In some aspects, the VH comprises a VH CDR1 comprising the sequence shown in SEQ ID NO: 15, 16 or 17, a VH CDR2 comprising the sequence shown in SEQ ID NO: 18 or 19, a VH CDR3 comprising the sequence shown in SEQ ID NO: 20, and/or the VL comprises a VL CDR1 comprising the sequence shown in SEQ ID NO: 23, a VL CDR2 comprising the sequence shown in SEQ ID NO: 24, a VL CDR3 comprising the sequence shown in SEQ ID NO: 25. In some aspects, the VH comprises the sequence shown in SEQ ID NO: 14, and/or the VL comprises the sequence shown in SEQ ID NO: 22. In some aspects, the second antibody comprises a heavy chain comprising the amino acid sequence shown in SEQ ID NO: 21, and/or a light chain comprising the amino acid sequence shown in SEQ ID NO: 26.

In some aspects, the BCMA bispecific antibody is PF-06863135, also known as elranatamab. The BCMA bispecific antibody used in the Examples disclosed herein was PF-06863135, unless otherwise indicated. PF-06863135 is a heterodimeric humanized full-length bispecific antibody comprised of one B-cell maturation antigen (BCMA) binding arm and one cluster of differentiation (CD3) binding arm paired through hinge mutation technology. It utilizes a modified human IgG2Aa fragment crystallizable (Fc) region. PF-06863135 is described, for example in U.S. Pat. No. 9,969,809, which is hereby incorporated for all purposes. The sequences of PF-06863135 are shown in Table 19.

An effective amount of a BCMA-specific therapeutic agent may be administered according to the doses described herein.

### Anti-PD-1 and PD-L1 Antibody Therapeutic Agents

In some aspects, a therapeutic agent for use in the combination therapy of the present invention may be an anti-PD-1 or anti-PD-L1 antibody. The programmed death 1 (PD-1) receptor and PD-1 ligands 1 and 2 (PD-L1 and PD-L2, respectively) play integral roles in immune regulation. Expressed on activated T cells, PD-1 is activated by PD-L1 (also known as B7-H1) and PD-L2 expressed by stromal cells, tumor cells, or both, initiating T-cell death and localized immune suppression (Dong et al., Nat Med 1999; 5:1365-69; Freeman et al. J Exp Med 2000; 192:1027-34), potentially providing an immune-tolerant environment for tumor development and growth. Conversely, inhibition of this interaction can enhance local T-cell responses and mediate antitumor activity in nonclinical animal models (Iwai Y, et al. Proc Natl Acad Sci USA 2002; 99:12293-97).

Examples of anti-PD-1 and anti-PD-L1 antibodies that may useful in the combination therapy of the present invention include, but are not limited to, atezolizumab (TECENTRIQ®, MPDL3280A, Roche Holding AG), durvalumab (IMFINZI®, AstraZeneca PLC), nivolumab (OPDIVO®, ONO-4538, BMS-936558, MDX1106, Bristol-Myers Squibb Company), pembrolizumab (KEYTRUDA®, MK-3475, lambrolizumab, Merck & Co., Inc.), BCD-100 (BIOCAD Biopharmaceutical Company), tislelizumab (BGB-A317, BeiGene Ltd./Celgene Corporation), genolimzumab (CBT-501, CBT Pharmaceuticals), CBT-502 (CBT Pharmaceuticals), GLS-010 (Harbin Gloria Pharmaceuticals Co., Ltd.), sintilimab (161308, Innovent Biologics, Inc.), WBP3155 (CStone Pharmaceuticals Co., Ltd.), AMP-224 (GlaxoSmithKline plc), BI 754091 (Boehringer Ingelheim GmbH), BMS-936559 (Bristol-Myers Squibb Company), CA-170 (Aurigene Discovery Technologies), FAZ053 (Novartis AG), spartalizumab (PDR001, Novartis AG), LY3300054 (Eli Lilly & Company), MEDI0680 (AstraZeneca PLC), PDR001 (Novartis AG), sasanlimab (PF-06801591, Pfizer Inc.), cemiplimab (LIBTAYO®, REGN2810, Regeneron Pharmaceuticals, Inc.), camrelizumab (SHR-1210, Incyte Corporation), TSR-042 (Tesaro, Inc.), AGEN2034 (Agenus Inc.), CX-072 (CytomX Therapeutics, Inc.), JNJ-63723283 (Johnson & Johnson), MGD013 (MacroGenics, Inc.), AN-2005 (Adlai Nortye), ANA011 (AnaptysBio, Inc.), ANB011 (AnaptysBio, Inc.), AUNP-12 (Pierre Fabre Medicament S.A.), BBI-801 (Sumitomo Dainippon Pharma Co., Ltd.), BION-004 (Aduro Biotech), CA-327 (Aurigene Discovery Technologies), CK-301 (Fortress Biotech, Inc.), ENUM 244C8 (Enumeral Biomedical Holdings, Inc.), FPT155 (Five Prime Therapeutics, Inc.), FS118 (F-star Alpha Ltd.), hAb21 (Stainwei Biotech, Inc.), J43 (Transgene S.A.), JTX-4014 (Jounce Therapeutics, Inc.), KD033 (Kadmon Holdings, Inc.), KY-1003 (Kymab Ltd.), MCLA-134 (Merus B.V.), MCLA-145 (Merus B.V.), PRS-332 (Pieris AG), SHR-1316 (Atridia Pty Ltd.), STI-A1010 (Sorrento Therapeutics, Inc.), STI-A1014 (Sorrento Therapeutics, Inc.), STI-A1110 (Les Laboratoires Servier), and XmAb20717 (Xencor, Inc.).

In some aspects, a therapeutic agent in the combination therapy of the present invention is an anti-PD-1 antibody. In some aspects, the anti-PD-1 antibody may have any of the features or characteristics of any of the antibodies provided in WO2016/092419, which is hereby incorporated by reference for all purposes.

In some aspects, the anti-PD-1 antibody comprises three CDRs of a heavy chain variable region (VH) comprising the amino acid sequence shown in SEQ ID NO: 27, and/or three CDRs of a light chain variable region (VL) comprising the amino acid sequence shown in SEQ ID NO: 31. In some aspects, the VH comprises a VH CDR1 comprising the sequence shown in SEQ ID NO: 28, a VH CDR2 comprising the sequence shown in SEQ ID NO: 29, a VH CDR3 comprising the sequence shown in SEQ ID NO:30, and/or the VL comprises a VL CDR1 comprising the sequence shown in SEQ ID NO: 32, a VL CDR2 comprising the sequence shown in SEQ ID NO: 33, a VL CDR3 comprising the sequence shown in SEQ ID NO: 34. In some aspects, the VH comprises the sequence shown in SEQ ID NO: 27, and/or the VL comprises the sequence shown in SEQ ID NO: 31.

In some aspects, the anti-PD-1 antibody is sasanlimab (PF-06801591). Sasanlimab is a humanized, immunoglobulin G4 (IgG4) monoclonal antibody (mAb) that binds to the PD-1 receptor. By blocking its interaction with PD-L1 and PD-L2, PD-1 pathway-mediated inhibition of the immune response is released, leading to an anti-tumor immune response. Clinical anti-tumor activity with sasanlimab has been seen in a panel of anti-PD1 sensitive solid tumor types including non-small cell lung cancer and urothelial carcinoma. Sasanlimab is described, for example in U.S. Pat. No. 10,155,037, which is hereby incorporated for all purposes. The anti-PD-1 antibody used in the Examples disclosed herein was a therapeutic humanized anti-human PD-1 antibody (hIgG2a-D265A) prepared in-house, unless otherwise indicated.

An effective amount of an anti-PD-1 antibody or anti-PD-L1 antibody may be administered according to the doses described herein.

### Gamma Secretase Inhibitor Therapeutic Agents

In some aspects, a therapeutic agent for use in the combination therapy of the present invention may be a gamma secretase inhibitor (GSI). The terms “gamma secretase inhibitor”, “γ-secretase inhibitor” and “GSI” are used interchangeably herein to refer to refer to compounds (including pharmaceutically acceptable salts, solvates, and prodrugs thereof) or other agents that inhibit or reduce the biological activity of gamma secretase. Membrane-bound BCMA is actively cleaved by the protease activity of gamma secretase from a tumor cell surface and can undergo gamma secretase-mediated shedding. This may reduce target density on tumor cells for BCMA-specific therapeutic agents and release a soluble BCMA (sBCMA) fragment capable of interfering with BCMA-specific therapeutic agents. By inhibiting gamma secretase, membrane-bound BCMA may be preserved, increasing target density while reducing levels of sBCMA. Thus, administration of a GSI may enhance the activity of BCMA-specific therapeutic agents.

Examples of small molecule GSIs that may be useful in the combination therapy of the present invention include, but are not limited to, the dipeptide class of GSIs, the sulfonamide class of GSIs, the transition state mimic class of GSIs, the benzocaprolactam class of GSIs, and other GSIs known in the art. For example, the GSI may be selected from MK-0752 (Merck & Co., Inc.), MRK-003 (Merck & Co., Inc.), nirogacestat (PF-03084014, SpringWorks Therapeutics), R04929097 (Roche), semagacestat (LY450139, Eli Lilly & Company), BMS-906024 (Bristol-Myers Squibb Company) and DAPT, or the pharmaceutically acceptable salts thereof. Additional examples of GSIs include 1-(S)-endo-N-(1,3,3)-Trimethylbicyclo[2.2.1] hept-2-yl)-4-fluorophenyl sulfonamide, WPE-III-31 C, (S)-3-[N′-(3,5-difluorophenyl-alpha-hydroxyacetyl)-L-alaninyl]amino-2,3-dihydro-1-methyl-5-phenyl-1H-1,4-benzodiazepin-2-one, and (N)-[(S)-2-hydroxy-3-methyl-butyryl]-1-(L-alaninyl)-(S)-1-amino-3-methyl-4,5,6,7-tetrahydro-2H-3-benzazepin-2-one. See De Kloe & De Strooper (2014). Small Molecules That Inhibit Notch Signaling, In Bellen & Yamamoto (Eds.), Notch Signaling: Methods and Protocols, Methods in Mol. Biol., vol 1 187 (pp 311-322). New York, N.Y.: Springer-Science+Business Media.

In some aspects, a therapeutic agent in the combination therapy of the present invention is a GSI. In some aspects, the GSI may have any of the features or characteristics of any of the GSIs provided in WO2005/092864, which is hereby incorporated by reference for all purposes. In some aspects, the GSI is nirogacestat (PF-03084014, SpringWorks Therapeutics), or a pharmaceutically acceptable salt thereof. Nirogacestat is an oral, selective, small molecule GSI having the structure:

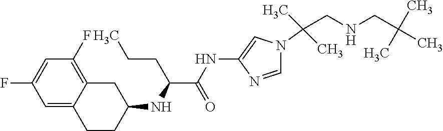 [SMILES: CCC[C@H](N[C@H]1CCc2cc(F)cc(F)c2C1)C(=O)Nc1cn(C(C)(C)CNCC(C)(C)C)cn1]

Nirogacestat is described, for example in U.S. Pat. Nos. 7,342,118, 7,795,447 and 7,951,958, which are hereby incorporated for all purposes. The GSI used in the Examples disclosed herein was nirogacestat, unless otherwise indicated.

An effective amount of a GSI may be administered according to the doses described herein. In some aspects, GSI is administered at a dose sufficient to upregulate surface expression of BCMA on tumor cells. In some aspects, GSI is administered at a dose sufficient to reduce shedding of BCMA on tumor cells. In some aspects, GSI is administered at a dose sufficient to reduce levels of sBCMA. In some aspects, GSI is administered at a dose sufficient to improve activity of BCMA-specific therapeutic agents.

### Therapeutic Agents

In some aspects, a therapeutic agent for use in the combination therapy of the present invention may comprise one or more of a biotherapeutic agent, a chemotherapeutic agent, an immunomodulating agent (e.g. thalidomide, lenalidomide, pomalidomide, iberdomide and apremilast), a proteasome inhibitor (e.g. bortezomib, carfilzomib and ixazomib), a corticosteroid (e.g. dexamethasone and prednisone), a histone deacetylase (HDAC) inhibitor (e.g. panobinostat), and a nuclear export inhibitor (e.g. selinexor). Further therapeutic agents for use in the combination therapy of the present invention include a cancer vaccine, immune cell therapy (e.g. CAR-T cell-based therapy), radiotherapy, a vaccine, a cytokine therapy (e.g., immunostimulatory cytokines including various signaling proteins that stimulate immune response, such as interferons, interleukins, and hematopoietic growth factors), a targeted cytokine, an inhibitor of other immunosuppressive pathways, an inhibitors of angiogenesis, a T cell activator, an inhibitor of a metabolic pathway, an mTOR (mechanistic target of rapamycin) inhibitor (e.g., rapamycin, rapamycin derivatives, sirolimus, temsirolimus, everolimus, and deforolimus), an inhibitor of an adenosine pathway, a gamma secretase inhibitor (e.g. nirogacestat), a tyrosine kinase inhibitor including but not limited to INLYTA®, ALK (anaplastic lymphoma kinase) inhibitors (e.g., crizotinib, ceritinib, alectinib, and sunitinib), a BRAF inhibitor (e.g., vemurafenib and dabrafenib), a PI3K inhibitor, a HPK1 inhibitor, an epigenetic modifier, an inhibitors or depletor of Treg cells and/or of myeloid-derived suppressor cells, a JAK (Janus Kinase) inhibitor (e.g., ruxolitinib and tofacitinb, varicitinib, filgotinib, gandotinib, lestaurtinib, momelotinib, pacritinib, and upadacitinib), a STAT (Signal Transducers and Activators of Transcription) inhibitor (e.g., STAT1, STAT3, and STAT5 inhibitors such as fludarabine), a cyclin-dependent kinase (CDK) or other cell cycle inhibitor, an immunogenic agent (for example, attenuated cancerous cells, tumor antigens, antigen presenting cells such as dendritic cells pulsed with tumor derived antigen or nucleic acids, a MEK inhibitor (e.g., trametinib, cobimetinib, binimetinib, and selumetinib), a GLS1 inhibitor, a PARP inhibitor (e.g. talazoparib, olaparib, rucaparib, niraparib), an oncolytic virus, gene therapies including DNA, RNA delivered directly or by adeno-associated viruses (AAV) or nanoparticles, an innate immune response modulator (e.g., TLRs, KIR, NKG2A), an IDO (Indoleamine-pyrrole 2,3-dioxygenase) inhibitor, a PRR (Pattern Recognition Receptors) agonist, and cells transfected with genes encoding immune stimulating cytokines such as but not limited to GM-CSF).

In some aspects, therapeutic agents for use in the combination therapy of the present invention may comprise an antibody, including but not limited to, an anti-CTLA-4 antibody, an anti-CD3 antibody, an anti-CD4 antibody, an anti-CD8 antibody, an anti-4-1BB antibody, an anti-PD-1 antibody, an anti-PD-L1 antibody, an anti-TIM3 antibody, an anti-LAG3 antibody, an anti-TIGIT antibody, an anti-OX40 antibody, an anti-IL-7Ralpha (CD127) antibody, an anti-IL-8 antibody, an anti-IL-15 antibody, an anti-HVEM antibody, an anti-BTLA antibody, an anti-CD38 antibody, an anti-CD40 antibody, an anti-CD40L antibody, anti-CD47 antibody, an anti-CSF1R antibody, an anti-CSF1 antibody, an anti-IL-7R antibody, an anti-MARCO antibody, an anti-CXCR4 antibodies, an anti-VEGF antibody, an anti-VEGFR1 antibody, an anti-VEGFR2 antibody, an anti-TNFR1 antibody, an anti-TNFR2 antibody, an anti-CD3 bispecific antibody, an anti-CD19 antibody, an anti-CD20, an anti-Her2 antibody, an anti-EGFR antibody, an anti-ICOS antibody, an anti-CD22 antibody, an anti-CD52 antibody, an anti-CCR4 antibody, an anti-CCR8 antibody, an anti-CD200R antibody, an anti-VISG4 antibody, an anti-CCR2 antibody, an anti-LILRb2 antibody, an anti-CXCR4 antibody, an anti-CD206 antibody, an anti-CD163 antibody, an anti-KLRG1 antibody, an anti-FLT3 antibody, an anti-B7-H4 antibody, an anti-B7-H3 antibody, an KLRG1 antibody, a BTN1A1 antibody, a BCMA antibody, an anti-SLAMF7 antibody, an anti-avb8 antibody, an anti-CD80 antibody or an anti-GITR antibody.

In some aspects, other examples of therapeutic agents for use in the combination therapy of the present invention may be directed or targeted to, 5T4; A33; alpha-folate receptor 1 (e.g. mirvetuximab soravtansine); Alk-1; BCMA (e.g. see WO2016166629 and others disclosed herein); BTN1A1 (e.g. see WO2018222689); CA19-9; CA-125 (e.g. abagovomab); Carboanhydrase IX; CCR2; CCR4 (e.g. mogamulizumab); CCR5 (e.g. leronlimab); CCR8; CD3 [e.g. blinatumomab (CD3/CD19 bispecific), PF-06671008 (CD3/P-cadherin bispecific), PF-06863135 (CD3/BCMA bispecific)]; CD19 (e.g. blinatumomab, MOR208); CD20 (e.g. ibritumomab tiuxetan, obinutuzumab, ofatumumab, rituximab, ublituximab); CD22 (inotuzumab ozogamicin, moxetumomab pasudotox); CD25; CD28; CD30 (e.g. brentuximab vedotin); CD33 (e.g. gemtuzumab ozogamicin); CD38 (e.g. daratumumab, daratumumab and hyaluronidase, and isatuximab), CD40; CD-40L; CD44v6; CD47 (e.g. Hu5F9-G4, CC-90002, SRF231, B6H12); CD52 (e.g. alemtuzumab); CD56; CD63; CD79 (e.g. polatuzumab vedotin); CD80; CD86; CD123; CD276/B7-H3 (e.g. omburtamab); CDH17; CEA; ClhCG; CTLA-4 (e.g. ipilimumab, tremelimumab), CXCR4; desmoglein 4; DLL3 (e.g. rovalpituzumab tesirine); DLL4; E-cadherin; EDA; EDB; EFNA4; EGFR (e.g. cetuximab, depatuxizumab mafodotin, necitumumab, panitumumab); EGFRvIII; Endosialin; EpCAM (e.g. oportuzumab monatox); FAP; Fetal Acetylcholine Receptor; FLT3 (e.g. see WO2018/220584); 4-1BB (CD137) [e.g. utomilumab/PF-05082566 (see WO2012/032433) or urelumab/BMS-663513], GD2 (e.g. dinutuximab, 3F8); GD3; GITR (e.g. TRX518); GloboH; GM1; GM2; HER2/neu [e.g. margetuximab, pertuzumab, trastuzumab; ado-trastuzumab emtansine, trastuzumab duocarmazine, PF-06804103 (see U.S. Pat. No. 8,828,401)]; HER3; HER4; ICOS; IL-10; ITG-AvB6; LAG-3 (e.g. relatlimab, IMP701); Lewis-Y; LG; Ly-6; M-CSF [e.g. PD-0360324 (see U.S. Pat. No. 7,326,414)]; (membrane-bound) IgE; MCSP; mesothelin; MIS Receptor type II; MUC1; MUC2; MUC3; MUC4; MUCSAC; MUCSB; MUC7; MUC16; Notch1; Notch3; Nectin-4 (e.g. enfortumab vedotin); OX40 [e.g. PF-04518600 (see U.S. Pat. No. 7,960,515)]; P-Cadherin [e.g. PF-06671008 (see WO2016/001810)]; PCDHB2; PD-1 [e.g. BCD-100, camrelizumab, cemiplimab, genolimzumab (CBT-501), MEDI0680, nivolumab, pembrolizumab, sasanlimab (PF-06801591, see WO2016/092419), sintilimab, spartalizumab, STI-A1110, tislelizumab, TSR-042, and others disclosed herein]; PD-L1 (e.g. atezolizumab, durvalumab, BMS-936559 (MDX-1105), LY3300054, and others disclosed herein); PDGFRA (e.g. olaratumab); Plasma Cell Antigen; PolySA; PSCA; PSMA; PTK7 [e.g. PF-06647020 (see U.S. Pat. No. 9,409,995)]; Ror1; SAS; SLAMF7 (e.g. elotuzumab); SHH; SIRPa (e.g. ED9, Effi-DEM); STEAP; sTn; TGF-beta; TIGIT; TIM-3; TMPRSS3; TNF-alpha precursor; TROP-2 (e.g., sacituzumab govitecan); TSPAN8; VEGF (e.g. bevacizumab, brolucizumab); VEGFR1 (e.g. ranibizumab); VEGFR2 (e.g. ramucirumab, ranibizumab); and Wue-1.

In some aspects, a therapeutic agent for use in the combination therapy of the present invention may be therapeutic antibody having any suitable format. For example, therapeutic antibodies may have any format as described elsewhere herein. In some aspects, a therapeutic antibody may be a naked antibody. In some aspects, a therapeutic antibody may be linked to a drug/agent (also known as an “antibody-drug conjugate” (ADC)). Drugs or agents that can be linked to an antibody in the ADC format can include, for example, cytotoxic agents, immunomodulating agents, imaging agents, therapeutic proteins, biopolymers, or oligonucleotides. Exemplary cytotoxic agents that may be incorporated in an ADC include an anthracycline, an auristatin, a dolastatin, a combretastatin, a duocarmycin, a pyrrolobenzodiazepine dimer, an indolino-benzodiazepine dimer, an enediyne, a geldanamycin, a maytansine, a puromycin, a taxane, a vinca alkaloid, a camptothecin, a tubulysin, a hemiasterlin, a spliceostatin, a pladienolide, and stereoisomers, isosteres, analogs, or derivatives thereof.

In some aspects, a therapeutic antibody against a particular antigen may incorporated into a multi-specific antibody (e.g. a bispecific or trispecific antibody). Bispecific antibodies are monoclonal antibodies that have binding specificity for at least two different antigens. In some aspects, a bispecific antibody comprises a first antibody variable domain and a second antibody variable domain, wherein the first antibody variable domain is capable of recruiting the activity of a human immune effector cell by specifically binding to an effector antigen located on the human immune effector cell, and wherein the second antibody variable domain is capable of specifically binding to a target antigen as provided herein. In some aspects, the antibody has an IgG1, IgG2, IgG3, or IgG4 isotype. In some aspects, the antibody comprises an immunologically inert Fc region. In some aspects the antibody is a human antibody or humanized antibody.

The human immune effector cell can be any of a variety of immune effector cells known in the art. For example, the immune effector cell can be a member of the human lymphoid cell lineage, including, but not limited to, a T cell (e.g., a cytotoxic T cell), a B cell, and a natural killer (NK) cell. The immune effector cell can also be, for example without limitation, a member of the human myeloid lineage, including, but not limited to, a monocyte, a neutrophilic granulocyte, and a dendritic cell. Such immune effector cells may have either a cytotoxic or an apoptotic effect on a target cell or other desired effect upon activation by binding of an effector antigen.

The effector antigen is an antigen (e.g., a protein or a polypeptide) that is expressed on the human immune effector cell. Examples of effector antigens that can be bound by the heterodimeric protein (e.g., a heterodimeric antibody or a bispecific antibody) include, but are not limited to, human CD3 (or CD3 (Cluster of Differentiation) complex), CD16, NKG2D, NKp46, CD2, CD28, CD25, CD64, and CD89. The target antigen is typically expressed on a target cell in a diseased condition (e.g. a cancer cell). Examples of target antigens for use in bispecific antibodies are disclosed herein.

In some aspects, a bispecific antibody provided herein binds to two different target antigens on the same target cell (e.g. two different antigens on the same tumor cell). Such antibodies may be advantageous, for example, for having increased specificity for a target cell of interest (e.g. for a tumor cell that expresses two particular tumor associated antigens of interest). For example, in some aspects, a bispecific antibody provided herein comprises a first antibody variable domain and a second antibody variable domain, wherein the first antibody variable domain is capable of specifically binding to a first target antigen as provided herein and the second antibody variable domain is capable of specifically binding to a second target antigen as provided herein.

In some aspects, a therapeutic agent for use in the combination therapy of the present invention may comprise immunomodulating agents, including thalidomide, lenalidomide, pomalidomide, iberdomide and apremilast, which may stimulate an immune response in a subject. Further immunomodulating agents include, pattern recognition receptor (PRR) agonists, immunostimulatory cytokines, immune cell therapy and cancer vaccines.

Pattern recognition receptors (PRRs) are receptors that are expressed by cells of the immune system and that recognize a variety of molecules associated with pathogens and/or cell damage or death. PRRs are involved in both the innate immune response and the adaptive immune response. PRR agonists may be used to stimulate the immune response in a subject. There are multiple classes of PRR molecules, including toll-like receptors (TLRs), RIG-I-like receptors (RLRs), nucleotide-binding oligomerization domain (NOD)-like receptors (NLRs), C-type lectin receptors (CLRs), and Stimulator of Interferon Genes (STING) protein.

Exemplary TLR agonists provided herein include agonists of TLR2, TLR3, TLR4, TLR5, TLR6, TLR7, TLR8, and TLR9. Examples of RLRs agonists that are useful in the treatment methods, medicaments, and uses of the present invention include, for example, short double-stranded RNA with uncapped 5′ triphosphate (RIG-I agonist); poly I:C (MDA-5 agonist), and BO-112 (MDA-A agonist). Examples of NLR agonists that are useful in the treatment methods, medicaments, and uses of the present invention include, for example, liposomal muramyl tripeptide/mifamurtide (NOD2 agonist). Examples of CLR agonists that are useful in the treatment methods, medicaments, and uses of the present invention include, for example, MD-fraction (a purified soluble beta-glucan extract from Grifola frondosa) and imprime PGG (a beta 1,3/1,6-glucan PAMP derived from yeast). Examples of STING agonists that are useful in the treatment methods, medicaments, and uses of the present invention include various immunostimulatory nucleic acids, such as synthetic double stranded DNA, cyclic di-GMP, cyclic-GMP-AMP (cGAMP), synthetic cyclic dinucleotides (CDN) such as MK-1454 and ADU-S100 (MIW815), and small molecules such as P0-424. Other PRRs include, for example, DNA-dependent Activator of IFN-regulatory factors (DAI) and Absent in Melanoma 2 (AIM2).

Immunostimulatory cytokines, include but not limited to, various signaling proteins that stimulate immune response, such as interferons, interleukins, and hematopoietic growth factors. In some aspects, exemplary immunostimulatory cytokines include, but are not limited to, GM-CSF, G-CSF, IFNγ, IFNα, IL-2 (e.g. denileukin difitox), IL-6, IL-7, IL-10, IL-11, IL-12, IL-15, IL-18, IL-21, and TNFα. Immunostimulatory cytokines may have any suitable format. In some aspects, an immunostimulatory cytokine may be a recombinant version of a wild-type cytokine. In some aspects, an immunostimulatory cytokine may be a mutein that has one or more amino acid changes as compared to the corresponding wild-type cytokine. In some aspects, an immunostimulatory cytokine may be incorporated into a chimeric protein containing the cytokine and at least one other functional protein (e.g. an antibody). In some aspects, an immunostimulatory cytokine may covalently linked to a drug/agent (e.g. any drug/agent as described elsewhere herein as a possible ADC component). In some aspects, the cytokines are pegylated.

Immune cell therapy involves treating a patient with immune cells that are capable of targeting cancer cells. Immune cell therapy includes, for example, tumor-infiltrating lymphocytes (TILs) and chimeric antigen receptor T cells (CAR-T cells).

Cancer vaccines include various compositions that contain tumor associated antigens (or which can be used to generate the tumor associated antigen in the subject) and thus can be used to provoke an immune response in a subject that will be directed to tumor cells that contain the tumor associated antigen. Example materials that may be included in a cancer vaccine include, attenuated cancerous cells, tumor antigens, antigen presenting cells such as dendritic cells pulsed with tumor derived antigen or nucleic acids encoding tumor associated antigens. In some aspects, a cancer vaccine may be prepared with a patient's own cancer cells. In some aspects, a cancer vaccine may be prepared with biological material that is not from a patient's own cancer cells. Cancer vaccines include, for example, sipuleucel-T and talimogene laherparepvec (T-VEC).

A combination therapy provided herein may comprise one or more chemotherapuetic agents. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI);

eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gammall and calicheamicin phill, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; antimetabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; FOLFOX including folinic acid, 5-FU and oxaliplatin; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as carboplatin; cisplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above.

In some aspects, a therapeutic agent for use in the combination therapy of the present invention may be an anti-hormonal agent that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole.

In some aspects, a therapeutic agent for use in the combination therapy of the present invention may be anti-androgens such as flutamide, nilutamide, bicalutamide, leuprolide, fluridil, apalutamide, enzalutamide, cimetidine and goserelin; KRAS inhibitors; MCT4 inhibitors; MAT2a inhibitors; tyrosine kinase/vascular endothelial growth factor (VEGF) receptor inhibitors such as sunitinib, axitinib, sorafenib, tivozanib; alk/c-Met/ROS inhibitors such as crizotinib, lorlatinib; mTOR inhibitors such as temsirolimus, gedatolisib; src/abl inhibitors such as bosutinib; cyclin-dependent kinase (CDK) inhibitors such as palbociclib, PF-06873600, abemaciclib and ribociclib; erb inhibitors such as dacomitinib; PARP inhibitors such as talazoparib, olaparib, rucaparib, niraparib; SMO inhibitors such as glasdegib, PF-5274857; EGFR T790M inhibitors such as PF-06747775; EZH2 inhibitors or other epigenetic modifer such as PF-06821497; PRMT5 such as PF-06939999 inhibitors; TGFRβr1 inhibitors such as PF-06952229; and pharmaceutically acceptable salts, acids or derivatives of any of the above.

### Treatment

Each therapeutic agent in a combination therapy of the invention may be administered either alone or in a medicament (also referred to herein as a pharmaceutical composition) which comprises the therapeutic agent and one or more pharmaceutically acceptable carriers, excipients and diluents, according to standard pharmaceutical practice.

Each therapeutic agent in a combination therapy of the invention may be administered simultaneously (i.e., in the same medicament), concurrently (i.e., in separate medicaments administered one right after the other in any order) or sequentially in any order. Sequential administration is particularly useful when the therapeutic agents in the combination therapy are in different dosage forms (one agent is a tablet or capsule and another agent is a sterile liquid) and/or are administered on different dosing schedules, e.g., a chemotherapeutic that is administered at least daily and a therapeutic that is administered less frequently, such as once weekly, once every two weeks, or once every three weeks.

In some aspects, a therapeutic agent in the combination therapy may be administered using the same dosage regimen (dose, frequency and duration of treatment) that is typically employed when the agent is used as monotherapy for treating the same cancer. In other aspects, the patient may receive a lower total amount of at least one of the therapeutic agents in the combination therapy than when the agent is used as monotherapy, e.g., smaller doses, less frequent doses, and/or shorter treatment duration.

Therapeutic agents in a combination therapy of the invention may be administered by any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, or topical administration. The therapeutic agents of the disclosure can be administered using any suitable method, such as by oral ingestion, nasogastric tube, gastrostomy tube, injection, infusion, implantable infusion pump, and osmotic pump. The suitable route and method of administration may vary depending on a number of factors such as the specific therapeutic agent being used, the rate of absorption desired, specific formulation or dosage form used, type or severity of the disorder being treated, the specific site of action, and conditions of the patient. Examples of parenteral routes of administration also include intraosseous and intrapleural.

Oral administration of a solid dose form of a therapeutic agent may be, for example, presented in discrete units, such as hard or soft capsules, pills, cachets, lozenges, or tablets, each containing a predetermined amount of at least one therapeutic agent. In another aspect, the oral administration may be in a powder or granule form. In another aspect, the oral dose form is sub-lingual, such as, for example, a lozenge. In such solid dosage forms, therapeutic agents are ordinarily combined with one or more adjuvants. Such capsules or tablets may contain a controlled-release formulation. In the case of capsules, tablets, and pills, the dosage forms also may comprise buffering agents or may be prepared with enteric coatings.

In another aspect, oral administration of a therapeutic agent may be in a liquid dose form. Liquid dosage forms for oral administration include, for example, pharmaceutically acceptable emulsions, solutions, suspensions, syrups, and elixirs containing inert diluents commonly used in the art (e.g., water). Such compositions also may comprise adjuvants, such as wetting, emulsifying, suspending, flavoring (e.g., sweetening), and/or perfuming agents.

In some aspects, therapeutic agents are administered in a parenteral dose form. “Parenteral administration” includes, for example, subcutaneous injections, intravenous injections, intraperitoneal injections, intramuscular injections, intrasternal injections, and infusion. Injectable preparations (i.e., sterile injectable aqueous or oleaginous suspensions) may be formulated according to the known art using suitable dispersing, wetting, and/or suspending agents, and include depot formulations.

In some aspects, therapeutic agents are administered in a topical dose form. “Topical administration” includes, for example, transdermal administration, such as via transdermal patches or iontophoresis devices, intraocular administration, or intranasal or inhalation administration. Compositions for topical administration also include, for example, topical gels, sprays, ointments, and creams. A topical formulation may include a compound that enhances absorption or penetration of the active ingredient through the skin or other affected areas. When therapeutic agents are administered by a transdermal device, administration will be accomplished using a patch either of the reservoir and porous membrane type or of a solid matrix variety. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, Finnin and Morgan, J. Pharm. Sci., 88 (10), 955-958 (1999).

Other carrier materials and modes of administration known in the pharmaceutical art may also be used with therapeutic agents. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman et al., Eds., Pharmaceutical Dosage Forms, Marcel Decker, New York, N.Y., 1980; and Kibbe et al., Eds., Handbook of Pharmaceutical Excipients (3.sup.rd Ed.), American Pharmaceutical Association, Washington, 1999.

Selecting a dosage regimen (also referred to herein as an administration regimen) for a combination therapy of the invention may depend on several factors, including the serum or tissue turnover rate of the entity, the level of symptoms, the immunogenicity of the entity, and the accessibility of the target cells, tissue or organ in the subject being treated. Preferably, a dosage regimen maximizes the amount of each therapeutic agent delivered to the patient consistent with an acceptable level of side effects. Accordingly, the dose amount and dosing frequency of each therapeutic agent or chemotherapeutic agent in the combination depends in part on the particular therapeutic agent, the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy.

In some aspects, therapeutic agents in a combination therapy of the invention may be administered to a subject at a dose of about 0.01 μg/kg, 0.02 μg/kg, 0.03 μg/kg, 0.04 μg/kg, 0.05 μg/kg, 0.06 μg/kg, 0.07 μg/kg, 0.08 μg/kg, 0.09 μg/kg, 0.1 μg/kg, 0.2 μg/kg, 0.3 μg/kg, 0.4 μg/kg, 0.5 μg/kg, 0.6 μg/kg, 0.7 μg/kg, 0.8 μg/kg, 0.9 μg/kg, 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 15 μg/kg, 20 μg/kg, 25 μg/kg, 30 μg/kg, 35 μg/kg, 40 μg/kg, 45 μg/kg, 50 μg/kg, 60 μg/kg, 70 μg/kg, 80 μg/kg, 90 μg/kg, 100 μg/kg, 110 μg/kg, 120 μg/kg, 130 μg/kg, 140 μg/kg, 150 μg/kg, 200 μg/kg, 250 μg/kg, 300 μg/kg, 400 μg/kg, 500 μg/kg, 600 μg/kg, 700 μg/kg, 800 μg/kg, 900 μg/kg, 1000 μg/kg, 1200 μg/kg, or 1400 μg/kg or higher.

In some aspects, therapeutic agents in a combination therapy of the invention may be administered to a subject at a dose from about 1 mg/kg to about 1000 mg/kg, from about 2 mg/kg to about 900 mg/kg, from about 3 mg/kg to about 800 mg/kg, from about 4 mg/kg to about 700 mg/kg, from about 5 mg/kg to about 600 mg/kg, from about 6 mg/kg to about 550 mg/kg, from about 7 mg/kg to about 500 mg/kg, from about 8 mg/kg to about 450 mg/kg, from about 9 mg/kg to about 400 mg/kg, from about 5 mg/kg to about 200 mg/kg, from about 2 mg/kg to about 150 mg/kg, from about 5 mg/kg to about 100 mg/kg, from about 10 mg/kg to about 100 mg/kg, or from about 10 mg/kg to about 60 mg/kg.

In some aspects, therapeutic agents in a combination therapy of the invention may be administered to a subject at a dose of at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 3.0 mg/kg, 5.0 mg/kg, 10 mg/kg, 25 mg/kg, 50 mg/kg body weight or more. See, e.g., Yang et al. (2003) New Engl. J. Med. 349:427-434; Herold et al. (2002) New Engl. J. Med. 346:1692-1698; Liu et al. (1999) J. Neurol. Neurosurg. Psych. 67:451-456; Portielji et al. (20003) Cancer Immunol. Immunother. 52:133-144.

In some aspects, a patient may be administered a fixed dose of a therapeutic agent of about or of at least about 0.05 μg, 0.2 μg, 0.5 μg, 1 μg, 10 μg, 100 μg, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 15 mg, 20 mg, 25 mg, 30 mg, 40 mg, 50 mg, 60 mg, 70 mg, 75 mg, 80 mg, 90 mg, 100 mg, 125 mg, 150 mg, 175 mg, 200 mg, 225 mg, 250 mg, 275 mg, 300 mg, 350 mg, 400 mg, 450 mg, 500 mg, 550 mg, 600 mg, 350 mg, 700 mg, 750 mg, 800 mg, 900 mg, 1000 mg or 1500 mg or higher. The fixed dose may be administered at intervals of, e.g. daily, every other day, three times per week, or one time each week, two weeks, three weeks, monthly, once every 2 months, once every 3 months, once every 4 months, etc.

For oral administration, therapeutic agents (e.g. typically small molecule chemotherapeutic agents) may be provided, in the form of tablets a dose of the therapeutic agent described herein.

In some aspects, therapeutic agents in a combination therapy of the invention may be administered at least once daily, once a day, twice a day, three times a day, four times a day, once every two days, once every three days, once a week, once every two weeks, once every three weeks, once every four weeks, once every 30 days, once every five weeks, once every six weeks, once a month, once every two months, once every three months, or once every four months in an oral, IV or SC dose.

The treatment methods described herein can continue for as long as the clinician overseeing the patient's care deems the treatment method to be effective. Non-limiting parameters that indicate the treatment method is effective include any one or more of the following: tumor shrinkage (in terms of weight and/or volume); a decrease in the number of individual tumor colonies; tumor elimination; and progression-free survival. Change in tumor size may be determined by any suitable method such as imaging. Various diagnostic imaging modalities well known in the art can be employed, such as computed tomography (CT scan), dual energy CDT, positron emission tomography, ultrasound, CAT scan and MRI. In some aspects, a combination therapy of the invention is used to treat a tumor that is large enough to be found by palpation or by imaging techniques well known in the art, such as MRI, ultrasound, or CAT scan.

Exemplary lengths of time associated with the course of therapy include about one week; about two weeks; about three weeks; about four weeks; about five weeks; about six weeks; about seven weeks; about eight weeks; about nine weeks; about ten weeks; about eleven weeks; about twelve weeks; about thirteen weeks; about fourteen weeks; about fifteen weeks; about sixteen weeks; about seventeen weeks; about eighteen weeks; about nineteen weeks; about twenty weeks; about twenty-one weeks; about twenty-two weeks; about twenty-three weeks; about twenty four weeks; about seven months; about eight months; about nine months; about ten months; about eleven months; about twelve months; about thirteen months; about fourteen months; about fifteen months; about sixteen months; about seventeen months; about eighteen months; about nineteen months; about twenty months; about twenty one months; about twenty-two months; about twenty-three months; about twenty-four months; about thirty months; about three years; about four years and about five years.

The presently described combinations and methods can be used to treat a patient suffering from any condition that can be remedied or prevented by the methods provided herein, such as cancer and/or cancer-associated disease.

In some aspects, the condition is a cancer, including but not limited to, carcinoma, lymphoma, leukemia, myeloma, blastoma, and sarcoma. In some aspects, the cancer may include cancer-associated diseases, including a B-cell related cancer and/or cancer-associated disease including but are not limited to, multiple myeloma, malignant plasma cell neoplasm, lymphoma, Hodgkin's lymphoma, nodular lymphocyte predominant Hodgkin's lymphoma, Kahler's disease and Myelomatosis, plasma cell leukemia, plasmacytoma, monoclonal gammopathy of unknown significance (MGUS), smoldering myeloma, light chain amyloidosis, osteosclerotic myeloma, B-cell prolymphocytic leukemia, hairy cell leukemia, B-cell non-Hodgkin's lymphoma (NHL), acute myeloid leukemia (AML), chronic lymphocytic leukemia (CLL), acute lymphocytic leukemia (ALL), chronic myeloid leukemia (CML), follicular lymphoma, Burkitt's lymphoma, marginal zone lymphoma, mantle cell lymphoma, large cell lymphoma, precursor B-lymphoblastic lymphoma, myeloid leukemia, Waldenstrom's macroglobulienemia, diffuse large B cell lymphoma, mucosa-associated lymphatic tissue lymphoma, small cell lymphocytic lymphoma, primary mediastinal (thymic) large B-cell lymphoma, lymphoplasmactyic lymphoma, marginal zone B cell lymphoma, splenic marginal zone lymphoma, intravascular large B-cell lymphoma, primary effusion lymphoma, lymphomatoid granulomatosis, T cell/histiocyte-rich large B-cell lymphoma, primary central nervous system lymphoma, primary cutaneous diffuse large B-cell lymphoma (leg type), EBV positive diffuse large B-cell lymphoma of the elderly, diffuse large B-cell lymphoma associated with inflammation, ALK-positive large B-cell lymphoma, plasmablastic lymphoma, large B-cell lymphoma arising in HHV8-associated multicentric Castleman disease, B-cell lymphoma unclassified with features intermediate between diffuse large B-cell lymphoma and Burkitt lymphoma, B-cell lymphoma unclassified with features intermediate between diffuse large B-cell lymphoma and classical Hodgkin lymphoma, and other B-cell related lymphoma.

In some aspects, the cancer is gastric cancer, small intestine cancer, head and neck cancer (e.g., squamous cell head and neck cancer), thymic cancer, epithelial cancer, salivary cancer, liver cancer, biliary cancer, neuroendocrine tumors, stomach cancer, thyroid cancer, lung cancer (e.g., non-small-cell lung cancer, small cell lung cancer), mesothelioma, ovarian cancer, breast cancer, prostate cancer, kidney cancer, esophageal cancer, pancreatic cancer, glioma, renal cancer (e.g., renal cell carcinoma), bladder cancer, cervical cancer, uterine cancer, vulvar cancer, endometrial cancer, penile cancer, testicular cancer, anal cancer, choriocarcinoma, colon cancer, colorectal cancer, oral cancer, skin cancer, Merkel cell carcinoma, glioblastoma, brain tumor, bone cancer, eye cancer, melanoma, or cancer with high microsatellite instability (MSI-H).

A combination therapy of the invention may be used prior to or following surgery to remove a tumor and may be used prior to, during or after radiation therapy.

In some aspects, a combination therapy of the invention is administered to a patient who has not been previously treated with a therapeutic or chemotherapeutic agent, i.e., is treatment-naïve. In other aspects, the combination therapy is administered to a patient who failed to achieve a sustained response after prior therapy with a therapeutic or chemotherapeutic agent, i.e., is treatment-experienced. In some aspects, the subject has received a prior therapy to treat the tumor and the tumor is relapsed or refractory.

Encompassed by the invention provided herein are combination therapies that have additive potency or an additive therapeutic effect while reducing or avoiding unwanted or adverse effects. The invention also encompasses synergistic combinations where the therapeutic efficacy is greater than additive, while unwanted or adverse effects are reduced or avoided. In certain aspects, the methods and compositions provided herein permit treatment or prevention of diseases and disorders wherein treatment is improved by an enhanced anti-tumor response using lower and/or less frequent doses of at least therapeutic agent in a combination therapy to at least one of: i) reduce the incidence of unwanted or adverse effects caused by the administration of the therapeutic agents separately, while at least maintaining efficacy of treatment; ii) increase patient compliance, and iii) improve efficacy of the anti-tumor treatment.

### Kits

The therapeutic agents of the combination therapies of the present invention may conveniently be combined in the form of a kit suitable for co-administration of the compositions.

In one aspect, a kit comprises at least a first container and a second container and a package insert. The first container contains at least one dose of a first therapeutic agent, and the second container contains at least one dose of a second therapeutic agent of the combination therapy. The package insert/label comprises instructions for treating a patient for cancer and/or cancer-associated disease using the therapeutic agents. The first and second containers may be comprised of the same or different shape (e.g., vials, syringes and bottles) and/or material (e.g., plastic or glass). The kit may further comprise other materials that may be useful in administering the therapeutic agents, such as diluents, filters, IV bags and lines, needles and syringes.

### Clinical Studies

A Phase 1, open label, multi dose, multi center, dose escalation, safety, pharmacokinetic (PK) and pharmacodynamic study of PF-06863135 in adult patients with advanced multiple myeloma who have relapsed from or are refractory to standard therapy is ongoing (NCT03269136). This is a two part study to assess the safety and tolerability of increasing dose levels of PF-06863135 in Part 1, and establish the recommended Phase 2 dose (RP2D) in Part 2. This phase 1 study is described in Example 10. Two additional clinical studies of PF06863135 (elranatamab) monotherapy are described in Examples 11 and 12.

Further clinical evaluation of PF-06863135 in combination with any of therapeutics agents disclosed herein may be conducted: PF-06863135 in combination with an anti-PD-1/PD-L1 antibody (e.g. sasanlimab/PF-06801591), PF-06863135 in combination with an immunomodulating agent (e.g. thalidomide, lenalidomide, pomalidomide, iberdomide and apremilast), PF-06863135 in combination with a gamma secretase inhibitor (e.g. nirogacestat), PF-06863135 in combination with other treatments such as a biotherapeutic agent (e.g. CD38 antibodies daratumumab, daratumumab and hyaluronidase, and isatuximab, and SLAMF7 antibody elotuzumab), a chemotherapeutic agent (e.g. melphalan, vincristine, cyclophosphamide, etoposide, doxorubicin, liposomal doxorubicin, and dendamustine), a proteasome inhibitor (e.g. bortezomib, carfilzomib and ixazomib), a corticosteroid (e.g. dexamethasone and prednisone), a histone deacetylase (HDAC) inhibitor (e.g. panobinostat), and a nuclear export inhibitor (e.g. selinexor). Examples 12 to 16 describe a few planned combination therapy clinical studies of PF06863135 (elranatamab).

### General Methods

Standard methods in molecular biology are described Sambrook, Fritsch and Maniatis (1982 & 1989 2nd Edition, 2001 3rd Edition) Molecular Cloning, A Laboratory Manual, Cold Spring Harbor Laboratory Press, Cold Spring Harbor, N.Y.; Sambrook and Russell (2001) Molecular Cloning, 3rd ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, N.Y.; Kay et al. (1996) Phage Display of Peptides and Proteins: A Laboratory Manual, Academic Press, San Diego, Calif.; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, N.J.; Givan (2001) Flow Cytometry, 2nd ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, Oreg.; Sigma-Aldrich (2003) Catalogue, St. Louis, Mo.).

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne, et al. (2000)

Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

### Examples

### Example 1: In Vitro Study of PD-1 Induction on CD8+ T Cells Co-Cultured with MM.1S Multiple Myeloma Cells Treated with BCMAxCD3 Bispecific Antibody

This example illustrates that treatment with BCMAxCD3 bispecific induces PD-1 expression on CD8⁺ T cells.

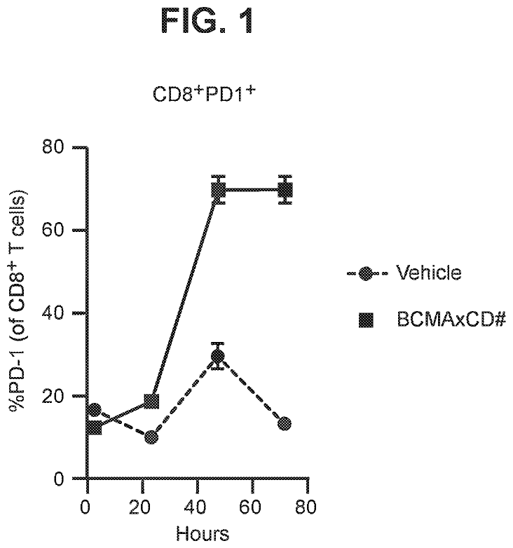

CD3+ T cells from PBMCs (Stem Cell Technologies) were negatively selected using EasySep Human T cell enrichment kit (Stem Cell Technologies). 10,000 target Multiple Myeloma MM.1S cells expressing luciferase (MM.1S-luc) were seeded with 50,000 CD3⁺ pan T cells in a clear 96-well V-bottom plate. Cells were treated with 1 nM BCMAxCD3 bispecific antibody and PD-1 expression was analyzed at 3, 24, 48 and 72 hours after addition of the BCMAxCD3 bispecific. At the specified time points, cells were collected from the wells, washed with PBS+2% FBS, and stained with ZombieNIR Viability dye (Biolegend) in PBS for 20 minutes at room temperature, followed by staining with antibodies against human CD8 and PD-1 (Biolegend). Samples were analyzed using FlowJo flow cytometry analysis software. Dead cells were removed from analysis by gating on ZombieNIR negative population. Samples were further gated on CD8⁺ positive population. Percentage of PD-1+ cells is expressed as PD-1 positive cells within the CD8+ population. The results summarized in FIG. 1 and Table 1 demonstrate that treatment of BCMA-expressing MM.15 multiple myeloma cells with the BCMAxCD3 bispecific antibody induces PD-1 expression on CD8+ T cells.

TABLE 1
% PD-1 cells after treatment with BCMA × CD3 bispecific

| | % PD-1 (of CD8+ T cells) | |
|---|---|---|
| | [±Standard Deviation] | |
| Hours | Vehicle | BCMA × CD3 Bispecific |
| 3 | 17.1 ± 0.6 | 12.8 ± 0.6 |
| 24 | 10.3 ± 0.4 | 18.9 ± 1.2 |
| 48 | 29.8 ± 3.0 | 69.9 ± 3.1 |
| 72 | 13.6 ± 0.5 | 69.9 ± 3.1 |

### Example 2: In Vivo Study of BCMAxCD3 Bispecific Antibody in Combination with Anti-PD-1 Antibody in MM.1S-PDL1 Orthotopic and Subcutaneous Mouse Model

This example illustrates combination efficacy of BCMAxCD3 bispecific with anti-PD-1 antibody in (A.) orthotopic MM.1S-Luc-PD-L1 and (B.) MM.1S-PD-L1 multiple myeloma models as compared to BCMAxCD3 bispecific or anti-PD-1 antibody alone.

### A. Orthotopic Mouse Model

MM.1 S-Luc multiple myeloma cells were engineered to express PD-L1 and are referred to as MM.1S-Luc-PD-L1. MM.1S-Luc-PD-L1 cells were prepared as a single cell suspension of 5×10⁶ cells for intravenous (IV) inoculation into NSG mice.

Tumor growth was monitored by luminescent imaging by intraperitoneal (IP) injection of luciferin solution in DPBS and imaged using Perkin Elmer IVIS Spectrum camera system. Animals were administered 2×10⁷ expanded human T cells IV 19 days after tumor cell inoculation. Two days following T cell administration, a single dose of the BCMAxCD3 bispecific (10 μg/kg) was administered as a bolus IV injection. Anti-PD-1 antibody was administered twice a week as a bolus IP injection at 5 mg/kg for a total of 6 injections.

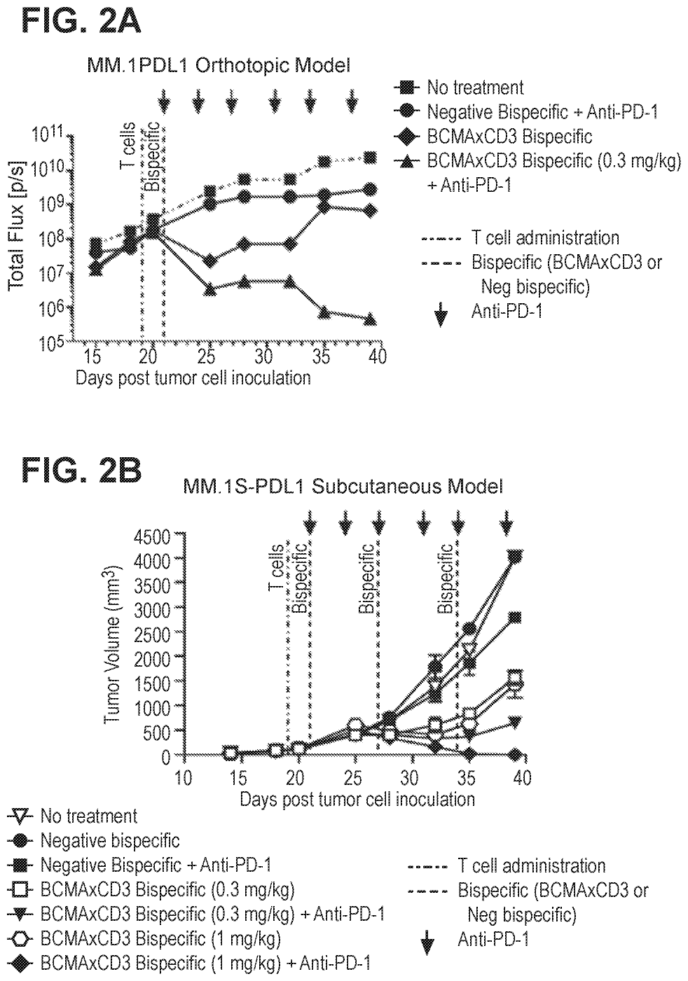

Tumor growth was monitored via imaging measurements collected twice weekly. Mice were imaged using the Perkin Elmer IVIS Spectrum camera system with automatic parameter determination and a maximum imaging time of 3 minutes. Living Image software was used to collect the data. Regions of Interest (ROIs) were drawn around the entire body of the mouse, excluding as much as possible of the tail. Background flux, as measured on the anesthesia manifold, is subtracted from each ROI. Tumor measurements are expressed as total flux expressed in photons/sec (p/s). Study was terminated at day 40 post tumor inoculation. The results summarized in FIG. 2A and Table 2 demonstrate that treatment with BCMAxCD3 bispecific and anti-PD-1 antibody is more efficacious compared to treatment with bispecific or antibody treatment alone.

TABLE 2
Tumor measurements e×pressed as total flux p/s after treatment

| Days | | | | |
|---|---|---|---|---|
| post | | | | |
| tumor | Negative | BCMAxCD3 | | |
| cell | bispecific + | bispecific | BCMAxCD3 | |
| inocu- | anti-PD-1 | 10 μg/kg + anti- | bispecific | No |
| lation | 5 mg/kg | PD-1 5 mg/kg | 10 μg/kg | treatment |
| 15 | 4.01 × 10⁷ | 1.31133334 × 10⁷ | 1.51333334 × 10⁷ | 7.2 × 10⁷ |
| | ± | ± | ± | ± |
| | 2.25 × 10⁷ | 3.1997153 × 10⁸ | 2.325463 × 10⁶ | 5 × 10⁷ |
| 18 | 5.535 × 10⁷ | 6.54333334 × 10⁷ | 8.34333334 × 10⁷ | 1.68 × 10⁸ |
| | ± | ± | ± | ± |
| | 1.735 × 10⁷ | 1.36827548 × 10⁷ | 2.40911003 × 10⁷ | 1.54 × 10⁸ |
| 20 | 1.855 × 10⁸ | 1.586666667 × | 1.788 × 10⁸ | 3.76 × 10⁸ |
| | ± | 10⁸ ± | ± | ± |
| | 4.75 × 10⁷ | 1.92382721 × 10⁷ | 6.89374596 × 10⁷ | 1.31 × 10⁸ |
| 25 | 1.052 × 10⁹ | 3.4466667 × 10⁶ | 2.2729 × 10⁷ | 2.455 × 10⁹ |
| | ± | ± | ± | ± |
| | 2.08 × 10⁸ | 2.8719195 × 10⁶ | 1.99389279 × 10⁷ | 9.55 × 10⁸ |
| 28 | 1.73 × 10⁹ | 5.792 × 10⁸ | 7.08166667 × 10⁷ | 5.555 × 10⁹ |
| | ± | ± | ± | ± |
| | 1.3 × 10⁸ | 4.8055591 × 10⁸ | 6.7101553 × 10⁷ | 2.455 × 10⁹ |
| 32 | 1.73 × 10⁹ | 5.792 × 10⁸ | 7.07566667 × 10⁷ | 5.545 × 10⁹ |
| | ± | ± | ± | ± |
| | 1.3 × 10⁸ | 4.8055591 × 10⁸ | 6.71310595 × 10⁷ | 2.455 × 10⁹ |
| 35 | 1.93 × 10⁹ | 7.373334 × 10⁵ | 8.712333334 × | 1.8295 × 10¹⁰ |
| | ± | ± | 10⁸ ± | ± |
| | 7.8 × 10⁸ | 4.980711 × 10⁵ | 8.444262754 × 10⁸ | 1.3305 × 10¹⁰ |
| 39 | 2.825 × 10⁹ | 4.654667 × 10⁵ | 6.775666667 × | 2.446 × 10¹⁰ |
| | ± | ± | 10⁸ ± | ± |
| | 1.745 × 10⁹ | 3.153991 × 10⁵ | 6.363327336 × 10⁸ | 1.454 × 10¹⁰ |

### B. Subcutaneous Mouse Model

MM.1S multiple myeloma cells were engineered to express PD-L1 and are referred to as MM.1S-PD-L1. Pre-activated and expanded T cells (20×10⁶) were administered on Day 19 after MM.1S-PDL1 tumor cell inoculation subcutaneously (SC). BCMAxCD3 bispecific (0.3 or 1 mg/kg) or negative bispecific (1 mg/kg) was administered IV on Day 21 and dosed Q7Dx3. Anti-PD-1 mAb was administered at 5 mg/kg intraperitoneally (IP) twice a week starting on Day 21. Tumor measurements were recorded 2 to 3 times a week using digital calipers. N (at study start) is 5-12 animals per group. The results summarized in FIG. 2B and Table 3 demonstrate that treatment with BCMAxCD3 bispecific and anti-PD-1 antibody is more efficacious compared to treatment with bispecific or antibody treatment alone.

TABLE 3
Tumor measurements after treatment (Tumor Volume ± SEM (mm³)).

| | | | Negative | BCMA × CD3 bispecific | | BCMA × CD3 bispecific | |
|---|---|---|---|---|---|---|---|
| Days post | | | bispecific + | 0.3 mg/kg | | 1 mg/kg | |
| tumor cell | No | Negative | anti-PD-1 | | +anti-PD-1 | | +anti-PD-1 |
| inoculation | treatment | bispecific | 5 mg/kg | Alone | 5 mg/kg | Alone | 5 mg/kg |
| 14 | 39.6 ± 6.9 | 34.1 ± 5.3 | 30.7 ± 4.6 | 34.4 ± 9.5 | 33.6 ± 5.3 | 26.4 ± 7.5 | 33.6 ± 5.3 |
| 18 | 80.3 ± 16.9 | 71.5 ± 13.7 | 64.5 ± 10 | 91.6 ± 18 | 74.5 ± 12.2 | 92 ± 17.1 | 74.5 ± 12.2 |
| 20 | 108.7 ± 27 | 121.4 ± 27.3 | 114.7 ± 21 | 122.5 ± 20.7 | 110.5 ± 18.6 | 130.7 ± 24.6 | 110.5 ± 18.6 |
| 25 | 407.8 ± 87.4 | 401.7 ± 30.5 | 401.3 ± 42.9 | 403 ± 59 | 504.4 ± 67.4 | 601.1 ± 70 | 504.4 ± 67.4 |
| 28 | 699.4 ± 126.2 | 761.6 ± 97.1 | 694.7 ± 69.5 | 403.3 ± 67.6 | 341 ± 86.9 | 482.9 ± 32.6 | 341 ± 86.9 |
| 32 | 1379.8 ± 308.6 | 1789.8 ± 233.4 | 1238.6 ± 146.2 | 603.6 ± 151.6 | 164.9 ± 108.9 | 410.5 ± 82.7 | 164.9 ± 108.9 |
| 35 | 2125.2 ± 61.6 | 2562.5 ± 81.8 | 1861.3 ± 238.4 | 835.1 ± 77.2 | 18.5 ± 9.9 | 627.9 ± 111.8 | 18.5 ± 9.9 |
| 39 | 4017.3 ± 103 | 4012.4 ± 106.2 | 2792.6 ± 4.7 | 1569.3 ± 151.8 | 2.9 ± 2.9 | 1436.8 ± 276.4 | 2.9 ± 2.9 |

### Example 3: In Vitro Study to Detect Cell Surface BCMA Expression in Multiple Myeloma Cell Lines Treated with Gamma Secretase Inhibitors (GSI)

This example illustrates upregulation of cell surface BCMA expression in multiple myeloma cell lines treated with GSI.

Multiple myeloma cells (MM.1S, OPM2, H929, Molp8, RPMI8226) were seeded in a 96-well U bottom plate at 40,000 cells/well. Cells were incubated for 24 hours in the presence of GSI diluted in RPMI (0.1% DMSO). The following concentrations of the GSI were tested: 1000 nM, 500 nM, 100 nM, 50 nM, 25 nM, 10 nM, 5 nM, 2.5 nM, 1 nM, 0.1 nM, 0.01 nM. 24 hours later cells were collected and washed with PBS+2% FBS, followed by staining with ZombieNIR Viability Dye (Biolegend) at 1 in 500 dilution in PBS at room temperature for 20 minutes. Next, cells were washed with PBS+2% FBS and stained with anti-BCMA PE-labelled antibody (Biolegend) diluted in PBS+2% FBS for 30 minutes at 4° C. Cells were acquired on BD Flow Cytometer and analyzed using FlowJo flow cytometry analysis software. Dead cells were removed from analysis by gating on ZombieNIR negative population. BCMA mean fluorescence intensity (MFI) was plotted against GSI concentration to establish EC50.

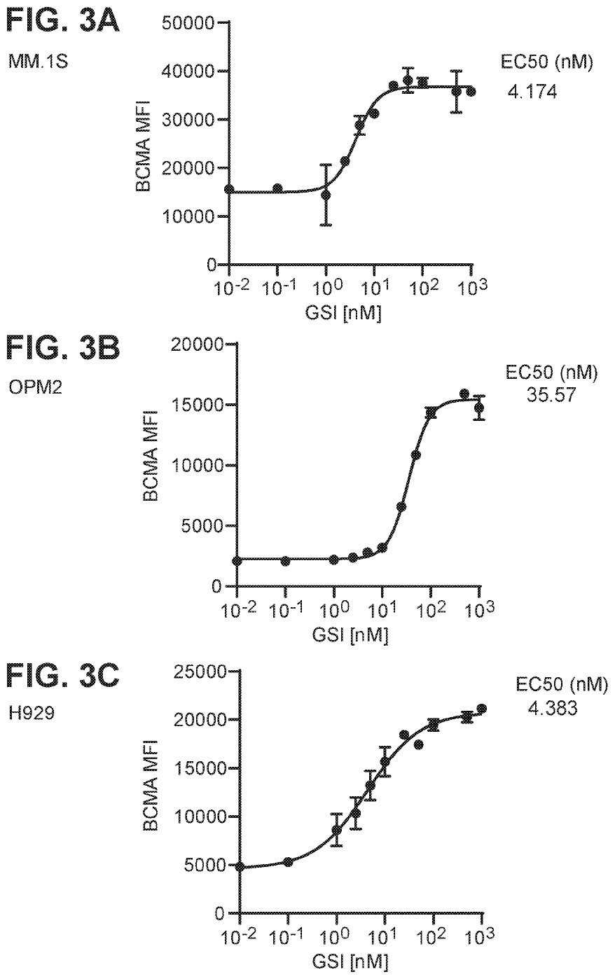

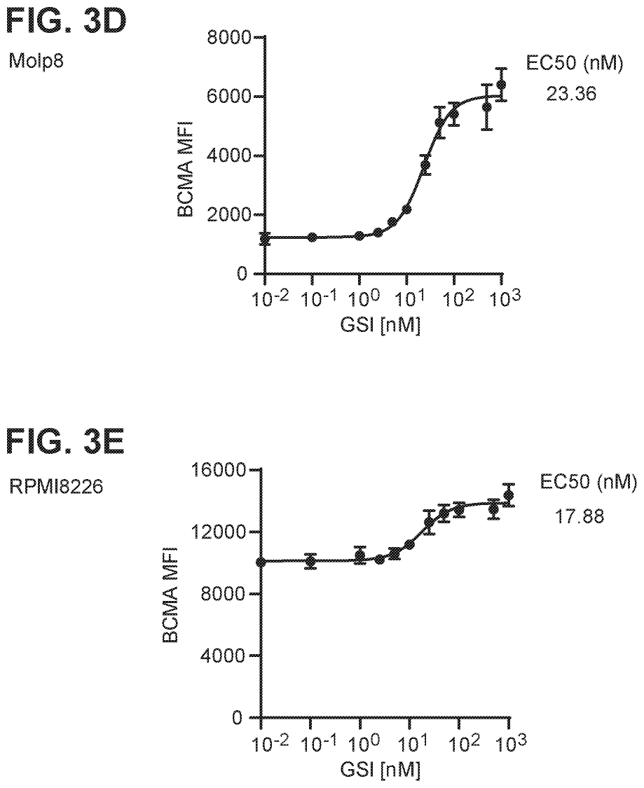

The results summarized in FIG. 3A-3E and Table 4 show that GSI treatment upregulates BCMA expression on the cell surface of multiple myeloma cell lines MM.1S, OPM2, H929, Molp8, and RPMI8226, respectively.

TABLE 4
Mean Florescence Intensity ± Standard Deviation

| GSI | BCMA (-PE) MFI | | | | |
|---|---|---|---|---|---|
| [nM] | MM.1S | OPM2 | H929 | Molp8 | RPMI8226 |
| 1000 | 35780 ± | 14747.5 ± | 21156 ± | 6401 ± | 14362 ± |
| | 21.2 | 965.2 | 338 | 550.1 | 705.7 |
| 500 | 35780 ± | 15910.5 ± | 20285.5 ± | 5640.5 ± | 13464.5 ± |
| | 4279.4 | 307.6 | 542.4 | 758.7 | 624.4 |
| 100 | 37582.5 ± | 14364 ± | 19455.5 ± | 5412 ± | 13416 ± |
| | 1033.1 | 403.1 | 563.6 | 383.3 | 452.5 |
| 50 | 38111 ± | 10863 ± | 17424.5 ± | 5120 ± | 13190.5 ± |
| | 2506 | 19.8 | 48.8 | 533.2 | 555.1 |
| 25 | 37026.5 ± | 6575.5 ± | 18445.5 ± | 3691 ± | 12617 ± |
| | 370 | 115.3 | 309 | 323.9 | 762.3 |
| 10 | 31248 ± | 3190.5 ± | 15670 ± | 2183.5 ± | 11181.5 ± |
| | 335.2 | 88.4 | 1516 | 81.3 | 193 |
| 5 | 28832 ± | 2788.5 ± | 13226 ± | 1761.5 ± | 10589.5 ± |
| | 1926.2 | 177.5 | 1531.6 | 74.2 | 351.4 |
| 2.5 | 21412.5 ± | 2358.5 ± | 10344.5 ± | 1401 ± | 10226.5 ± |
| | 614.5 | 136.5 | 1617.2 | 107.5 | 277.9 |
| 1 | 14396 ± | 2185 ± | 8615.5 ± | 1288.5 ± | 10483.5 ± |
| | 6222.5 | 15.6 | 1656.8 | 16.3 | 536.7 |
| 0.1 | 15775.5 ± | 2079.5 ± | 5305.5 ± | 1242.5 ± | 10099 ± |
| | 316.1 | 82.7 | 290.6 | 137.9 | 455.4 |
| 0.01 | 15589.5 ± | 2093.5 ± | 4824.5 ± | 1191.5 ± | 10028 ± |
| | 230 | 71.4 | 183.1 | 188.8 | 29.7 |
| 0 | 15362 | 2069.5 ± | 4908 ± | 1231 ± | 10142 ± |
| | | 23.3 | 138.6 | 79.2 | 448.3 |

### Example 4: In Vitro Study to Detect Cell Surface BCMA Expression in a Time-Dependent Manner in Multiple Myeloma Cell Lines Treated with GSI

This example illustrates that treating multiple myeloma cell lines with GSI increases BCMA cell surface expression in a time-dependent manner and that BCMA surface levels return to baseline after GSI is removed from the cultures.

Multiple myeloma cells (MM.1S, OPM2, H929, Molp8, RPMI8226) were seeded in a 6-well plate at 800,000 cells/2 ml/well with GSI diluted at 1 μM in RPMI medium (with 0.1% DMSO). Cells were collected to evaluate cell surface BCMA expression at baseline, and then at 3 hours, 6 hours and 24 hours after addition of the GSI. After 24 hours of incubation with the GSI, cells were washed twice in PBS and re-plated in fresh 6-well plates. Cells were further collected for staining at 3 hours, 6 hours and 24 hours after the GSI was washed out. At the indicated time points, samples were stained with a ZombieNIR Viability dye (Biolegend) for 20 minutes at room temperature at 1 in 500 dilution in PBS, washed with PBS+2% FBS and further stained with anti-BCMA PE-labelled antibody diluted in PBS+2% FBS for 30 minutes at 4° C. Samples were acquired on BD Flow Cytometer and analyzed using FlowJo software. Dead cells were removed from the analysis by gating on ZombieNIR negative population. BCMA MFI was plotted as a histogram.

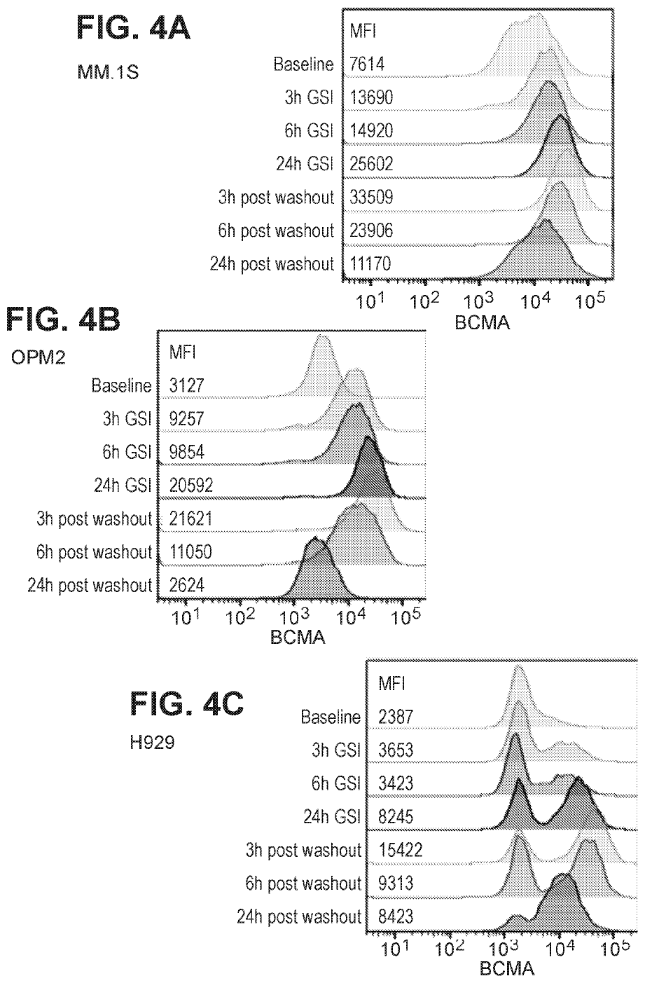

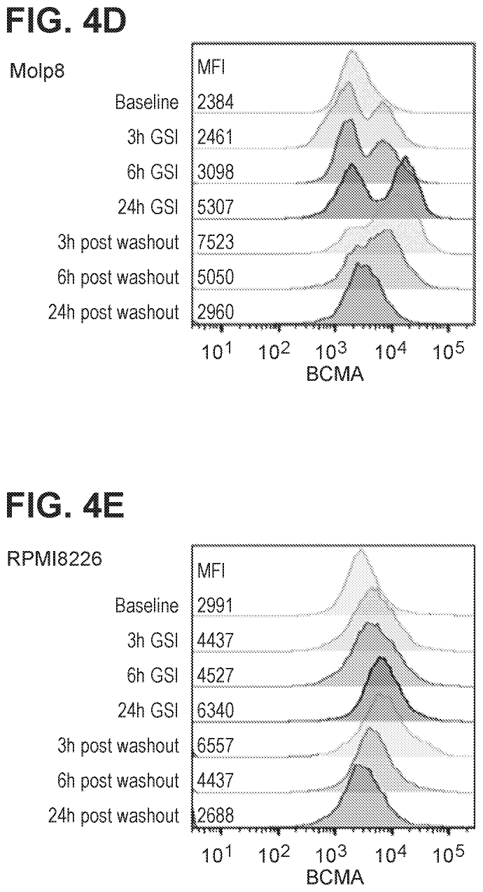

The results summarized in FIG. 4A-4E and Table 5 show that GSI upregulates cell surface BCMA expression on MM.1S, OPM2, H929, Molp8, and RPMI8226 cells, respectively, in a time-dependent manner and that upregulated surface BCMA expression does not persist after removing GSI from the cultures.

TABLE 5
Mean Florescence Intensity ± Standard Deviation

| Time | BCMA (-PE) MFI | | | | |
|---|---|---|---|---|---|
| Point | MM.1S | OPM2 | H929 | Molp8 | RPMI8226 |
| Baseline | 7614 | 3127 | 2387 | 2384 | 2991 |
| 3 h after GSI | 13690 | 9257 | 3653 | 2461 | 4437 |
| 6 h after GSI | 14920 | 9854 | 3423 | 3098 | 4527 |
| 24 h after GSI | 25602 | 20592 | 8245 | 5307 | 6340 |
| 3 h post wash | 33509 | 21621 | 15422 | 7523 | 6557 |
| 5 h post wash | 23906 | 11050 | 9313 | 5050 | 4437 |
| 24 h post wash | 11170 | 2624 | 8423 | 2960 | 2688 |

### Example 5: In Vitro Study to Detect Soluble BCMA (sBCMA) Levels in Multiple Myeloma Cell Lines Treated with GSI

This example illustrates reduced shedding of sBCMA in multiple myeloma cell lines treated with GSI.

Multiple myeloma cells (MM.1S, OPM2, H929, Molp8, RPMI8226) were seeded in a 96-well U-bottom plate at 40,000 cells/well. Cells were incubated for 24 hours in the presence of GSI diluted in RPMI medium (0.1% DMSO). The following concentrations of the GSI were tested: 1000 nM, 500 nM, 100 nM, 50 nM, 25 nM, 10 nM, 5 nM, 2.5 nM, 1 nM, 0.1 nM, 0.01 nM. After 24 hours, cell culture medium was collected and concentration of sBCMA was measured in supernatants using Human BCMA/TNFRSF17 DuoSet ELISA kit (R&D Systems) according to manufacturer's instructions.

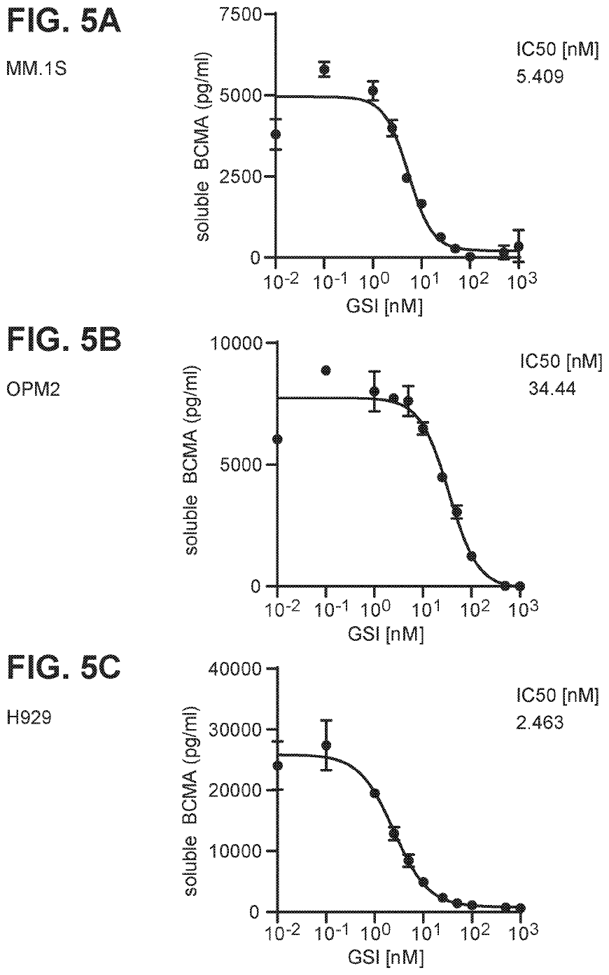

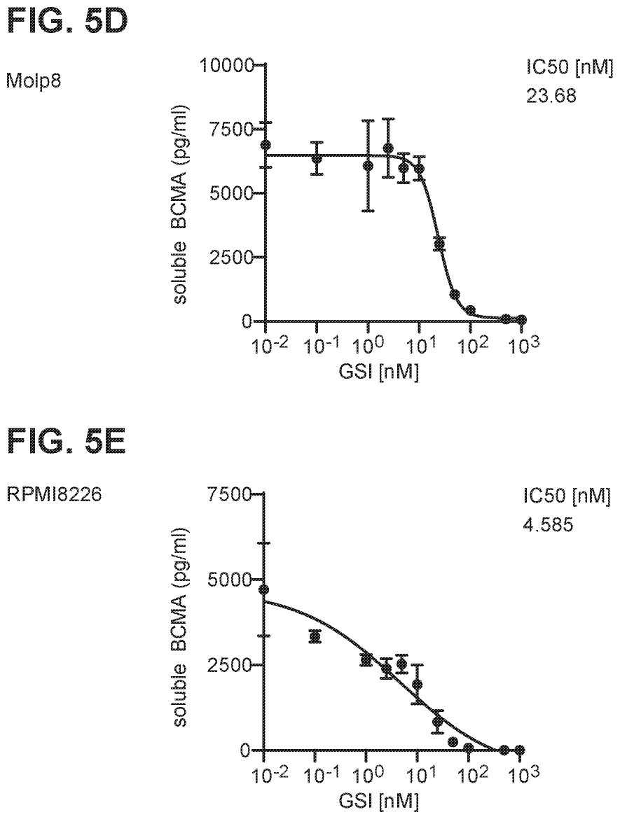

The results summarized in FIG. 5A-5E and Table 6 show that GSI treatment blocks shedding of sBCMA in multiple myeloma cell lines MM.1S, OPM2, H929, Molp8 and RPMI8226, respectively.

TABLE 6
Mean Florescence Intensity ± Standard Deviation

| GSI | BCMA (-PE) MFI | | | | |
|---|---|---|---|---|---|
| [nM] | MM.1S | OPM2 | H929 | Molp8 | RPMI8226 |
| 1000 | 350 ± | 0 | 625.1 ± | 51.6 ± | 0 ± 0 |
| | 494.9 | | 28.7 | 19.2 | |
| 500 | 151.8 ± | 23.8 ± | 716.5 ± | 87.3 ± | 0 ± 0 |
| | 214.7 | 33.7 | 40.2 | 32.2 | |
| 100 | 21.3 ± | 1238.6 ± | 1110.6 ± | 431.8 ± | 64.1 ± |
| | 30.2 | 70.3 | 46.4 | 28.2 | 28.3 |
| 50 | 279.9 ± | 3055.7 ± | 1460.7 ± | 1044.3 ± | 249.5 ± |
| | 32.6 | 267.3 | 55.6 | 161.3 | 99.2 |
| 25 | 630.5 ± | 4487.9 ± | 2336.4 ± | 3021.7 ± | 836.5 ± |
| | 33.2 | 76.6 | 10.3 | 244.1 | 339.2 |
| 10 | 1661.5 ± | 6488.6 ± | 4901.7 ± | 5958.9 ± | 1926.2 ± |
| | 114.7 | 254.6 | 9.3 | 461.5 | 569.7 |
| 5 | 2453.6 ± | 7617.3 ± | 8438.2 ± | 5989.3 ± | 2525.6 ± |
| | 36 | 624.4 | 1019.6 | 566.5 | 267.3 |
| 2.5 | 3988.3 ± | 7726.5 ± | 12874.8 ± | 6765.5 ± | 2398 ± |
| | 251.6 | 64.5 | 1086.7 | 1147.3 | 289.5 |
| 1 | 5143.1 ± | 8014 ± | 19516.4 ± | 6072.9 ± | 2647.5 ± |
| | 296.4 | 815.1 | 615.8 | 1772.4 | 158.6 |
| 0.1 | 5797.5 ± | 8872.2 ± | 27414.8 ± | 6363.4 ± | 3333.8 ± |
| | 228.2 | 24.9 | 4090.6 | 628.8 | 171.3 |
| 0.01 | 3794.2 ± | 6043.3 ± | 24084.8 ± | 6887.3 ± | 4710.8 ± |
| | 471.1 | 41.7 | 3954.7 | 879.1 | 1361.5 |
| 0 | 4276.9 ± | 5948.9 ± | 24399.1 ± | 6859.8 ± | 3333.8 ± |
| | 277.1 | 288.1 | 658.2 | 541.7 | 440.4 |

### Example 6: BCMAxCD3 Bispecific Antibody in Combination with GSI in Multiple Myeloma

This example illustrates that treatment with BCMAxCD3 bispecific antibody in combination with GSI shows enhanced cell killing in multiple myeloma cells cultured with human T cells as compared to BCMAxCD3 bispecific antibody alone.

CD3⁺ T cells from PBMCs (Stem Cell Technologies) were negatively selected using EasySep Human T cell enrichment kit (Stem Cell Technologies). Multiple myeloma cells expressing luciferase (MM.1S-luc, OPM2-luc, H929-luc, Molp8-luc, RPM18226-luc) were treated with 1 μM GSI 10,000. After 24 hours, cells were seeded with 50,000 CD3⁺ pan T cells in a clear 96-well V-bottom plate. Cells were further treated with a range of BCMAxCD3 bispecific antibody concentrations with or without 1 μM GSI. 60 hours after treatment, luciferase activity in treated cells was analyzed using NeoLite reagent kit (Perkin Elmer) and acquired on VictorX multimode plate reader (Perkin Elmer). Cell viability was calculated by dividing luciferase activity of treated cells over luciferase activity of untreated control (no BCMAxCD3 bispecific antibody added).

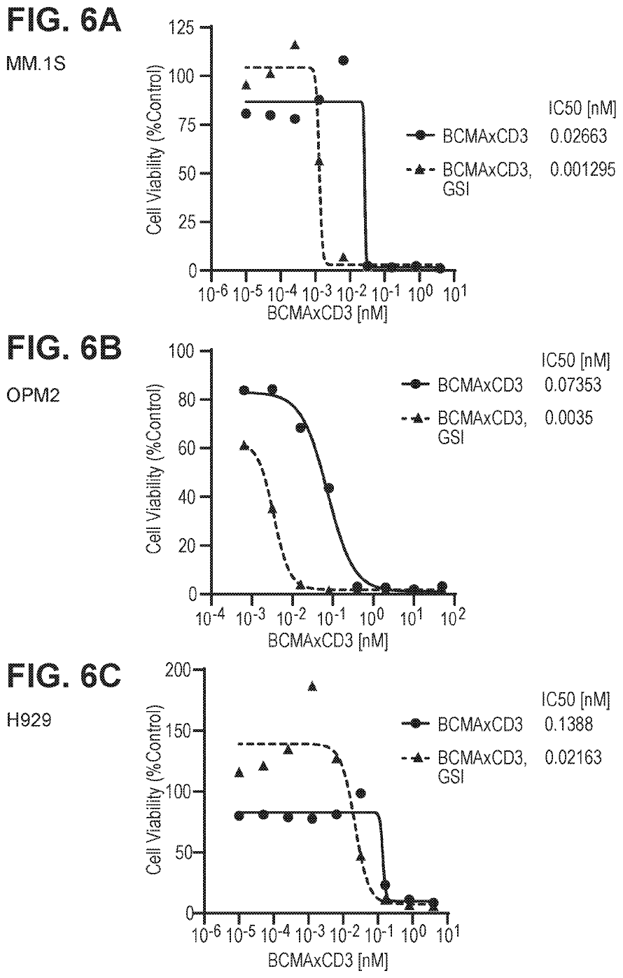

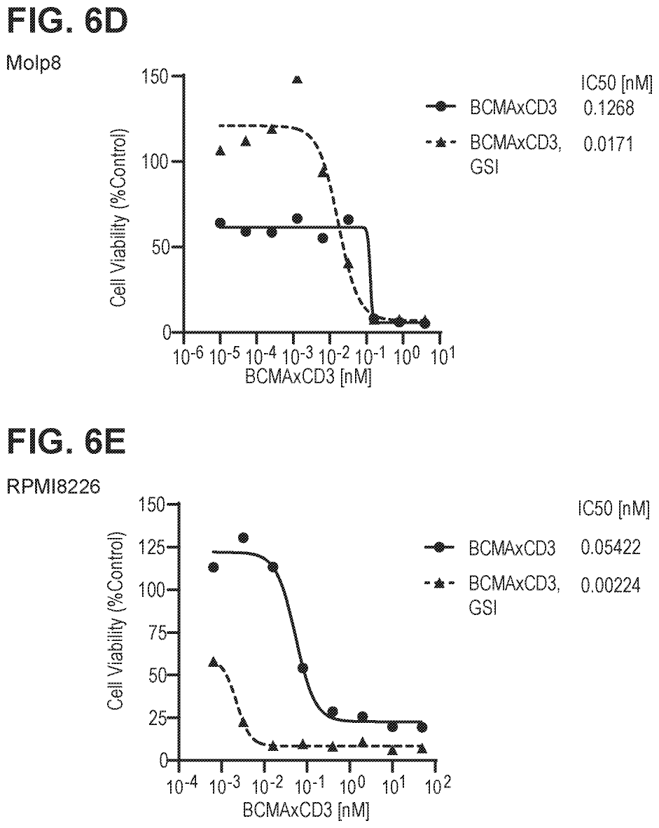

The results summarized in FIG. 6A-6E and Tables 7-8 show that treatment with GSI enhances BCMAxCD3 bispecific antibody (“BCMAxCD3” in Tables 7 and 8) mediated cell killing in multiple myeloma cell lines (MM.1S (21×), OPM2 (21×), H929, Molp8, RPMI8226 (24×), respectively) when cultured with human T cells.

TABLE 7
Mean Florescence Intensity ± Standard Deviation

| | MM.1S cell viability | | H929 cell viability | | Molp8 cell viability | |
|---|---|---|---|---|---|---|
| | (% control) | | (% control) | | (% control) | |
| BCMA × CD3 | | BCMA × CD3 + | | BCMA × CD3 + | | BCMA × CD3 + |
| [nM] | BCMA × CD3 | GSI | BCMA × CD3 | GSI | BCMA × CD3 | GSI |
| 4 | 1.3 ± 0.8 | 1.7 ± 2.1 | 8.6 ± 3 | 6 ± 1 | 5.2 ± 0.3 | 7.3 ± 0.3 |
| 0.8 | 2.4 ± 0.5 | 2 ± 1.5 | 11.3 ± 1.5 | 6.8 ± 2.3 | 6.1 ± 0.7 | 7.6 ± 0.2 |
| 0.16 | 1.7 ± 0.8 | 2 ± 0.3 | 23.2 ± 7.2 | 11.2 ± 4.1 | 8 ± 2 | 7.6 ± 4.3 |
| 0.032 | 2.5 ± 0.3 | 2.1 ± 0.2 | 98.6 ± 1.2 | 47.2 ± 2.5 | 66.1 ± 4.9 | 40.7 ± 34.1 |
| 0.0064 | 108 ± 4.5 | 7.2 ± 4.1 | 81.2 ± 3 | 127.5 ± 9.5 | 55.2 ± 1.3 | 94 ± 9.1 |
| 0.00128 | 87.7 ± 4.4 | 56.6 ± 0.6 | 77.8 ± 3.3 | 186.9 ± 6.5 | 66.7 ± 0.3 | 149 ± 6.3 |
| 0.00026 | 78 ± 0.9 | 116.2 ± 2.6 | 79 ± 0.8 | 134.9 ± 6.8 | 58.5 ± 0.6 | 119.4 ± 4.4 |
| 0.00005 | 79.8 ± 2.9 | 101.5 ± 15.3 | 81.1 ± 0.2 | 121.5 ± 0.9 | 59.1 ± 4.6 | 112.3 ± 0.6 |
| 0.00001 | 80.6 ± 3.9 | 95.6 ± 5.7 | 80.2 ± 0.3 | 116.1 ± 3.3 | 64 ± 11.6 | 106.7 ± 3.1 |

TABLE 8
Mean Florescence Intensity ± Standard Deviation

| BCMAxCD3 | OPM2 cell viability (% control) | | RPMI8226 cell viability (% control) | |
|---|---|---|---|---|
| [nM] | BCMAxCD3 | BCMAxCD3 + GSI | BCMAxCD3 | BCMAxCD3 + GSI |
| 50 | 3.3 ± 0.3 | 2.1 ± 0.9 | 19.4 ± 5 | 7.3 ± 2 |
| 10 | 2 ± 0.2 | 1.9 ± 0.2 | 19.9 ± 1.1 | 6.3 ± 0.2 |
| 2 | 2.8 ± 0.8 | 1.9 ± 0.5 | 25.7 ± 5.5 | 10.9 ± 2.3 |
| 0.4 | 3.1 ± 0.8 | 1.9 ± 0.3 | 28.7 ± 13.6 | 8.4 ± 1.5 |
| 0.08 | 43.6 ± 6.4 | 1.7 ± 0.3 | 54.2 ± 16.9 | 9.8 ± 1.5 |
| 0.016 | 68.4 ± 2.4 | 4 ± 1.5 | 113.4 ± 1.3 | 8.8 ± 2.7 |
| 0.0032 | 84.3 ± 3.1 | 35.3 ± 2.5 | 130.5 ± 19.5 | 22.7 ± 0.9 |
| 0.00064 | 83.8 ± 3.4 | 61.3 ± 2.1 | 113.2 ± 10.6 | 58 ± 6.3 |

### Example 7: In Vitro Study to Detect Cell Surface BCMA Expression in Lymphoma Cell Lines Treated with GSI

This example illustrates upregulation of cell surface BCMA expression lymphoma cells treated with GSI.

Lymphoma cells (Raji cell line) were seeded in a 96-well U bottom plate at 40,000 cells/well. Cells were incubated for 24 hours in the presence of GSI diluted in RPMI medium (0.1% DMSO). The following concentrations of the GSI were tested: 1000 nM, 500 nM, 100 nM, 50 nM, 25 nM, 10 nM, 5 nM, 2.5 nM, 1 nM, 0.1 nM, 0.01 nM. 24 hours later cells were collected and washed with PBS+2% FBS, followed by staining with ZombieNIR Viability Dye (Biolegend) at 1 in 500 dilution in PBS at room temperature for 20 minutes. Next, cells were washed with PBS+2% FBS and stained with anti-BCMA PE-labelled antibody (Biolegend) diluted in PBS+2% FBS for 30 minutes at 4° C. Cells were acquired on BD Flow Cytometer and analyzed using FlowJo flow cytometry analysis software. Dead cells were removed from the analysis by gating on ZombieNIR negative population. BCMA MFI was plotted against GSI concentration to establish EC50.

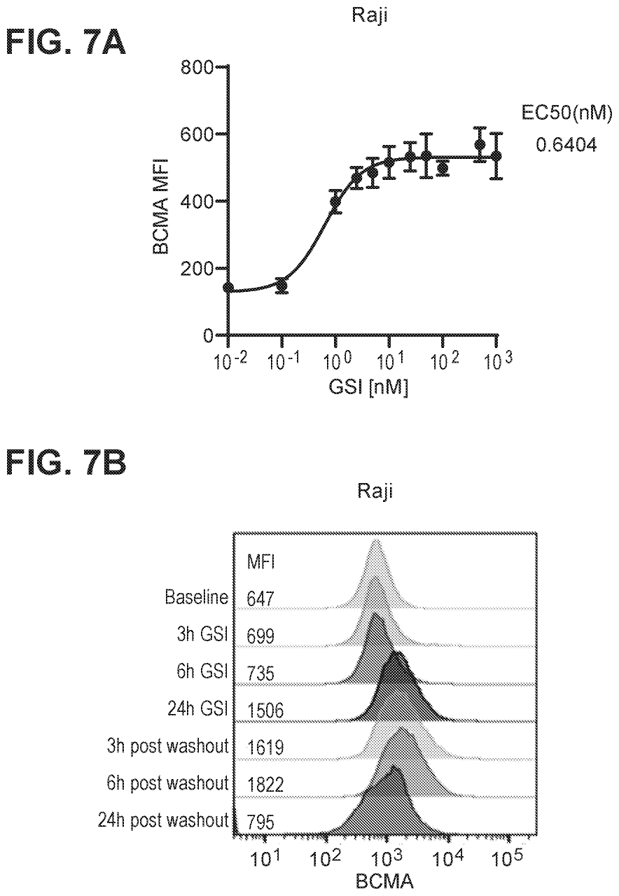

The results summarized in FIG. 7 and Table 9 show that GSI treatment upregulates BCMA expression on the cell surface of Raji lymphoma cells.

TABLE 9
Mean Florescence Intensity ± Standard Deviation

| GSI [nM] | BCMA MFI |
|---|---|
| 1000.00 | 534.5 ± 67.2 |
| 500.00 | 568.5 ± 50.3 |
| 100.00 | 498.5 ± 20.6 |
| 50.00 | 535 ± 65.1 |
| 25.00 | 532 ± 42.5 |
| 10.00 | 515.5 ± 47.4 |
| 5.00 | 484 ± 43.9 |
| 2.50 | 469 ± 31.2 |
| 1.00 | 398 ± 34 |
| 0.10 | 147.5 ± 20.6 |
| 0.01 | 142.5 ± 0.8 |
| 0.0 | 141 ± 4.3 |

### Example 8: In Vitro Study to Detect Cell Surface BCMA Expression in a Time-Dependent Manner in Lymphoma Cell Lines Treated with GSI

This example illustrates that treating lymphoma cell lines with GSI increases BCMA cell surface expression in a time-dependent manner and that BCMA surface levels return to baseline after GSI is removed from the cultures.

Lymphoma cells (Raji) were seeded in a 6-well plate at 800,000 cells/2 ml/well with GSI diluted at 1 μM in RPMI medium (with 0.1% DMSO). Cells were collected to evaluate cell surface BCMA expression at baseline, and then at 3 hours, 6 hours and 24 hours after addition of the GSI. After 24 hours of incubation with the GSI, cells were washed twice in PBS and re-plated in fresh 6-well plates. Cells were further collected for staining at 3 hours, 6 hours and 24 hours after the GSI was washed out. At the indicated time points, samples were stained with a ZombieNIR Viability dye (Biolegend) for 20 minutes at room temperature at 1 in 500 dilution in PBS, washed with PBS+2% FBS and further stained with anti-BCMA PE-labelled antibody diluted in PBS+2% FBS for 30 minutes at 4° C. Samples were acquired on BD Flow Cytometer and analyzed using FlowJo software. Dead cells were removed from the analysis by gating on ZombieNIR negative population. BCMA mean fluorescence intensity (MFI) was plotted as a histogram.

The results summarized in FIG. 7B and Table 10 show that GSI upregulates cell surface BCAM expression on Raji cells in a time-dependent manner and that upregulated surface BCMA expression does not persist after removing GSI from the cultures.

TABLE 10
Mean Florescence Intensity ± Standard Deviation

| Time | BCMA (-PE) MFI |
|---|---|
| Point | Raji |
| Baseline | 647 |
| 3 h after GSI | 699 |
| 6 h after GSI | 735 |
| 24 h after GSI | 1506 |
| 3 h post wash | 1619 |
| 5 h post wash | 1822 |
| 24 h post wash | 795 |

### Example 9A: BCMAxCD3 Bispecific Antibody in Combination with GSI in Lymphoma Cells

This example illustrates that treatment with BCMAxCD3 bispecific antibody in combination with GSI shows enhanced cell killing in low-BCMA expressing lymphoma cells cultured with human T cells as compared to BCMAxCD3 bispecific antibody alone.

CD3⁺ T cells from PBMCs (Stem Cell Technologies) were negatively selected using EasySep Human T cell enrichment kit (Stem Cell Technologies). 10,000 target lymphoma cells expressing luciferase (Raji-luc) were treated with 1 μM GSI. After 24 hours, cells were seeded with 50,000 CD3⁺ pan T cells in a clear 96-well V-bottom plate. Cells were further treated with a range of BCMAxCD3 bispecific concentrations with or without 1 μM GSI. 60 hours after treatment, luciferase activity in treated cells was analyzed using NeoLite reagent kit (Perkin Elmer) and acquired on VictorX multimode plate reader (Perkin Elmer). Cell viability was calculated by dividing luciferase activity of treated cells over luciferase activity of untreated control (no BCMAxCD3 bispecific antibody added).

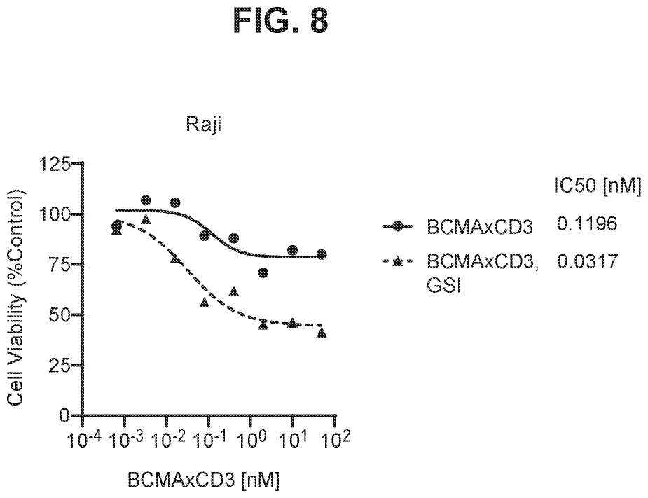

The results summarized in FIG. 8 and Table 11A show that treatment with GSI enhances BCMAxCD3 bispecific antibody mediated cell killing in lymphoma cell line (Raji) when cultured with human T cells.

TABLE 11A
Cell Viability ± Standard Deviation

| | Raji Cell Viability | |
|---|---|---|
| BCMAxCD3 | (% control) | |
| [nM] | BCMAxCD3 | BCMAxCD3 + GSI |
| 50 | 80 ± 13.2 | 41.5 ± 9.3 |
| 10 | 82.1 ± 9.7 | 46.4 ± 14.1 |
| 2 | 71 ± 9.7 | 45.3 ± 4.3 |
| 0.4 | 88.1 ± 18.4 | 61.9 ± 3 |
| 0.08 | 89.3 ± 5.7 | 56.3 ± 0.3 |
| 0.016 | 105.8 ± 10.2 | 78.2 ± 9.1 |
| 0.0032 | 107 ± 4.5 | 97.8 ± 8.2 |
| 0.00064 | 94 ± 3.7 | 92.4 ± 10.8 |

### Example 9B: Gamma Secretase Inhibitor Action Increases In Vitro Cytotoxic Effect of BCMAxCD3 Bispecific Antibody PF06863135 (Elranatamab) on Multiple Myeloma Cells in Co-Culture Assay

This example illustrates the combination benefit of treating multiple myeloma cells with GSI and BCMAxCD3 bispecific antibody PF06863135 (elranatamab) compared to the BCMAxCD3 antibody alone in cytotoxic T lymphocyte (CTL) in vitro co-culture assays.

Multiple myeloma cell lines expressing luciferase (H929-Luc, Molp8-Luc, OPM2-Luc, and RPMI8226-Luc) were cultured at 37° C. and 5% CO₂ with 1 mM GSI for 24 hr or left untreated. Myeloma cells were then harvested and transferred onto 96-well U-bottom plates at 10,000 cells/well along with 50,000 CD3⁺ T cells/well, which were enriched from human PBMC using a negative selection Pan T Cell Isolation kit (Miltenyi Biotec). Media with or without 1 mM GSI and containing serial dilutions of BCMAxCD3 bispecific PF06863135 were further added to wells before incubating plates at 37° C. and 5% CO₂ for 72 hr. At the end of the incubation period, Bright-Glo substrate (Promega) was added to wells and luminescence measured on a SpectraMax plate reader. Percent cell viability was calculated by taking the luminescence signal value for each test well, dividing by the average signal from no antibody treatment control wells and then multiplying by 100. EC₅₀ values were further calculated by generating a four-parameter dose-response curve fit of cell viability data vs. antibody dose concentration using GraphPad Prism. Table 11B shows that treatment with GSI improves BCMAxCD3 antibody-mediated killing of multiple myeloma cells (H929, Molp8, OPM2, and RPMI8226) treated in co-cultures with human T cells.

TABLE 11B
BCMA × CD3 Bispecific Antibody PF06863135
mediated Killing of Multiple Myeloma Cells

| | | | | | | | Fold |
|---|---|---|---|---|---|---|---|
| | BCMA × CD3 | | | BCMA × CD3 + GSI | | | increase |
| | EC₅₀ | | | EC₅₀ | | | EC₅₀ with |
| Cell line | (nM) | SD | N | (nM) | SD | N | GSI |
| H929-Luc | 0.1668 | 0.1126 | 6 | 0.0055 | 0.0042 | 6 | 30.3× |
| Molp8-Luc | 0.1085 | 0.0456 | 6 | 0.0202 | 0.0177 | 6 | 5.4× |
| OPM2-Luc | 0.0528 | 0.0122 | 6 | 0.0033 | 0.0008 | 6 | 15.9× |
| RPMI8226- | 0.0092 | 0.0065 | 6 | 0.0017 | 0.0008 | 6 | 5.5× |
| Luc | | | | | | | |

### Example 10: First in Human Phase 1 Clinical Study of BCMAxCD3 Bispecific Antibody Elranatamab (PF-06863135)

This Example illustrates an ongoing Phase 1 open-label, multicenter clinical study of PF-06863135 (BCMAxCD3 bispecific) as monotherapy and in combination with sasanlimab, lenalidomide or pomalidomide, in adult patients with advanced multiple myeloma who have relapsed from or are refractory to standard therapy. The study has been registered on ClinicalTrials.gov with identifier NCT03269136 and was first posted August 2017. Study results for Part 1 of the trial and additional arms for the study are described in this Example.

The study arms and the initial dosing designs are briefly described in Table 12. For each of the study arms, treatment with the drugs will continue until disease progression, patient refusal (withdrawal of consent) or unacceptable toxicity occurs.

TABLE 12
PF-06863135 First In Human Clinical Study Treatments

| Study | | |
|---|---|---|
| Arms | Drugs | Dose levels and Schedule |
| Part 1 | Single agent PF06863135, | IV: 0.1, 0.3, 1, 3, 10, 30, and 50 |
| | | (μg/kg) Q1W; |
| | | SC: 80, 130, 215, 360, 600 and |
| | | 1000 (μg/kg), Q1W; |
| | | Selected doses from the above for |
| | | Q2W |
| Part 1.1 | Single agent PF-06863135, | Doses in Part 1, plus a priming dose |
| | | at cycle zero day 1. Subsequently |
| | | set at 600 (μg/kg) priming followed |
| | | by 1000 (μg/kg) Q1W; or 600 |
| | | (μg/kg) priming followed by 1000 |
| | | (μg/kg) Q2W |
| Part 1B | PF-06863135 and | PF-06863135: to be determined |
| | sasanlimab | Sasanlimab: 300 mg Q4W, SC |
| Part 1C | PF-06863135 and | PF-06863135: to be determined |
| | lenalidomide | Lenalidomide: 25 mg orally daily on |
| | | days 1-21 without dexamethasone |
| | | Subsequently modified to 15 mg |
| | | QD day 1-21 |
| Part 1D | PF-06863135 and | PF-06863135: to be determined |
| | pomalidomide | Pomalidomide: 4 mg orally daily on |
| | | days 1-21 without dexamethasone |
| Part 2A | PF-06863125 single agent | RP2D, to be determined |
| Part 2B | PF-06863135 and | PF-06863135: to be determined |
| | sasanlimab | Sasanlimab: 300 mg Q4W, SC |
| Part 2C | PF-06863135 and | PF-06863135: to be determined |
| | lenalidomide | Lenalidomide: 25 mg orally daily on |
| | | days 1-21 (without dexamethasone) |
| Part 2D | PF-06863135 and | PF-06863135: to be determined |
| | pomalidomide | Pomalidomide: 4 mg orally daily on |
| | | days 1-21 (without dexamethasone) |

Subsequently the RP2D dose was determined based on clinical outcome of Part 1 and was chosen to be a maintenance dosing of 76 mg Q1W SC with a single priming dose of 44 mg SC administered one week prior to the first maintenance dose.

In Part 1 Combination dose finding, it was decided that the subject will be administered fixed doses of PF06863135, with maintenance doses starting one week after the priming dose, and the starting dose is one level below the single agent RP2D, and to be escalated to the RP2D doses or deescalated to the RP2D minus 2 level. Table 12A describes the potential fixed dose levels in a combination study of PF06863135 with a second therapeutic agent. For Part 1C, the starting dose of lenalidomide was modified to 15 mg QD oral day 1-21 in a 28 days cycle starting 7 days after the priming dose of PF06863135.

TABLE 12A
Potential Fixed Dose Levels for Combination Studies

| Dose Level | Priming Dose (mg) | Maintenance Dose (mg) |
|---|---|---|
| PR2D Minus 2 | 24 | 32 |
| RP2D Minus 1 | 32 | 44 |
| RP2D dose | 44 | 76 |

Part 1 of the Study is the PF-06813135 single agent dose escalation arm at the dose levels of 0.1, 0.3, 1, 3, 10, 30, and 50 μg/kg Q1W by intravenous (IV) administration, and at the dose levels of 80, 130, 215, 360, 600 and 1000 μg/kg Q1W by subcutaneous (SC) administration. Upon reaching maximum tolerated dose (MTD)/maximum administered dose (MAD), patients can be treated at a dose level selected from the foregoing dose levels described in this paragraph, and below the MTD/MAD fora Q2W administration, both IV and SC, to further support the recommended phase 2 dose (RP2D) decision. For the study, dose limiting toxicity observation period is set at 21 days for Q1W dosing and 28 days for Q2W dosing. The treatment cycle, aka cycle, for Q1W dosing would be 3 weeks, and for Q2W dosing would be four weeks.

Clinical Outcome of Part 1 of the Study. As of 15 Apr. 2020, a total of 23 patients were enrolled in Part 1 of the study and had been treated with PF-06863135 administered intravenously (IV) at 0.1 (N=2), 0.3 (N=3), 1 (N=2), 3 (N=3), 10 (N=2), 30 (N=5), and 50 (N=6) μg/kg. As of Aug. 21, 2020, a total of 30 patients were enrolled in Part 1 of the study and had been treated with PF-06863135 administered subcutaneously (SC) at 80 (N=6), 130 (N=4), 215 (N=4), 360 (N=4), 600 (N=6) and 1000 (N=6) μg/kg. Safety and efficacy data were available for in 23 IV and 30 SC treated patients per IMWG (International Myeloma Working Group) criteria.

Of the patients in the IV cohorts, 2 patients (1 patient in the 30 and 1 patient in the 50 μg/kg cohort) experienced dose limiting toxicities (DLTs) of febrile neutropenia Grade 3 and electrocardiogram QT prolongation Grade 1. No patients in the SC cohorts experienced DLTs. Cytokine release syndrome (CRS) was the most common adverse event reported. In the IV cohorts, CRS was observed in 1 (50.0%), 4 (80.0%), and 6 (100.0%) patient(s) in the 10, 30, and 50 μg/kg cohorts. Out of all IV treated patients, 6 (26.1%) experienced maximum Grade 1 CRS while 5 (21.7%) experienced maximum Grade 2 CRS. CRS began within first 2 days of dosing for each of the 11 patients with CRS. In 3 patients at 50 μg/kg, CRS also occurred after the second dose for 1 patient, after the second and third dose for 1 patient, and after the third and fourth dose for 1 patient.

In the SC cohorts, CRS was observed in 3 (50.0%), 2 (50.0%), 3 (75.0%), 3 (75.0%), 6 (100%) and 6 (100%) of patients in the 80, 130, 215, 360, 600 and 1000 μg/kg groups, respectively. Out of all SC treated patients, 18 (60.0%) experienced maximum Grade 1 CRS while 5 (16.7%) experienced maximum Grade 2 CRS. CRS primarily began within first 2 days of dosing. Table 13 describes further details of the CRS in the SC cohorts.

TABLE 13
Cytokine Release Syndrome (CRS) in SC Cohorts Part 1 and Part 1.1 of the Study

| | | | | | | | 600 (μg/kg) |
|---|---|---|---|---|---|---|---|
| | 80 | 130 | 215 | 360 | 600 | 1000 | priming, |
| | μg/kg | μg/kg | μg/kg | μg/kg | μg/kg | μg/kg | 1000 (μg/kg) |
| | Q1W | Q1W | Q1W | Q1W | Q1W | Q1W | Q1W or Q2W |
| | N = 6 | N = 4 | N = 4 | N = 4 | N = 6 | N = 6 | N = 20 |
| Patients | 3 (50%) | 2 (50%) | 3 (75%) | 3 (75%) | 6 (100%) | 6 (100%) | 20 (100%) |
| with CRS | | | | | | | |
| Grade 1 | 2 | 2 | 3 | 2 | 4 | 5 | 11 |
| Grade 2 | 1 | 0 | 0 | 1 | 2 | 1 | 9 |
| Mean | 1 day | 3 days | 3 days | 1.7 days | 1.7 days | 4 days | 2 Days |
| duration | | | | | | | |

In the IV cohorts, 2 patients achieved a minimal response at 3 μg/kg and 50 μg/kg IV, and 1 patient achieved a complete response at 50 μg/kg IV. Ten subjects in the IV cohorts (0.3-50 μg/kg) achieved best response of stable disease.

In the SC cohorts, efficacy results are summarized in below Table 14.

TABLE 14
Patient Response in the SC cohorts of Part 1, and Part 1.1 of the Study

| | | | | | 600 (μg/kg) | 600 (μg/kg) |
|---|---|---|---|---|---|---|
| | 215 | 360 | 600 | 1000 | priming, 1000 | priming, 1000 |
| | μg/kg | μg/kg | μg/kg | μg/kg | (μg/kg) Q1W | (μg/kg) Q2W |
| | N = 4 | N = 4 | N = 6 | N = 6 | N = 7 | N = 13 |
| OR | 3/4 (75%) | 3/4 (75%) | 4/6 (67%) | 5/6 (83%) | 4/7 (57%) | 7/13 (54%) |
| sCR | 2 | 1 | 2 | | | |
| CR | | | | 1 | 2 | |
| VGPR | | 2 | 2 | 3 | 1 | 4 |
| PR | 1 | | | 1 | 1 | 3 |
| SD | 1 | | 1 | | | 1 |
| PD | | 1 | 1 | 1 | | 3 |

These results show that at the highest dose levels of 600 and 1000 μg/kg SC, clinical efficacy was seen in most patients, and toxicities were tolerable and manageable, with less severe CRS occurring in SC-treated patients despite higher overall dose exposure in SC-treated vs IV-treated patients.

Part 1.1 of the Study is an alternative maintenance dose escalation arm for single agent PF-06863135. If excessive toxicity occurs or the maximum tolerated dose (MTD)/maximum administered dose (MAD) is reached at a dose level that is earlier than desired in Part 1 of the study described above, a priming dose will be administered one week prior to the day 1 cycle 1 administration of the dose (maintenance dose) at this dose level and for all subsequent dose levels in the dose escalation that may be initiated for Part 1.1. The priming dosing will be at a lower dose level than the maintenance dose.

Clinical outcome of Part 1.1 of the Study. As of Feb. 4, 2021, a total of 20 patients were enrolled and treatment in Part 1.1 of the Study, with cohort of 7 patients at a 600 μg/kg priming dose followed by 1000 μg/kg Q1W dosing, and a cohort of 13 patients at a 600 μg/kg priming dose followed by 1000 μg/kg Q2W dosing. The CRS in these two cohorts is described in Table 13. Introducing of priming dose decreased medium duration of CRS by 50%, from 4 days to 2 days. The dose frequency in part 1.1 of the study (Q1W v. Q2W) had no impact on the CRS. Patient response of Part 1.1 of the Study is described in Table 14.

Part 2A of the study is the single agent PF-06863135 dose expansion arm. Based on single agent dose escalation clinical data, either IV or SC administration, including priming and maintenance dose, and either Q1W or Q2W dosing will be selected for Part 2A of the study. In particular, SC administration at a dose level of 215, 360, 600 or 1000 μg/kg at Q1W or Q2W, without a priming dose, or SC administration at a maintenance dose level of 215, 360, 600 or 1000 μg/kg at Q1W or Q2W, with a priming dose at day 1 cycle zero at a dose level less than that of the maintenance dose, is looking promising as the RP2D for the phase 2A study.

Preliminary pharmacokinetic (PK) analysis indicated that body weight is not a clinically relevant factor on PF-06863135 exposure. Therefore, a fixed dose is suitable for the dosing of PF-06863135. Based on the encouraging efficacy data and safety data obtained from Part I of Study, a promising RP2D for Part 2A of the study could be a fixed dose equivalent of 1000 μg/kg (i.e., 76 mg) of PF-06863135 either in Q1W or Q2W. A fixed dose equivalent of 600 μg/kg (ie, 44 mg) will likely be used as a priming dose on cycle zero day 1. The initial dose of 44 mg serves as a priming dose and is designed to mitigate the CRS symptoms of the later 76 mg dose. Based on the results of Part 1 of the Study, CRS primarily occurs after the initial dose. Subsequently, the 44 mg (priming) and 76 mg (maintenance) were selected as the single agent RP2D dose. Patient will be administered a single priming dose of 44 mg SC of PF06863135, followed by maintenance dosing of 76 mg Q1W SC or 76 mg Q2W starting 7 days after the single priming dose.

Part 1B and Part 2B of the Study are combination therapy of PF-06863135 and sasanlimab, a PD-1 antibody. The treatment cycle is 28 days. Sasanlimab will be administered at 300 mg SC Q4W, starting at day 1, cycle 1. PF-06863135 will be administered SC or IV on a selected dose, Q1W or Q2W, starting on day 1, cycle 1, with or without a priming dose one week prior to day 1 cycle 1.

In Part 1B, the dose of PF-06863135 will be determined based on the results of Part 1 and Part 1.1 of the study, starting at the RP2D described for above Part 2A of the study, or at the MTD/MAD minus one level, whichever is lower. If the combination regimen is not well tolerated, de-escalation of the PF-06863135 to a lower dose level will be conducted to choose the dose level for Part 2B.

In Part 2B, the PF06863135 will be administered at a dose level based on the results of Part 1B.

Part 1C and Part 2C of the Study are combination therapy of PF-06863135 and lenalidomide. The treatment cycle is 28 days. Lenalidomide will be administered daily on days 1-21 at 25 mg oral (PO) without dexamethasone, starting at day 1, cycle 1. PF-06863135 will be administered SC or IV on a selected dose, Q1W or Q2W, starting on day 1, cycle 1, with or without a priming dose one week prior to day 1 cycle 1.

In Part 1C, the dose of PF-06863135 will be determined based on the results of Part 1 and Part 1.1 of the study, and the initial plan was starting at the RP2D described for above Part 2A of the study, or at the MTD/MAD, whichever is lower. If the combination regimen is not well tolerated, de-escalation of the PF-06863135 to a lower dose level will be conducted to choose the dose level for Part 2C. Subsequently it was decided to start at the dose level of PF06863135 that is one level below the single agent RP2D as described in Table 12A. The starting dose of lenalidomide was modified to 15 mg QD oral day 1-21 in a 28 days cycle starting 7 days after the priming dose of PF06863135.

In Part 2C, the PF-06863135 will be administered at a dose level based on the results of Part 1C.

Part 1D and Part 2D of the Study are combination therapy of PF06863135 and pomalidomide. The treatment cycle is 28 days. Pomalidomide will be administered daily on days 1-21 at 4 mg PO without dexamethasone, starting at day 1, cycle 1. PF-06863135 will be administered SC or IV on a selected dose, Q1W or Q2W, starting on day 1, cycle 1, with or without a priming dose one week prior to day 1 cycle 1.

In Part 1D, the dose of PF-06863135 will be determined based on the results of Part 1 and Part 1.1 of the study, starting at the RP2D described for above Part 2A of the study, or at the MTD/MAD, whichever is lower. If the combination regimen is not well tolerated, de-escalation of the PF06863135 to a lower dose level will be conducted to choose the dose level for Part 2D. Subsequently it was decided to start at the dose level of PF06863135 that is one level below the single agent RP2D as described in Table 12A.

In Part 2D, the PF-06863135 will be administered at a dose level based on the results of Part 1D.

Patient enrollment criteria. For all arms of the study described herein, patient enrollment criteria includes that patients must have progressed on or are intolerant of established therapies known to provide clinical benefit in multiple myeloma including proteasome inhibitor, immunomodulatory imid drugs (ImiD) and an anti-CD38 mAb, where approved and available, either in combination or as a single agent, and that patients must not be candidate for regimens known to provide clinical benefit in relapse or refractory multiple myeloma based on the investigator's judgement.

Primary and secondary objections of the study include (1) to assess preliminary clinical efficacy at PF-06863135 RP2D, (2) to further characterize the safety and tolerability, (3) to evaluate PK of PF-06863135 at RP2D, (4) to evaluate immunogenicity of PF-06863135, (5) to characterize the impact of PF-06863135 on systemic soluble immune factors, each of the (1) through (5) is with regard to PF-06863135 as monotherapy and in combination with sasanlimab, lenalidomide, or pomalidomide.

### Example 11. Phase 2 Clinical Study of BCMAxCD3 Bispecific Antibody PF-06863135 Monotherapy in Participants with Multiple Myeloma Who are Refractory to at Least One Proteasome Inhibitor, One IMiD and One Anti-CD38 Monoclonal Antibody

This study is an open-label, multicenter, non-randomized, Phase 2 study to evaluate the efficacy and safety of PF-06863135 in refractory/relapsed multiple myeloma (RRMM) participants who are refractory to at least one proteasome inhibitor (PI), one IMiD, and one anti-CD38 mAb. To determine the effects of prior BCMA-directed therapy on the response to PF-06863135 monotherapy, this study will enroll 2 independent and parallel cohorts, one with participants who are naïve to BCMA-directed therapies (Cohort A; approximately 90 participants) and the other with participants who have received prior BCMA directed ADC or BCMA directed CAR T-cell therapy, either approved or investigational (Cohort B; approximately 60 participants). The primary objective for each independent cohort will be to determine the efficacy (i.e. ORR) of PF-06863135 as assessed by blinded independent central review (BICR), as defined by International Myeloma Working Group (IMWG). The study design scheme is shown in below Table 15.

TABLE 15
Study Treatments of Phase 2 Clinical Study of BCMAxCD3
bispecific antibody PF-06863135 Monotherapy

| Cohorts | Patient Number and Prior Therapies | Intervention/Dosing |
|---|---|---|
| A | N = 90; RRMM. Refractory | PF-06863135: 44 mg SC |
| | to all three of | priming dose on C1D1; |
| | IMiD, PI and anti-CD 38; no prior | followed by 76 mg SC |
| | BCMA directed therapy | maintenance dose Q1W |
| B | N = 60; RRMM. Refractory | starting C1D8, and |
| | to all three of IMiD, PI and | optionally switch to 76 mg |
| | anti-CD 38; prior BCMA directed | SC Q2W after 6 cycles |
| | ADC or CAR-T cell therapy, | (each cycle is 28 days) |
| | but no prior BCMA-directed | |
| | bispecific Abs. | |

Dosing: The participant in each cohort will be administered through subcutaneous injection (SC) an initial dose of 44 mg of PF-06863135 on cycle 1 day 1 (C1D1). Each treatment cycle is 28 days. The initial dose of 44 mg serves as a priming dose and is expected to mitigate CRS symptoms, which are mainly expected after the initial dose. The priming dose was later modified to be 12 mg of PF06863135 to be administered on C1D1 followed by 32 mg of PF06863135 to be administered on C1D4. The dose of PF-06863135 should be increased to 76 mg SC Q1W starting on Cycle 1 Day 8 as long as the participant meets all of the three criteria described below:
(1) ANC≥1.0×10⁹/L;
(2) Platelets count≥25×10⁹/L; and
(3) Recovery of treatment-related non-hematologic toxicities to baseline or Grade ≤1 severity (or, at the investigator's discretion, Grade ≤2 if not considered a safety risk for the participant).

If a participant does not meet these criteria on Cycle 1 Day 8, initiation of dosing with 76 mg should be deferred until these criteria are met. If a participant has received Q1W dosing for at least 6 cycles and has achieved IMWG response of a PR or better with responses persisting for at least 2 months, the dose interval should be changed from Q1W to Q2W as a lower dose intensity might be adequate to maintain the response given the reduced disease burden in these participants. However, the participant may remain on the Q1W schedule based on the medical judgement of the investigator and after consultation with the trial sponsor. After changing to Q2W interval, the dosing interval may be returned to Q1W according to the medical judgement of the investigator.

For each of the study cohorts, treatment with PF-06863135 will continue until disease progression, patient refusal (withdrawal of consent) or unacceptable toxicity occurs. The study will be completed when all participants have discontinued study intervention and have been followed for overall survival (OS) for at least 2 years.

Primary endpoints: to determine overall response rate (ORR) by blinded independent central review (BICR) per International Myeloma Working Group (IMWG)

Secondary endpoints: (1) duration of response (DOR) by BICR and investigator per IMWG; (2) cumulative complete response rate (CCRR) by BICR and investigator per IMWG; (3) ORR by investigator per IMWG; (4) duration of cumulative complete response (DOCCR) by BICR and investigator per IMWG; (5) progression free survival (PFS) by BICR and investigator per IMWG; (6) overall survival (OS); (7) time to response (TTR) by BICR and investigator per IMWG; (8) minimum residual disease (MRD) negativity rate (central lab) per IMWG; (9) AEs and laboratory abnormalities as graded by NCI Common Terminology Criteria for Adverse Events (CTCAE) v5.0; (10) Severity of CRS and immune effector cell-associated neurotoxicity syndrome (ICANS) assessed according to American Society for Transplantation and Cellular Therapy (ASTCT) criteria. (11) Pre- and post-dose concentrations of PF-06863135 and (12) ADAs and NAbs against PF-06863135

### Example 12. A Phase 1/2, Open Label, Multicenter Study to Evaluate Two Step-Up Priming Doses and Longer Dosing Intervals of Elranatamab (PF-06863135) Monotherapy in Participants with Relapsed/Refractory Multiple Myeloma

The objectives of this study is to assess the rate of Grade 2 or higher CRS when elranatamab is administered with a dosing regimen of 2 step-up priming doses and premedication. In addition, this study will assess the safety, tolerability, PK and preliminary anti-myeloma activity of elranatamab at doses higher than 76 mg with different dosing intervals (QW, Q2W and Q4W) in participants with Relapsed/Refractory Multiple Myeloma (RRMM). A regimen of elranatamab full dose of 76 mg QW for 6 cycles followed by Q2W (Part 2) will also be assessed. Cycle 1 starts on the day when the first priming dose is administered to the participant.

All doses of elranatamab will be administered subcutaneously (SC).

In the First Cycle (C1) of elranatamab treatment, the following regimen will be evaluated for all participants in the study:

C1D1: premedication+elranatamab 12 mg; C1D4: premedication+elranatamab 32 mg; C1D8: premedication+elranatamab 76 mg; C1D15 and C1D22: elranatamab 76 mg.

Premedications are required approximately 60 minutes prior to both priming doses (on C1D1 and C1D4) and the first full dose of elranatamab (C1D8). Premedication to be used are, acetaminophen 650 mg (or paracetamol 500 mg), diphenhydramine 25 mg, oral or IV, and dexamethasone 20 mg (or equivalent) oral or IV.

For Cycle 2 and onwards, the following will be evaluated: Part 1A. In dose level 1 cohort, the participants will be administered 116 mg Q2W for C2 to C6, optionally switching to 116 mg Q4W for participants with IMWG response of PR or better for at least 2 cycles on Q2W. If Dose level 1 is tolerable, in Dose level 2 cohort, the participants will be administered 152 mg Q2W for C2 to C6, optionally switching to 152 mg Q4W for participants with IMWG response of PR or better for at least 2 cycles on Q2W. For both dose level 1 and dose level 2, if after switching to Q4W interval, the participant subsequently begins to have an increase of disease burden not yet qualifying as PD according to IMWG criteria, dosing interval should return to Q2W at the same dose level (e.g. from 152 mg Q4W to 152 mg Q2W).

Part 1B. It will begin once the potential MTD/RP2D is identified from Part 1A and would be the dose expansion cohort of the selected dose level.

Part 1C. It will only begin if both Dose level 1 and Dose level 2 in Part 1A are tolerable. Here, for C2 to C3, participants will be administered 116 mg Q1W or 152 mg Q1W. For C4 to C6, for participants with IMWG response of PR or better at C2 and C3, 116 mg or 152 mg Q2W will be administered. For C7 and onwards, for participants with IMWG response of PR or better for at least 2 cycles on Q2W, 116 mg or 152 mg Q4W will be administered.

Part 2: 76 mg Q1W will be administered from C2 to C6. For participants with IMWG response of PR or better for at least 2 cycles on Q1W, 76 mg Q2W will be administered for C7 and onwards. If after switching to Q2W interval, the participant subsequently begins to have an increase of disease burden not yet qualifying as PD according to IMWG criteria, dose interval should return to Q1W at 76 mg.

### Example 13. An Open Label Multicenter, Randomized Phase 3 Study to Evaluate the Efficacy and Safety of Elranatamab (PF06863135) and Daratumumab in Participants with Relapsed/Refractory Multiple Myeloma (RRMM)

The objectives of the Part 1 of this study is to assess DLTs, safety and tolerability of elranatamab plus daratumumab to select RP3D for the combination. The objectives for Part 2 is to compare the efficacy of elranatamab (Arm A), and elranatamab plus daratumumab combination (Arm B), each with the control arm combination therapy daratumumab plus pomalidomide plus dexamethasone (Arm C). The objectives of Part 1 of this study also include to assess the rate of Grade 2 and above CRS when elranatamab alone or in combination is administered with 2 steps-up priming doses along with premedication. The study treatments are described in Table 16. A cycle is 28 days.

TABLE 16
Elranatamab and Daratumumab Combination Study Treatments

| Arm | Drugs and Dosing |
|---|---|
| Part 1, Dose | Elranatamab: 12 mg (C1D1) + 32 mg (C1D4) |
| level minus 1 | priming, 44 mg QW (starting C1D8) maintenance, |
| | optionally switched to 44 mg Q2W |
| | after 6 cycles |
| | Daratumumab: 1800 mg SC, QW from C1D15, |
| | Q2W from C3 D15, Q4W from C8D1 |
| Part 1, Dose | Elranatamab: 12 mg (C1D1) + 32 mg (C1D4) |
| Level 1 | priming, 76 mg QW (starting C1D8) maintenance, |
| | optionally switched to 76 mg Q2W |
| | after 6 cycles |
| | Daratumumab: 1800 mg SC, QW from C1D15, |
| | Q2W from C3 D15, Q4W from C8D1 |
| Part 1, Dose | Elranatamab: select Dose level minus 1 or Dose level 1 |
| expansion | Daratumumab 1800 mg SC, QW from |
| | C1D15, Q2W from C3 D15, Q4W from C8D1 |
| Part 2, Arm A | Elranatamab |
| Part 2, Arm B | Elranatamab, dosing TBD based o Part 1 |
| | Daratumumab 1800 mg SC, QW from C1D15, |
| | Q2W from C3 D15, Q4W from C8D1 |
| Part 2, Arm C | Daratumumab, 1800 mg SC, QW from C1D1, |
| | Q2W from C3 D1, Q4W from C7D1 |
| | Pomalidomide: 4 mg PO QD on day 1-21 of each |
| | 28 day cycle; Dexamethasone: 40 mg (20 mg for |
| | participant 75 years and older) PO QW |

Elranatamab dosing: in Part 1, dose level minus 1, after participant have been on 44 QW until the end of cycle 6, 44 mg Q2W should be dosed thereafter for participants with IMWG response of PR or better persisting for at least 2 cycles. Similarly, 76 mg QW is switched to 76 mg Q2W Part 1, in Part 1, dose level 1, and similarly QW will be switched to Q2W in Arm A and Arm B in Part 2. Subsequently, if increase of disease burden (not qualifying as PD according to IMWG criteria) is observed, the dosing interval should return to QW.

Daratumumab dosing: subcutaneous injection of 1800 mg will be used, Q1W, followed by Q2W followed by Q4W in line with the USPI dosing schedule of the FDA approved daratumumab and hyaluronidase-fihj product.

Premedication is required approximately 60 minutes prior to both priming dose (on C1D1 and C4 D1) and the first full dose of elranatamab (C1D8). Premedication is also required 1-3 hours prior to each dose of daratumumab except for Part 2 Arm C wherein the dexamethasone component of the treatment regimen should be administered prior to daratumumab and serve as premedication. If Elranatamab and daratumumab are to be administered on the same day, premedication should be given only once on that day prior to dosing of both elranatamab and daratumumab. Premedication to be used are, acetaminophen 650-1000 mg (or paracetamol 500 mg), diphenhydramine 25-50 mg, oral or IV, or dexamethasone 20 mg (or equivalent) oral or IV.

### Example 14. A Randomized, 2-Arm Phase 3 Study of Elranatamab (PF-06863135) Plus Lenalidomide Versus Lenalidomide in Patients with Newly Diagnosed Multiple Myeloma (NDMM) Who are Minimum Residual Disease (MRD) Positive after Undergoing Autologous Stem-Cell Transplantation (ASCT)

The objectives of this study include to compare the efficacy of elranatamab plus lenalidomide combination therapy (arm A) with lenalidomide (arm B) and to determine the safety and tolerability of elranatamab plus lenalidomide combination therapy. Participants in the study will be those with newly diagnosed multiple myeloma who are minimum residual disease (MRD) positive after undergoing autologous stem-cell transplantation. Table 16 below describes the planned dosing regimens for each arm of the study.

TABLE 16
Combination Therapy of Elranatamab plus Lenalidomide
versus Lenalidomide for NDMM Patients who are MRD Positive
after ASCT Study Treatments

| Arms | Drugs and Dosing |
|---|---|
| A | Elranatamab: C1D1 priming dose of 12 mg; C1D4 priming dose of 32 |
| | mg; C1D8 full dose (to be determined,) QW till end of C6, optionally |
| | followed by Q2W, dose will be determined based on Example 16, sub- |
| | study B(arm B1, B2 and B3). Elranatamab will be administered |
| | subcutaneously. |
| | Lenalidomide: 10 mg PO. Cycle 1: daily from Day 8 (or Day 15) to Day |
| | 28. Cycle 2 and onwards: From Day 1 to Day28, may escalate to 15 |
| | mg PO daily after 3 cycles |
| B | Lenalidomide: 10 mg PO daily, from Day 1 to Day 28 of each 28-day |
| | cycle; may escalate to 15 mg PO daily after 3 cycles. |

Premedications are required approximately 60 minutes prior to both priming dose (on C1D1 and C1D4) and the first full dose of elranatamab (C1D8). Premedications to be used are, acetaminophen 650 (or paracetamol 500 mg), diphenhydramine 25 mg, oral or IV, and dexamethasone 20 mg (or equivalent) oral or IV.

### Example 15. A Randomized, Controlled, 2-Arm Phase 3 Study of Elranatamab (PF06863135) and Lenalidomide Versus Control in Patients with Newly Diagnosed Multiple Myeloma (NDMM) Who are Ineligible for Stem Cell Transplant

The objectives of this study include to compare the efficacy of elranatamab plus lenalidomide combination therapy (Arm A) with the lenalidomide control arm and to determine the safety and tolerability of elranatamab. Participants in the study will be those with newly diagnosed multiple myeloma who are ineligible to stem-cell transplantation. Table 17 below describes the planned dosing regiments for each arm of the study.

TABLE 17
Combination Therapy of Elranatamab and Lenalidomide for NDMM
Patients who are Stem Cell Transplant Ineligible Study Treatments

| Arms | Drugs and Dosing |
|---|---|
| Experimental | Induction, Elranatamab: TBD based |
| Arm | on results of Example 16, arm |
| | B1, B2 and B3. lenalidomide: 5-25 mg PO daily on 14-21 |
| | consecutive days in each cycle, cycle 1 starts on day 1 week 3; |
| | dexamethasone: 10, 20, 40 mg oral daily on selected days (e.g. |
| | day 1, 8, 15, 22) in at least cycle 1 and cycle 2. |
| | Maintenance, elranatamab: TBD based on results of Example 16, |
| | arm B1, B2 and B3; lenalidomide: 5-15 mg PO QD D1-D28 each |
| | cycle |
| Control Arm | Induction, Lenalidomide, Bortezomib, |
| | Dexamethasone, standard dose and schedule |
| | Maintenance, lenalidomide, standard dose and schedule |

### Example 16. Phase 1b and 2, Open Label, Umbrella Study of Elranatamab (PF06863135), in Combination with Other Anti-Cancer Treatments in Participants with Relapsed/Refractory Multiple Myeloma (RRMM)

The objectives of the study include to assess safety and tolerability of elranatamab in combination with other anti-cancer therapies in participants with RRMM in order to select RP2D for the combination. Table 18 describes a few exemplary combination therapy trial designs of this study.

TABLE 18
Combination Therapy of Elranatamab and other Anti-Cancer Therapy
for Relapsed/Refractory Multiple Myeloma (RRMM) Study Treatments

| Arms | Drugs and Dosing |
|---|---|
| A1 | Elranatamab: priming dosing of 12 mg + 32 mg in week 1 followed by |
| | full dose of 44 mg (or 76 mg) Q1W for 23 or 24 weeks, with the option |
| | of reducing to 44 mg (or 76 mg) Q2W thereafter. |
| | Sasanlimab; 300 mg Q4W |
| A2 | Elranatamab: priming dosing of 12 mg + 32 mg in week 1 followed by |
| | full dose of 44 mg (or 76 mg) Q1W for 23 or 24 weeks, with the option |
| | of reducing to 44 mg (or 76 mg) Q2W thereafter. |
| | Isatuximab: TBD |
| B1 | Elranatamab: priming dosing of 12 mg + 32 mg in week 1 followed by |
| | full dose of 44 mg (or 76 mg) Q1W for 25 weeks, with the option of |
| | reducing to 44 mg (or 76 mg) Q2W thereafter, 76, |
| | Lenalidomide: 5-25 mg PO QD, day 1-14 of 21 day cycle or 1-21 of |
| | 28 day cycle, cycle 1 starts on day 1 of week 3. |
| | Dexamethasone: 10, 20, 40 mg oral daily on selected days (e.g. day 1, |
| | 8, 15, 22) in at least cycle 1 and cycle 2. |
| B2 | TBD results of B1 |
| | Elranatamab: priming dosing of 12 mg + 32 mg in week 1 followed by |
| | full dose of 44 mg (or 76 mg) Q1W for 25 weeks, with the option of |
| | reducing to 44 mg (or 76 mg) Q2W thereafter, 76, |
| | Lenalidomide: 5-25 mg PO QD, day 1-14 of 21 day cycle or 1-21 of |
| | 28 day cycle, cycle 1 starts on day 1 of week 3. |
| | Dexamethasone: 10, 20, 40 mg oral daily on selected days (e.g. day 1, |
| | 8, 15, 22) in at least cycle 1 and cycle 2. |
| B3 | Repeat the dosing of arm B1 or arm B2 but in dose expansion |

### Sequences

Table 19 lists the sequences of the BCMA×CD3 bispecific antibody PF-06863135 and the PD-1 antibody sasanlimab, and the corresponding SEQ ID NOs as referred to herein. SEQ ID NOs 1 to 13 are the sequences of the CD3 arm of PF-06863135, SEQ ID NOs 14 to 26 are the sequences of the BCMA arm of PF-06863135. SEQ ID NOs 27 to 34 are the sequences of the PD-1 antibody sasanlimab.

TABLE 19
Sequences of PF-06863135 and sasanlimab

| SEQ ID NO | |
|---|---|
| (descriptions) | Sequences |
| 1 | EVQLVESGGG LVQPGGSLRL SCAASGFTFS |
| (CD3 arm VH) | DYYMTWVRQA PGKGLEWVAF IRNRARGYTS |
| | DHNPSVKGRF TISRDNAKNS LYLQMNSLRA EDTAVYYCAR |
| | DRPSYYVLDY WGQGTTVTVSS |
| 2 | DYYMT |
| (CD3 arm VH | |
| CDR1) | |
| 3 | GFTFSDY |
| (CDR3 VH | |
| CDR1) | |
| 4 | GFTFSDYYMT |
| (CD3 arm VH | |
| CDR1) | |
| 5 | RNRARGYT |
| (CD3 arm VH | |
| CDR2) | |
| 6 | FIRNRARGYTSDHNPSVKG |
| (CD3 arm VH | |
| CDR2) | |
| 7 | DRPSYYVLDY |
| (CD3 arm VH | |
| CDR3) | |
| 8 | EVQLVESGGG LVQPGGSLRL SCAASGFTFS |
| (CD3 arm full | DYYMTWVRQA PGKGLEWVAF IRNRARGYTS |
| heavy chain) | DHNPSVKGRF TISRDNAKNS LYLQMNSLRA EDTAVYYCAR |
| | DRPSYYVLDY WGQGTTVTVS Sastkgpsvf plapcsrsts |
| | estaalgclv kdyfpepvtv swnsgaltsg vhtfpavlqs sglyslssvv |
| | tvpssnfgtq tytcnvdhkp sntkvdktve rkervreprc pappvagpsv |
| | flfppkpkdt lmisrtpevt cvvvavshed pevqfnwyvd gvevhnaktk |
| | preeqfnstf rvvsvltvvh qdwlngkeyk ckvsnkglps siektisktk |
| | gqprepqvyt lppsreemtk nqvsltclvk gfypsdiave wesngqpenn |
| | ykttppmlds dgsfflysrl tvdksrwqqg nvfscsvmhe alhnhytqks lslspgk |
| 9 | DIVMTQSPDS LAVSLGERAT INCKSSQSLF NVRSRKNYLA |
| (CD3 arm VL) | WYQQKPGQPP KLLISWASTR ESGVPDRFSG SGSGTDFTLT |
| | ISSLQAEDVA VYYCKQSYDL FTFGSGTKLE IK |
| 10 | KSSQSLFNVRSRKNYLA |
| (CD3 arm VL | |
| CDR1) | |
| 11 | WASTRES |
| (CD3 arm VL | |
| CDR2) | |
| 12 | KQSYDLFT |
| (CD3 arm VL | |
| CDR3) | |
| 13 | DIVMTQSPDS LAVSLGERAT INCKSSQSLF NVRSRKNYLA |
| (CD3 arm full | WYQQKPGQPP KLLISWASTR ESGVPDRFSG SGSGTDFTLT |
| light chain) | ISSLQAEDVA VYYCKQSYDL FTFGSGTKLE IKrtvaapsv |
| | fifppsdeql ksgtasvvcl lnnfypreak vqwkvdnalq sgnsqesvte |
| | qdskdstysl sstltlskad yekhkvyace vthqglsspv tksfnrgec |
| 14 | EVQLLESGGG LVQPGGSLRL SCAASGFTFS SYPMSWWRQA |
| (BCMA arm VH) | PGKGLEWVSA IGGSGGSLPY ADIVKGRFTI SRDNSKNTLY |
| | LQMNSLRAED TAVYYCARYW PMDIWGQGTL VTVSS |
| 15 | GFTFSSY |
| (BCMA arm VH | |
| CDR1) | |
| 16 | SYPMS |
| (BCMA arm VH | |
| CDR1) | |
| 17 | GFTFSSYPMS |
| (BCMA arm VH | |
| CDR1) | |
| 18 | GGSGGS |
| (BCMA arm VH | |
| CDR2) | |
| 19 | AIGGSGGSLPYADIVKG |
| (BCMA arm VH | |
| CDR2) | |
| 20 | YWPMDI |
| (BCMA arm VH | |
| CDR3) | |
| 21 | EVQLLESGGG LVQPGGSLRL SCAASGFTFS SYPMSWWRQA |
| (BCMA arm full | PGKGLEWVSA IGGSGGSLPY ADIVKGRFTI SRDNSKNTLY |
| heavy chain) | LQMNSLRAED TAVYYCARYW PMDIWGQGTL VTVSSastkg |
| | psvfplapcs rstsestaal gcivkdyfpe pvtvswnsga Itsgvhtfpa |
| | vlqssglysl ssvvtvpssn fgtqtytcnv dhkpsntkvd ktverkceve |
| | cpecpappva gpsvflfppk pkdtlmisrt pevtcvvvav shedpevqfn |
| | wyvdgvevhn aktkpreeqf nstfrvvsvl tvvhqdwlng keykckvsnk |
| | glpssiekti sktkgqprep qvytlppsre emtknqvslt cevkgfypsd |
| | iavewesngq pennykttpp mldsdgsffl yskltvdksr wqqgnvfscs |
| | vmhealhnhy tqkslslspg k |
| 22 | EIVLTQSPGT LSLSPGERAT LSCRASQSVS SSYLAWYQQK |
| (BCMA arm VL) | PGQAPRLLMY DASIRATGIP DRFSGSGSGT DFTLTISRLE |
| | PEDFAVYYCQ QYQSWPLTFG QGTKVEIK |
| 23 | RASQSVSSSYLA |
| (BCMA arm VL | |
| CDR1) | |
| 24 | DASIRAT |
| (BCMA arm VL | |
| CDR2) | |
| 25 | QQYQSWPLT |
| (BCMA arm VL | |
| CDR3) | |
| 26 | EIVLTQSPGT LSLSPGERAT LSCRASQSVS SSYLAWYQQK |
| (BCMA arm full | PGQAPRLLMY DASIRATGIP DRFSGSGSGT DFTLTISRLE |
| light chain) | PEDFAVYYCQ QYQSWPLTFG QGTKVEIKrt vaapsvfifp |
| | psdeqlksgt asvvcllnnf ypreakvqwk vdnalqsgns qesvteqdsk |
| | dstyslsstl tlskadyekh kvyacevthq glsspvtksf nrgec |
| 27 | QVQLVQSGAE VKKPGASVKV SCKASGYTFT SYWINWWRQA |
| (PD-1 VH) | PGQGLEWMGN IYPGSSLTNY NEKFKNRVTM TRDTSTSTVY |
| | MELSSLRSED TAVYYCARLS TGTFAYWGQG TLVTVSS |
| 28 | SYWIN |
| (PD-1 VH | |
| CDR1) | |
| 29 | NIYPGSSLTNYNEKFKN |
| (PD-1 VH | |
| CDR2) | |
| 30 | LSTGTFAY |
| (PD-1 VH | |
| CDR3) | |
| 31 | DIVMTQSPDS LAVSLGERAT INCKSSQSLW DSGNQKNFLT |
| (PD-1 VL) | WYQQKPGQPP KLLIYWTSYR ESGVPDRFSG SGSGTDFTLT |
| | ISSLQAEDVA VYYCQNDYFY PHTFGGGTKV EIK |
| 32 | KSSQSLWDSGNQKNFLT |
| (PD-1 VL | |
| CDR1) | |
| 33 | WTSYRES |
| (PD-1 VL | |
| CDR2) | |
| 34 | QNDYFYPHT |
| (PD-1 VL | |
| CDR3) | |

It is claimed:

1.-51. (canceled)

52. A method of treating cancer, comprising administering to the subject a combination therapy which comprises a first therapeutic agent and a second therapeutic agent, wherein the first therapeutic agent is a BCMA bispecific antibody and the second therapeutic agent is an anti-PD-1 antibody or an anti-PD-L1 antibody.

53. The method of claim 52, wherein the BCMA bispecific antibody is PF-06863135, and the second therapeutic agent is an anti-PD-1 antibody.

54. The method of claim 53, wherein the anti-PD-1 antibody is sasanlimab.

55. The method of claim 52, wherein the BCMA bispecific antibody is PF-06863135, the second therapeutic agent is an anti-PD-1 antibody sasanlimab and the cancer is multiple myeloma.

56. A method of treating cancer, comprising administering to the subject a combination therapy which comprises a first therapeutic agent and a second therapeutic agent, wherein the first therapeutic agent is BCMA bispecific antibody and the second therapeutic agent is a gamma secretase inhibitor.

57. The method of claim 56, wherein the BCMA bispecific antibody is PF-06863135.

58. The method of claim 56, wherein the BCMA bispecific antibody is PF-06863135, and the gamma secretase inhibitor is nirogacestat, or a pharmaceutically acceptable salt thereof.

59. The method of claim 56, wherein the BCMA bispecific antibody is PF-06863135, the gamma secretase inhibitor is nirogacestat, or a pharmaceutically acceptable salt thereof, and the cancer is multiple myeloma.
